(19) United States
(12) Patent Application Publication
(10) Pub. No.: US 2002/0151526 A1
(43) Pub. Date: Oct. 17, 2002

(54) Bile-acid prodrugs of L-dopa and their use in the sustained treatment of parkinsonism

(76) Inventors: Mark A. Gallop, Los Altos, CA (US); Kenneth C. Cundy, Redwood City, CA (US); Cindy X. Zhou, Palo Alto, CA (US)

Correspondence Address: BURNS DOANE SWECKER & MATHIS L L P, POST OFFICE BOX 1404, ALEXANDRIA, VA, 22313-1404, US

(21) Appl. No.: 09/972,431

(22) Filed: Oct. 5, 2001

Related U.S. Application Data
(60) Provisional application No. 60/238,758, filed on Oct. 6, 2000.
(60) Provisional application No. 60/297,654, filed on Jun. 11, 2001.

(51) Int. Cl. A61K 31/66; A61K 31/57; C07J 5/00; C07J 7/00; C07J 1/00

(52) U.S. Cl. 514/143; 514/178; 552/506; 552/557

(57) ABSTRACT

Bile-acid conjugates useful for sustained release of L-DOPA, inhibitors of catechol O-methyl transferase and/or inhibitors of L-aromatic amino acid decarboxylase are provided.

### CROSS-REFERENCE TO RELATED APPLICATIONS

This application claims the benefit of U.S. Provisional Application Serial No. 60/238,758, which was filed on Oct. 6, 2000; and U.S. Provisional Application Serial No. 60/297,654, which was filed on Jun. 11, 2001, the disclosures of which are incorporated by reference in their entirety

### BACKGROUND OF THE INVENTION

1. Field of the Invention

This invention is directed to compounds and pharmaceutical compositions for sustained release, when orally delivered to a mammalian patient, of levodopa (L-DOPA or L-dihydroxyphenylalanine), L-aromatic amino acid decarboxylase (AADC) inhibitors and/or catechol O-methyl transferase (COMT) inhibitors. These compounds and pharmaceutical compositions are useful in treating Parkinson's disease in such patients.

This invention is also directed to methods for treating Parkinson's disease in a mammalian patient by administering the compounds or pharmaceutical compositions described herein to the patient. One advantage of the compounds, compositions and methods of this invention is their ability to maintain a sustained release of drug in the mammalian patient.

### References

The following publications, patents and patent applications are cited in this application as superscript numbers:

¹Cheng, et al., Xenobiotica, 1976, 6:237-248.

²Contin, et al., Clin. Pharmakinet., 1996, 30:463-481.

³Cooper, et al., J. Pharm. Pharmacol. 1987, 39:809.

⁴Genta Jago product licensing information, June 1997.

⁵Greene et al., Protective Groups in Organic Synthesis, Third Edition, John Wiley & Sons, 1999, and references cited therein.

⁶Fix, et al., Pharm. Res., 1989, 6:501-5.

⁷Fix, et al., Pharm. Res., 1990, 4:384-7.

⁸Iwamoto, et al., J. Pharm. Pharmacol., 1987, 39:421-5.

⁹Juncos, et al., Neurology 1987, 37:1742.

¹⁰Kurlan, et al., Ann. Neurol., 1988, 23:589-59.

¹¹Larock, Comprehensive Organic Transformations, John Wiley & Sons, Second Edition, 1999.

¹²Leppert, et al., Pharm. Res., 1988, 5:587-591.

¹³March, Advanced Organic Chemistry, John Wiley & Sons, Fourth Edition, 1992.

¹⁴Physician's Desk Reference

¹⁵Sandler, et al., Lancet, 1974, 16:238-240.

¹⁶Smith, Organic Synthesis, John Wiley & Sons, 1994.

¹⁷Yeh, et al., Neurology, 1989, 39:25-38.

¹⁸ “Novel dopa/dopamine prodrugs”, U.S. Pat. No. 4,311,706, Jan. 19, 1982.

¹⁹ “Rectally absorbable form of L-dopa”,U.S. Pat. No. 4,663, 349, May 5, 1987

²⁰ “Rectally absorbable form of L-dopa”,U.S. Pat. No. 4,771,073, Sept. 13, 1988

²¹ “Pharmaceutical compositions containing levodopa methyl ester, preparation and therapeutic applications thereof”, U.S. Pat. No. 4,826,875, May 2, 1989

²² “Rectally absorbable form of L-dopa”,U.S. Pat. No. 4,873,263, Oct. 10, 1989.

²³ “L-dopa derivatives of their acid addition salts, process for producing same and their use”, U.S. Pat. No. 4,966,915, Oct. 30, 1990.

²⁴ “Method to treat Parkinsons disease”, U.S. Pat. No. 5,017,607, May 21, 1991.

²⁵ “Modified bile acid conjugates, and their use as pharmaceuticals”, U.S. Pat. No. 5,462,933, Oct. 31, 1995.

²⁶ “L-dopa ethyl ester to treat Parkinson's disease”, U.S. Pat. No. 5,607,969, Mar. 4, 1997.

²⁷ “Pharmaceutical composition of L-DOPA ester”,U.S. Pat. No. 5,840,756, Nov. 24, 1998.

²⁸ “Means to achieve sustained release of synergistic drugs by conjugation”, U.S. Pat. No. 6,051,576, Apr. 18, 2000.

²⁹ “Codrugs as a method of controlled drug delivery”, WO95/20567, Aug. 8, 1995.

³⁰U.S. Provisional Application No. 60\023,758 (Attorney Docket Number 033053-005) filed on Oct. 6, 2000.

³¹“L-Dopa Derivatives or Their Acid AdditionSalts, Process for Producing Same and Their Use”,EP 0 309 827, Apr. 15, 1989.

All of the above publications, patents and patent applications are herein incorporated by reference in their entirety to the same extent as if each individual publication, patent or patent application was specifically and individually indicated to be incorporated by reference in its entirety.

2. State of the Art

Levodopa (L-DOPA) is a prodrug of dopamine that is considered as the first line of treatment for Parkinsonism in mammalian patients and, in particular, human patients. Following oral administration, levodopa is rapidly absorbed via the large neutral amino acid transporter present in the upper small intestine. Due to the narrow distribution of this transport system, the window for opportunity for levodopa absorption is limited and the extent of absorption is dependent on the rate of gastric emptying of the drug. Once it has passed the small intestine, levodopa is poorly absorbed from the large intestine¹². Only about 30-50% of the administered dose reaches the systemic circulation after oral administration. The absolute bioavailability of levodopa is dose-dependent, due to saturation of the active transport pathway.¹ Plasma levels of levodopa must be carefully titrated for each patient to achieve the optimal therapeutic activity. If the concentration of levodopa is too low in plasma (and consequently in the brain), the patient may experience a return of the symptoms of Parkinson's disease (rigidity, tremor, bradykinesia, etc.). If plasma drug levels are too high, toxic side effects may occur. Uncontrolled fluctuations in plasma levodopa levels may greatly contribute to the incidence of “on-off” fluctuations (dyskinesias). The most effective control of Parkinsonism is observed when plasma levels of levodopa are maintained in a narrow range, for example, by continuous intraduodenal infusion¹⁰.

Following absorption, levodopa is rapidly converted to dopamine by L-aromatic amino acid decarboxylase (AADC) in the intestines and the liver. It has been shown that intestinal metabolism of levodopa is the major source of first pass loss of the drug. Intraportal and intravenous administration gave similar levodopa systemic exposures in rats.⁸ In patients, less than 1% of the administered dose reaches the CNS intact, following transport across the blood-brain barrier by the neutral amino acid transporter. For this reason, levodopa is normally coadministered with a drug designed to inhibit its peripheral decarboxylation (e.g., carbidopa or benserazide). When administered with carbidopa, intact levodopa is transported into the CNS where it can be converted to dopamine. Carbidopa itself does not cross the blood-brain barrier and, therefore, does not inhibit the required conversion of levodopa to dopamine in the brain.

The oral bioavailability of levodopa from conventional formulations of levodopa/carbidopa (e.g. Sinemet) is 84-99%.^{14,17} The half-life of levodopa in the plasma of patients is about 50 minutes when administered alone, or 1 to 2 hours when coadministered with carbidopa. For this reason, the drug must be administered three or more times per day. There is clearly a need for a formulation that would deliver a sustained level of L-dopa to the systemic circulation allowing once or twice per day dosing. Such a formulation would be more convenient for patients while reducing the incidence of “on-off” oscillations resulting from fluctuations in plasma levels of drug.

A formulation of levodopa/carbidopa (Sinemet CR) intended to provide a controlled release of both drugs is commercially available. Sinemet CR is designed to release both levodopa and carbidopa over a 4 to 6 hour period. However, absorption of levodopa is limited to the small intestine and the resulting bioavailability of levodopa from Sinemet CR is reduced relative to the immediate release product. In most cases, Sinemet CR must also be given more than twice per day to achieve a therapeutic level of levodopa. Conventional formulation approaches that target the large intestine are ineffective for the sustained delivery of levodopa. A simple enteric coated formulation of levodopa led to increased gastrointestinal side effects (nausea) but did not improve absorption.¹⁵ A sustained release formulation of levodopa/carbidopa has been described that employs a swellable matrix (Geomatrix) delivery system to retain the drug in the stomach.⁴ However, this formulation was designed to be bioequivalent to the commercially available Sinemet CR formulation and therefore has not proven capable of providing the desired goal of a once or twice per day regimen.

In addition to decarboxylation by AADC, substantial amounts of levodopa are metabolized by the enzyme catechol O-methyl transferase (COMT), with the greatest activity localized in the liver and kidneys. The resulting product, 3-O-methyl dopa (3-OMD), has a plasma half-life of 15 hours and accumulates during long-term levodopa therapy. 3-OMD, like levodopa, is a substrate for the large neutral amino acid transport system in the brain and can competitively inhibit uptake of levodopa from plasma to brain.² The nitrocatechol compounds entacapone, nitecapone and tolcapone are selective COMT inhibitors that are used clinically to block the peripheral O-methylation of levodopa. These compounds produce a significant (up to 50%) increase in half-life and the area-under-the curve (AUC) of levodopa when used as an adjunct to levodopa-carbidopa regimens.

The potential use of various simple esters as prodrugs of levodopa as a means to improve the pharmacokinetics of the drug has been proposed.^{3,9,18-22,23} An oral formulation of levodopa methyl ester (Levomet, CHF 1301) has been described (Chiesi Pharmaceuticals). The ethyl ester of levodopa (TV-1203) is under clinical investigation as a potential therapy for Parkinsonism when coadministered with carbidopa.²⁶ A sustained release formulation of levodopa ethyl ester in a mixture of hydroxypropylmethyl cellulose, hydroxypropyl cellulose, and a carboxyvinyl polymer has been described.²⁷ However, oral administration of this formulation to healthy adults pretreated with carbidopa produced a plasma levodopa terminal half-life of only 2 hours, comparable to that of Sinemet CR. This result indicates that the ester was absorbed faster than the rate of its hydrolysis to levodopa. A pivaloyl ester of levodopa (NB-355) has been described.²¹ Conversion of the prodrug to levodopa in rat plasma following absorption from an intestinal loop was slow and sustained levels of prodrug were observed, while levels of levodopa were low. The potential for using ester prodrugs of levodopa to enhance rectal absorption of the drug has been described.^{19,20,22} Notably, the absorption of simple alkyl esters of levodopa has been shown to be greater following rectal absorption than following oral dosing.^{6,7} This effect is due to the decreased abundance of esterases in the large intestine relative to the small intestine. Therefore, selective delivery of a prodrug of levodopa to the large intestine in a sustained release formulation would be expected to provide a greater oral biovailability and a prolonged exposure to the drug.

The half-life of levodopa is prolonged and its bioavailability increased by the coadministration of carbidopa. Both drugs have relatively short half-lives (˜2 hours).¹⁷ Any method of sustained delivery of levodopa to the systemic circulation would, therefore, require a sufficient level of carbidopa to continuously inhibit peripheral decarboxylation of levodopa. In order to avoid the need for frequent (more than twice per day) dosing of carbidopa, it is necessary to deliver both levodopa and carbidopa (or prodrugs thereof) in a sustained manner. It has been proposed that rectal coadministration of an AADC inhibitor such as carbidopa with an ester prodrug of levodopa would be possible as a means to decrease metabolic clearance of levodopa.^{19,20,22} However, studies in rats have since indicated that absorption of carbidopa following rectal administration is poor.¹² Carbidopa therefore appears to be preferentially absorbed in the small intestine, presumably by an active transport mechanism. For this reason, a conventional sustained release formulation of carbidopa is unlikely to achieve the desired result of sustained systemic exposure. Therefore, any combination of carbidopa with either levodopa or a prodrug of levodopa in a sustained release formulation will fail to provide the required protection from peripheral decarboxylation and will not achieve the necessary sustained level of levodopa in the brain.

### SUMMARY OF THE INVENTION

This invention is directed, in part, to novel prodrugs of levodopa, the AADC inhibitors, e.g., carbidopa and benserazide, and/or the COMT inhibitors, e.g., entacapone, nitecapone and tolcapone, each of which is capable of undergoing absorption across the intestinal epithelium and enterohepatic recirculation via active transport through the bile acid transport system. Upon oral administration, these prodrugs are cleaved within the enterohepatic system to release the parent drug and/or an active metabolite thereof into the systemic circulation. Significantly, only a fraction (typically<50%) <50%) of the prodrug is cleaved during each pass through the enterohepatic cycle. Thus, the enterohepatic circulation serves as a reservoir of the drug enabling sustained systemic drug levels to be achieved. One aspect of the present invention is related to prodrugs of levodopa, the AACD inhibitor and/or COMT inhibitor that can provide sustained release of levodopa, the AACD inhibitor and/or COMT inhibitor in a mammalian patient after oral administration of the prodrug.

For anti-Parkinson therapy, it may be advantageous to coadminister to patients recirculating prodrugs of levodopa together with similar prodrugs of AACD and/or COMT inhibitors. In this manner, one can sustain the level of levodopa in the peripheral circulation ensuring that therapeutic drug levels can be sustained within the brain. Another aspect of the present invention is related to prodrugs of levodopa and the AACD inhibitor or prodrugs of levodopa and the COMT inhibitor that can provide sustained release of levodopa and the AACD inhibitor or COMT inhibitor in a mammalian patient after oral administration of the prodrug.

Preferred prodrugs of this invention are bile acid conjugates of the aforementioned drugs. Naturally occurring bile acids such as cholic acid, chenodeoxycholic acid, ursodeoxycholic acid, deoxycholic acid, ursocholic acid and lithocholic acid are particularly preferred. The site of conjugation of these bile acids to the drugs is preferably via the 3-hydroxy group or the C-24 carboxyl moiety, as illustrated in FIG. 1. Optionally, a cleavable linker functionality (Y or Y′ in formula (I) below) may be introduced between the drug and the bile acid and this linker may be selected such that its rate of cleavage in vivo is optimized to produce the desired degree of sustained systemic exposure to the drug.

In one embodiment this invention is directed to prodrugs of the formula D-Y-T, wherein D represents bile acid conjugates of the aforementioned drugs and Y is a cleavable linker, and T is a substrate for an intestineal bile acid transporter.

The prodrugs of the present invention are preferably compounds represented by formula (I):

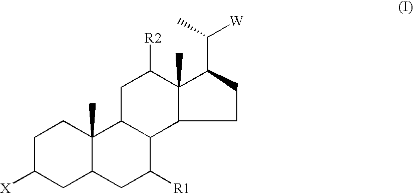

wherein R¹ is selected from the group consisting of hydrogen and OH;

R² is selected from the group consisting of hydrogen and OH;

X is selected from the group consisting of OH and D-Y-, where Y is selected from the group consisting of a covalent bond and a cleavable linker group covalently connecting D to the steroid;

D is a member selected from the group consisting of L-DOPA, a catechol O-methyl transferase inhibitor, an inhibitor of a L-aromatic amino acid decarboxylase, and derivatives of L-DOPA;

W is selected from the group consisting of (a) a substituted alkyl group containing a moiety which is negatively charged at physiological pH, which moiety is selected from the group consisting of —COOH, —SO₃H, —SO₂H, —P(O)(OR⁶)(OH), —OP(O)(OR⁶)(OH), —OSO₃H and the like and pharmaceutically acceptable salts thereof, where R⁶ is selected from the group consisting of alkyl, substituted alkyl, aryl and substituted aryl; and (b) a group of the formula:

-M-Y′-D′

where:

M is selected from the group consisting of —CH₂OC(O)— and —CH₂CH₂C(O)—;

Y′ is a covalent bond or a cleavable linker group covalently connecting D′ to M;

D′ is a member selected from the group consisting of L-DOPA, a catechol O-methyl transferase inhibitor, an inhibitor of a L-aromatic amino acid decarboxylase, and derivatives of L-DOPA;

with the proviso that either X is -Y-D and/or W is -M-Y′-D′

wherein the compound of formula (I) above is a substrate for an intestinal bile acid transporter;

or a pharmaceutically acceptable salt thereof.

The linker groups Y and Y′ are more preferably represented by the formula -X^{*}-Y^{*}-Z- where X^{*} is the linker chemistry for attachment to the drug D or D′; Y^{*} is a covalent bond or a linker moiety; and Z is the linker chemistry for attachment to the steroid.

Preferably X^{*} is selected from the group consisting of —OC(O)—, —OC(O)NR⁷—, —OC(O)OCR¹¹R¹²O—, —OC(O)OCR¹¹R¹²OC(O)—, —OC(O)OCR¹¹R¹²OC(O)O—, —OC(O)OCR¹¹R¹²OC(O)NR⁷—, —NR⁷C(O)O—, —NR⁷C(O)—, —NR⁷C(O)OCR¹¹R¹²OC(O)—, —NR⁷C(O)OCR¹¹R¹²OC(O)O—, —NR⁷CH₂NR⁷C(O)—, —C(O)O—, —C(O)S—, —C(O)NR⁷—, —C(O)NR⁷C(O)R⁷—, —C(O)OCR¹¹R¹²O—, —C(O)OCR¹¹R¹²OC(O)—, —C(O)OCR¹¹R¹²OC(O)O—, —C(O)OCH₂C(O)NR⁷—, —C(O)OCH₂CH₂NR⁷C(O)—, —C(O)OCH₂NR⁷C(O)—, —C(O)OCR¹¹R¹²OC(O)NR⁷—, with the underlined atom being derived from a hydroxyl, NH, or carboxylic acid moiety of the drug D or D′;

each R⁷ is independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl; R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring.

Preferably Z is selected from the group consisting of a bond, —O—, —S—, —C(O)O—, —OC(O)O—, —NR⁷C(O)O—, —OC(O)NR⁷—, —OP(O)(OR⁶)O—, —P(O)(OR⁶)O—, —NR⁷P(O)(OR⁶)O—, —C(O)NR⁷—, —NR⁷C(O)NR⁷—, —NR⁷C(O)NR⁷—, —S(O)₂NR⁷—, —S(O)—, —S(O)₂—, —C(O)S—, —ON═, —C(O)ON═, —NR⁷C(O)ON═, —C(O)OCR¹¹R¹²ON═, and a C═C linkage, wherein R⁶—R¹² are defined as above.

Preferably Y^{*} is a bond or a bivalent hydrocarbyl radical of 1 to 18 atoms having at least one alkylene, alkenylene or alkynylene group, with at least one alkylene, alkenylene or alkynylene group optionally replaced with —O—, —S—, —NR⁷—, —C(O)—, —C(S)—, —OC(O)—, —C(O)O—, —SC(O)—, —C(O)S—, —SC(S)—, —C(S)S—, —C(O)NR⁷—, —NR⁷C(O)—, arylene, substituted arylene, cycloalkylene, substituted cycloalkylene, cycloalkenylene, substituted cycloalkenylene, bivalent heterocyclic group or substituted bivalent heterocyclic group.

Y^{*} is also preferably represented by the formula:

—(R^{3′})_{f}(R^{4′})_{g}(R^{5′})ₕ—

where each of R^{3′}, R^{4′} and R^{5′} are independently selected from the group consisting of alkylene, substituted alkylene, alkenylene, substituted alkenylene, alkynylene, substituted alkynylene, cycloalkylene, substituted cycloalkylene, cycloalkenylene, substituted cycloalkenylene, arylene, substituted arylene, heteroarylene, substituted heteroarylene, heterocyclene and substituted heterocyclene; and each of f, g and h are independently an integer from 0 to 3. More preferably, Y^{*} is alkylene, alkenylene or alkynylene.

One preferred group of prodrugs of the present invention are compounds represented by formula (I-a):

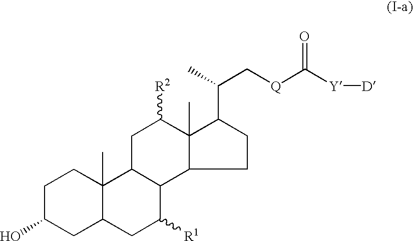

wherein:

Y′ is selected from the group consisting of a covalent bond and a cleavable linker group covalently connecting D′ to the C-24 position of the steroid;

D′ is a member selected from the group consisting of L-DOPA, a catechol O-methyl transferase inhibitor, an inhibitor of a L-aromatic amino acid decarboxylase, and derivatives of L-DOPA;

Q is CH₂ or O;

R¹ is selected from the group consisting of H and OH;

R² is selected from the group consisting of H and OH;

wherein the compound of formula (I-a) above is a substrate for an intestinal bile acid transporter; or

pharmaceutically acceptable salts thereof.

Particularly preferred prodrugs of formula (I-a) are compounds represented by formulae (I-a-1) and (I-a-2):

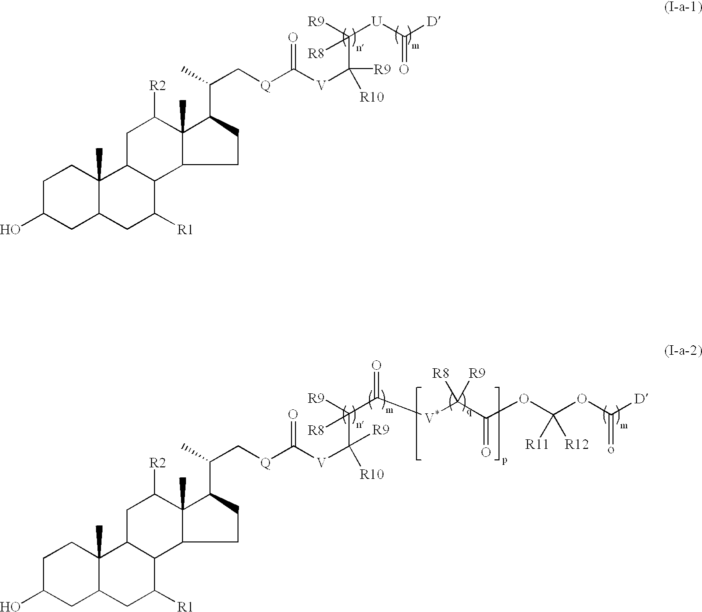

wherein:

D′ is a member selected from the group consisting of L-DOPA, a catechol O-methyl transferase inhibitor, an inhibitor of a L-aromatic amino acid decarboxylase, and derivatives of L-DOPA;

Q is CH₂ or O;

R¹ is selected from the group consisting of H and OH;

R² is selected from the group consisting of H and OH;

V and V^{*} are independently NR⁷, O, S or CR⁸R⁹;

U is NR⁷, O, S;

R¹⁰ is R⁸ or (CR⁸R⁹)ᵣT;

T is selected from the group consisting of CO₂H, SO₃H, OSO₃H, SO₂H, P(O)(OR⁶)(OH), OP(O)(OR⁶)(OH) and pharmaceutically acceptable salts thereof;

each m is 0 or 1;

n′ is 0, 1, 2, 3 or 4;

p is 0, 1,2 ,3 ,4, 5, or 6;

each q is independently 1, 2, 3, 4, 5, or 6;

r is 0 or 1;

R⁶ is selected from the group consisting of alkyl, substituted alkyl, aryl and substituted aryl;

R⁷, R⁸ and R⁹ are independently hydrogen, alky, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or, when R⁷ and R⁹ are present and attached to adjacent atoms, then R⁷ and R⁹ together with the atoms to which they are attached form a cycloalkyl, substituted cycloalkyl, heterocycle or substituted heterocyclic ring;

R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring;

wherein the compound of formulae (I-a-1) and (I-a-2) above is a substrate for an intestinal bile acid transporter; or

pharmaceutically acceptable salts thereof.

Another preferred group of prodrugs of the present invention are compounds represented by formula (I-b):

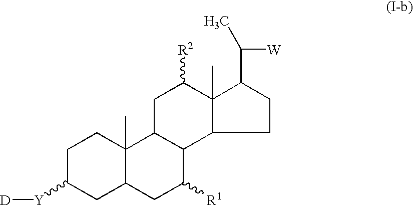

wherein:

Y is selected from the group consisting of a covalent bond and a cleavable linker group covalently connecting D to the steroid;

D is a member selected from the group consisting of L-DOPA, a catechol O-methyl transferase inhibitor, an inhibitor of a L-aromatic amino acid decarboxylase, and derivatives of L-DOPA;

R¹ is selected from the group consisting of H and OH;

R² is selected from the group consisting of H and OH;

W is a substituted alkyl group containing a moiety which is negatively charged at physiological pH, which moiety is selected from the group consisting of —COOH, —SO₃H, —SO₂H, —P(O)(OR⁶)(OH), —OP(O)(OR⁶)(OH), —OSO₃H and the like and pharmaceutically acceptable salts thereof, where R⁶ is selected from the group consisting of alkyl, substituted alkyl, aryl and substituted aryl;

wherein the compound of formula (I-b) above is a substrate for an intestinal bile acid transporter;

or pharmaceutically acceptable salts thereof.

Particularly preferred examples of suitable cleavable linkers Y for use in formula (I-b) include structures of formulae (i) through (v) as shown below;

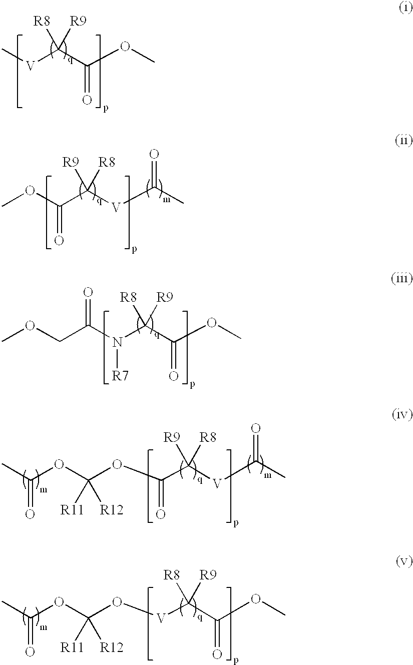

wherein

V is selected from the group consisting of NR⁷, O, S and CR⁸R⁹;

each m is independently 0 or 1;

p is 0, 1,2 ,3 ,4, 5, or 6;

each q is independently 1, 2, 3, 4, 5 or 6;

each R⁷, R⁸ and R⁹ is independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or, when R⁷ and R⁹ are present and attached to adjacent atoms, then R⁷ and R⁹ together with the atoms to which they are attached form a cycloalkyl, substituted cycloalkyl, heterocycle or substituted heterocyclic ring;

R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring.

Still another preferred group of prodrugs of the present invention are compounds represented by formula (I-c):

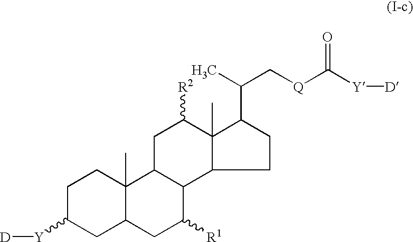

wherein:

Y′ is selected from the group consisting of a covalent bond and a cleavable linker group covalently connecting D′ to the C-24 position of the steroid;

D′ is a member selected from the group consisting of L-DOPA, a catechol O-methyl transferase inhibitor, an inhibitor of a L-aromatic amino acid decarboxylase, and derivatives of L-DOPA;

Y is selected from the group consisting of a covalent bond and a cleavable linker group covalently connecting D to the steroid;

D is a member selected from the group consisting of L-DOPA, a catechol O-methyl transferase inhibitor, an inhibitor of a L-aromatic amino acid decarboxylase, and derivatives of L-DOPA;

Q is CH₂ or O;

R¹ is selected from the group consisting of H and OH;

R² is selected from the group consisting of H and OH;

wherein the compound of formula (I-c) above is a substrate for an

intestinal bile acid transporter;

and pharmaceutically acceptable salts thereof.

Particularly preferred prodrugs of formula (I-c) are compounds represented by formulae (I-c-1) and (I-c-2):

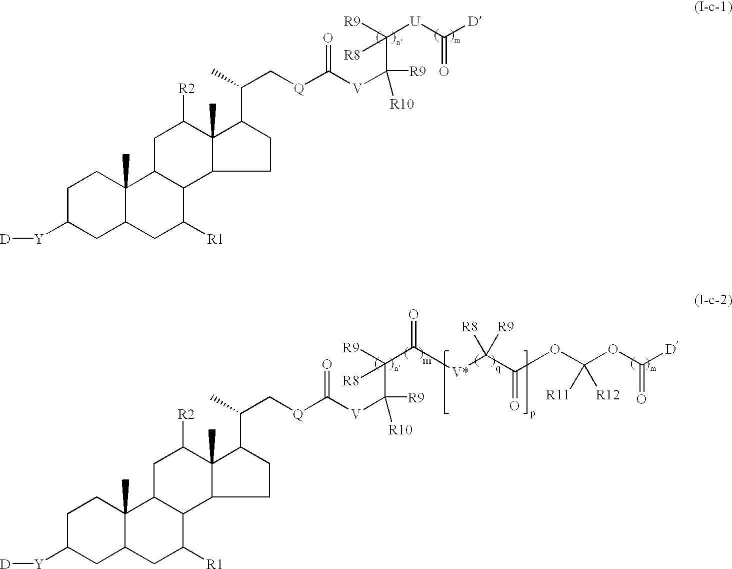

wherein:

D′ is a member selected from the group consisting of L-DOPA, a catechol O-methyl transferase inhibitor, an inhibitor of a L-aromatic amino acid decarboxylase, and derivatives of L-DOPA;

D is a member selected from the group consisting of L-DOPA, a catechol O-methyl transferase inhibitor, an inhibitor of a L-aromatic amino acid decarboxylase, and derivatives of L-DOPA;

Q is CH₂ or O;

R¹ is selected from the group consisting of H and OH;

R² is selected from the group consisting of H and OH;

Y is selected from the group consisting of structures of formulae (i) through (v) below:

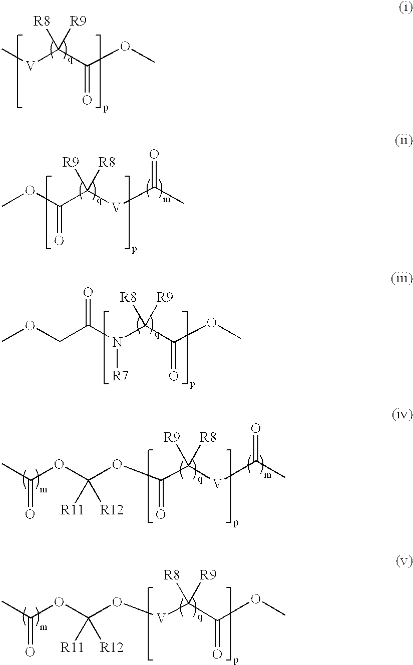

wherein

each V and V^{*} are independently NR⁷, O, S or CR⁸R⁹;

U is NR⁷, O, S;

R¹⁰ is R⁸ or (CR⁸R⁹)ᵣT;

T is selected from the group consisting of CO₂H, SO₃H, OSO₃H, SO₂H, P(O)(OR⁶)(OH), OP(O)(OR⁶)(OH) and pharmaceutically acceptable salts thereof;

each m is 0 or 1;

n′ is 0, 1, 2, 3 or4;

p is 0, 1, 2, 3, 4, 5, or 6;

each q is independently 1, 2, 3, 4, 5, or 6;

r is 0 or 1;

R⁶ is selected from the group consisting of alkyl, substituted alkyl, aryl and substituted aryl;

R⁷, R⁸ and R⁹ are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or, when R⁷ and R⁹ are present and attached to adjacent atoms, then R⁷ and R⁹ together with the atoms to which they are attached form a cycloalkyl, substituted cycloalkyl, heterocycle or substituted heterocyclic ring;

R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring;

wherein the compound of formulae (I-c-1) and (I-c-2) above is a substrate for an intestinal bile acid transporter;

or pharmaceutically acceptable salts thereof.

The compounds described above are preferably administered as pharmaceutical compositions comprising the drug/transporter compound and a pharmaceutically acceptable excipient.

This invention is also directed to methods for treating Parkinson disease in a mammalian patient. One advantage of the compounds, compositions and methods of this invention is their ability to maintain a sustained release of drug in the mammalian patient.

### BRIEF DESCRIPTION OF THE DRAWINGS

FIG. 1 illustrate some preferred embodiments of the bile acid prodrugs for sustained release of L-DOPA and inhibitors of L-DOPA metabolism. In addition, these figures illustrate the formulae of L-DOPA, preferred AADC inhibitors (carbidopa and benserazide) and preferred COMT inhibitors (entacapone, nitecapone and tolcapone).

FIG. 2 illustrates catechol protection strategies applicable to L-DOPA and carbidopa bile acid conjugates.

FIG. 3 illustrates multi-drug bile acid conjugates for sustained release of L-DOPA, wherein Y and Y′ are optional linker groups, D and D′ are independently L-DOPA, carbidopa, benserazide, entacapone, nitecapone and tolcapone, but at least one of D and D′ is L-DOPA.

FIGS. 4-10 illustrate bile acid conjugates for sustained release of L-DOPA.

FIGS. 11-18 illustrate bile acid conjugates for sustained release of carbidopa.

FIGS. 19-23 illustrate bile acid conjugates for sustained release of benerazide.

FIGS. 24 and 25 illustrate bile acid conjugates for sustained release of the COMT inhibitors.

FIGS. 26-28 illustrate a method of preparing some intermediates for the preparation of some of the compounds of formula (I) .

FIGS. 29-31 illustrate the preparation of some of the compounds of formula (I) where D is L-DOPA or carbidopa with D linked to Y via an ester linkage obtained via a reaction of the carboxyl group of L-DOPA or carbidopa.

FIG. 32 illustrates a method for preparing some of the compounds of formula (I) where D is L-DOPA or carbidopa with D linked to Y via an amide linkage obtained via a reaction of the carboxyl group of L-DOPA or carbidopa.

FIGS. 33-35 illustrate the preparation of some of the compounds of formula (I) where D is L-DOPA, carbidopa or benserazide with D linked to Y via an amide linkage obtained by a reaction of an amino group of D.

FIGS. 36 and 37 illustrate the preparation of some of the compounds of formula (I) where D is L-DOPA, carbidopa, benserazide, entacapone, nitecapone or tolcapone with D linked to Y via a hydroxyl group of D.

FIG. 38 illustrates another method for preparing some of the compounds of formula (I) where D is L-DOPA or carbidopa with D linked to Y via an amide linkage obtained via a reaction of the carboxyl group of L-DOPA or carbidopa.

FIG. 39 illustrates the synthetic scheme used to synthesis a catechol protected L-Dopa derivative conjugated to the C-24 position of cholic acid by formation of an amide bond.

FIG. 40 illustrates the synthetic scheme used to prepare L-Dopa-containing dipeptides conjugated to the C-24 position of cholic acid by formation of an amide bond.

FIG. 41 illustrates the synthetic scheme used to prepare esters of L-Dopa conjugated to the C-24 position of cholic acid by formation of an amide bond.

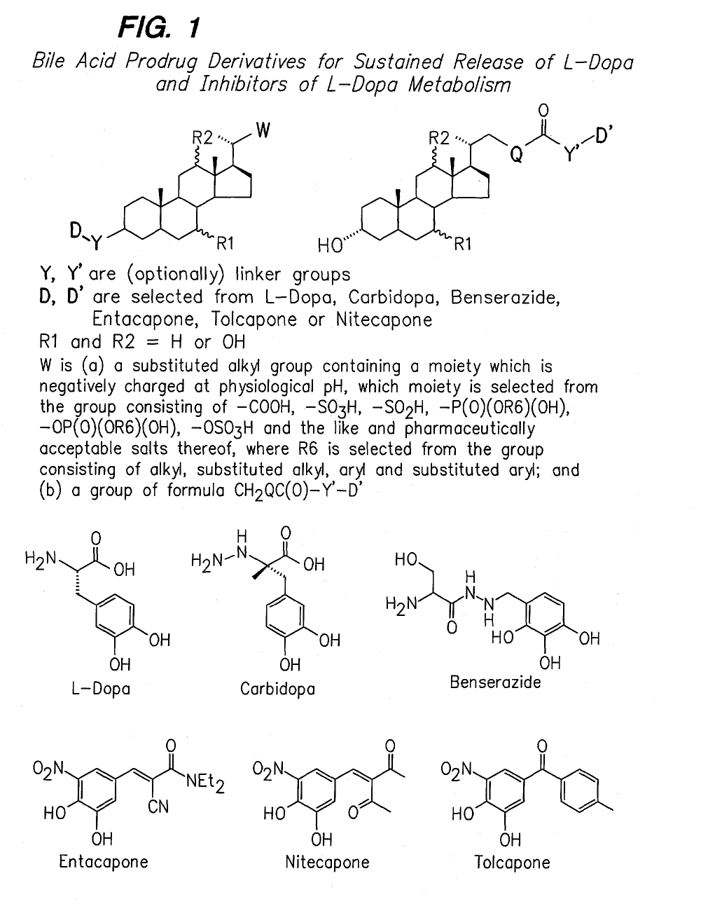

### DETAILED DESCRIPTION OF THE INVENTION

This invention provides compositions and methods for providing sustained release of levodopa, the AADC inhibitors, e.g. carbidopa and benserazide, and/or the COMT inhibitors, e.g. entacapone, nitecapone and tolcapone. Specifically, such compounds are reversibly coupled to a compound capable of undergoing absorption across the intestinal epithelium and enterohepatic recirculation via active transport through the bile acid transport system. Cleavage of the drug from a portion of the total conjugate present during each cycle through the enterohepatic circulation provides for sustained release of the drug.

However, prior to describing this invention in further detail, the following terms will first be defined:

### Definitions

As used herein, the term “translocation across the intestinal wall” refers to movement of a drug or drug conjugate by a passive or active mechanism, or both, across an epithelial cell membrane of any region of the gastrointestinal tract.

“Active metabolite of a drug” refers to products of in vivo modification of the compounds of this invention which have therapeutic or prophylactic effect.

“Therapeutic or prophylactic blood concentrations” refers to systemic exposure to a sufficient concentration of a drug or an active metabolite thereof over a sufficient period of time to effect disease therapy or to prevent the onset or reduce the severity of a disease in the treated animal.

“Sustained release” refers to release of a therapeutic or prophylactic amount of the drug or an active metabolite thereof into the systemic blood circulation over a prolonged period of time relative to that achieved by oral administration of a conventional formulation of the drug.

“Tissue of the enterohepatic circulation” refers to the blood, plasma, intestinal contents, intestinal cells, liver cells, biliary tract or any fraction, suspension, homogenate, extract or preparation thereof.

“Conjugating” refers to the formation of a covalent bond.

“Bile acid transport system” refers to any membrane transporter protein capable of causing a bile acid or a derivative thereof to be translocated across a membrane of a cell of the gastrointestinal tract or liver.

“Active transport or active transport mechanism” refers to the movement of molecules across cellular membranes that:

a) is directly or indirectly dependent on an energy mediated process (i.e., driven by ATP hydrolysis, ion gradient, etc); or

b) occurs by facilitated diffusion mediated by interaction with specific transporter proteins; or

c) occurs through a modulated solute channel.

“A moiety selected to permit a compound of formula (i) to be translocated across the intestinal wall of an animal via the bile acid transport system” or “a compound which is a substrate for an intestinal bile acid transporter” refers to compounds which, when conjugated to the drug/cleavable linker moiety, are translocated across the intestinal wall via the bile acid transport system. Evaluation of which candidate compounds can be so translocated across the intestinal wall can be conducted by the in vitro assay set forth in Example 5 below.

“Treating” a particular disease or disorder means reducing the number of symptoms and/or severity of symptoms of the disease, and/or reducing or limiting the further progression of the disease.

“Preventing” a disease or disorder means preventing or inhibiting the onset or occurrence of the disease or disorder.

“Cleavable linker” refers to linkers Y and Y′ that contain one or more functional groups that permit cleavage of such linkers in vivo by, for example, endogenous enzymes, such as esterases and amidases. Preferably, the functional group subject to cleavage in the cleavable linker is attached adjacent the moiety, D or D′, such that upon cleavage, free L-DOPA, a free L-DOPA derivative, a catechol O-methyl transferase inhibitor, or an L-acromatic amino acid decarboxylase inhibitor is released. The cleavable linker preferably comprises one or more functional groups such as ester groups, amide groups, glycolamide ester groups, amidomethyl esters, acyloxyalkyl esters, alkoxycarbonyloxyalkyl esters, and the like.

“Derivatives of L-DOPA” preferably refers to L-DOPA molecules wherein:

a) a hydrogen atom of the amino group of the L-DOPA molecule is replaced with —C(O)R⁴, —C(O)OR⁵ or an amino acid group, wherein R⁴ is selected from the group consisting of hydrogen, alkyl, substituted alkyl, cycloalkyl, substituted cycloalkyl, aryl, substituted aryl, aralkyl, substituted aralkyl, heteroaryl and substituted heteroaryl, and R⁵ is selected from the group consisting of alkyl, substituted alkyl, cycloalkyl, substituted cycloalkyl, aryl, substituted aryl, aralkyl, substituted aralkyl, heteroaryl and substituted heteroaryl; and/or

b) one or two hydrogen atoms of the two —OH groups of the catechol group of the L-DOPA molecule are replaced with —C(O)R⁴, —C(O)OR⁵ and/or —OCR³R⁴OC(O)R⁵ wherein R⁵ is defined as above, R³ and R⁴ independently are members selected from the group consisting of hydrogen, alkyl, substituted alkyl, cycloalkyl, substituted cycloalkyl, aryl, substituted aryl, aralkyl, substituted aralkyl, heteroaryl and substituted heteroaryl, or R³ and R⁴ together with the carbon atom to which they are attached form a cycloalkyl, substituted cycloalkyl, heterocycle or substituted heterocyclic ring, or the two —OH groups of the catechol group of the L-DOPA molecule are protected with a 5-membered cyclic carbonate or 2,3-dioxo-1,4-dioxane ortho fused with a benzene ring of the catechol group of the L-DOPA molecule; and/or

c) the OH group of the carboxyl moiety is replaced by —OR⁴ with the proviso that one of the amino hydrogen atoms, the hydroxyl group of the carboxyl moiety or the hydrogen atom of one of the hydroxyl groups of the catechol is removed to form a covalent bond to Y or Y′.

“An inhibitor of L-aromatic amino acid decarboxylase” preferably refers to L-aromatic amino acid decarboxylase inhibitors such as carbidopa and benzserazide optionally with a hydrogen atom of the amino or the hydrazido group of the L-aromatic amino acid decarboxylase inhibitor replaced with —C(O)R⁴, —C(O)OR⁵ or an amino acid group, wherein R⁴ is selected from the group consisting of hydrogen, alkyl, substituted alkyl, cycloalkyl, substituted cycloalkyl, aryl, substituted aryl, aralkyl, substituted aralkyl, heteroaryl and substituted heteroaryl, and R⁵ is selected from the group consisting of alkyl, substituted alkyl, cycloalkyl, substituted cycloalkyl, aryl, substituted aryl, aralkyl, substituted aralkyl, heteroaryl and substituted heteroaryl; and/or

optionally with one or two hydrogen atoms of the two —OH groups of the catechol or the three —OH groups of the pyrogallol group of the L-aromatic amino acid decarboxylase inhibitor are replaced with —C(O)R⁴, —C(O)OR⁵ and/or —OCR³R⁴OC(O)R⁵ wherein R⁵ is defined as above, R³ and R⁴ independently are members selected from the group consisting of hydrogen, alkyl, substituted alkyl, cycloalkyl, substituted cycloalkyl, aryl, substituted aryl, aralkyl, substituted aralkyl, heteroaryl and substituted heteroaryl, or R³ and R⁴ together with the carbon atom to which they are attached form a cycloalkyl, substituted cycloalkyl, heterocycle or substituted heterocyclic ring; or optionally with two adjacent —OH groups of the catechol or pyrogallol group protected with a 5-membered cyclic carbonate or 2,3-dioxo-1,4-dioxane ortho fused with a benzene ring of the catechol or pyrogallol group; and/or

the OH group of the carboxyl moiety is replaced by —OR⁴ with the proviso that one of the amino hydrogen atoms, the hydroxyl group of the carboxyl moiety or the hydrogen atom of one of the hydroxyl groups of the catechol/pyrogallol is removed to form a covalent bond to Y or Y′.

“Catechol O-methyl transferase inhibitor” preferably refers to catechol O-methyl transferase inhibitors such as entacapone, nitecapone and tolcapone optionally with one or two hydrogen atoms of two hydroxyl groups of the catechol group replaced with —C(O)R⁴, —C(O)OR⁵ and/or —OCR³R⁴OC(O)R⁵, wherein R³ and R⁴ independently are members selected from the group consisting of hydrogen, alkyl, substituted alkyl, cycloalkyl, substituted cycloalkyl, aryl, substituted aryl, aralkyl, substituted aralkyl, heteroaryl and substituted heteroaryl, or R³ and R⁴ together with the carbon atom to which they are attached form a cycloalkyl, substituted cycloalkyl, heterocycle or substituted heterocyclic ring, R⁵ is selected from the group consisting of alkyl, substituted alkyl, cycloalkyl, substituted cycloalkyl, aryl, substituted aryl, aralkyl, substituted aralkyl, heteroaryl and substituted heteroaryl, or the OH group of the carboxyl moiety is replaced by —OR⁴

with the proviso that one of the amino hydrogen atoms or the hydrogen atom of one of the hydroxyl groups of the catechol is removed to form a covalent bond to Y or Y′. “Steroid” or “sterol” refers to the following core structure with the appropriate numbering system inserted therein:

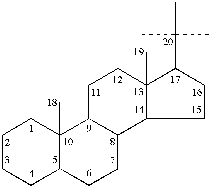

Accordingly, cholic acid which has the structure:

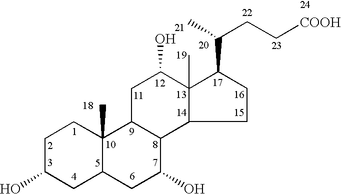

is numbered as shown above.

“Alkyl” refers to alkyl groups preferably having from 1 to 20 carbon atoms and more preferably 1 to 6 carbon atoms. This term is exemplified by groups such as methyl, t-butyl, n-heptyl, octyl, dodecyl and the like. Alkyl groups having from 1 to 6 carbon atoms are also termed “lower alkyl” groups.

“Substituted alkyl” refers to an alkyl group, preferably of from 1 to 20 carbon atoms, having from 1 to 5 substituents selected from the group consisting of alkoxy, substituted alkoxy, acyl, acylamino, thiocarbonylamino, acyloxy, amino, amidino, alkyl amidino, thioamidino, aminoacyl, aminocarbonylamino, aminothiocarbonylamino, aminocarbonyloxy, aryl, substituted aryl, aryloxy, substituted aryloxy, aryloxylaryl, substituted aryloxyaryl, cyano, halogen, hydroxyl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted aryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR—alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclicamino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and substituted alkyl groups having amino groups blocked by conventional blocking groups such as Boc, Cbz, formyl, and the like or alkyl/substituted alkyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkoxy” refers to the group “alkyl-O—” which includes, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

“Substituted alkoxy” refers to the group “substituted alkyl-O—”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)— cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acylamino” refers to the group —C(O)NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiocarbonylamino” refers to the group —C(S)NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form, together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Alkenyl” refers to alkenyl group preferably having from 2 to 20 carbon atoms and more preferably 2 to 6 carbon atoms and having at least 1 and preferably from 1-2 sites of alkenyl unsaturation.

“Substituted alkenyl” refers to alkenyl groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic, substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkynyl” refers to alkynyl group preferably having from 2 to 20 carbon atoms and more preferably 3 to 6 carbon atoms and having at least 1 and preferably from 1-2 sites of alkynyl unsaturation.

“Substituted alkynyl” refers to alkynyl groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic, substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkylene” refers to a divalent alkylene group preferably having from 1 to 20 carbon atoms and more preferably 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Substituted alkylene” refers to alkylene groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic, substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkenylene” refers to a divalent alkenylene group preferably having from 2 to 20 carbon atoms and more preferably 1 to 6 carbon atoms and having from 1 to 2 sites of alkenyl unsaturation. This term is exemplified by groups such as ethenylene (—CH═CH—), propenylene (—CH₂CH═CH—), and the like.

“Substituted alkenylene” refers to alkenylene groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic, substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Alkynylene” refers to a divalent alkynylene group preferably having from 2 to 20 carbon atoms and more preferably 1 to 6 carbon atoms and having from 1 to 2 sites of alkynyl unsaturation. This term is exemplified by groups such as ethynylene, propynylene and the like.

“Substituted alkynylene” refers to alkynylene groups having from 1 to 5 substituents selected from the group consisting of alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic, substituted heterocyclic and substituted alkenyl groups having amino groups blocked by conventional blocking groups such as Boc, Cbz, formyl, and the like or alkenyl/substituted alkenyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Amidino” refers to the group H₂NC(═NH)— and the term “alkylamidino” refers to compounds having 1 to 3 alkyl groups (e.g., alkylHNC(═NH)—).

“Thioamidino“ refers to the group RSC(═NH)— where R is hydrogen or alkyl.

“Aminoacyl” refers to the groups —NRC(O)alkyl, —NRC(O)substituted alkyl, —NRC(O)cycloalkyl, —NRC(O)substituted cycloalkyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” refers to the groups —NRC(O)O-alkyl, —NRC(O)O-substituted alkyl, —NRC(O)O-alkenyl, —NRC(O)O-substituted alkenyl, —NRC(O)O-alkynyl, —NRC(O)O-substituted alkynyl, —NRC(O)O-cycloalkyl, —NRC(O)O-substituted cycloalkyl, —NRC(O)O-aryl, —NRC(O)O-substituted aryl, —NRC(O)O-heteroaryl, —NRC(O)O-substituted heteroaryl, —NRC(O)O-heterocyclic, and —NRC(O)O-substituted heterocyclic where R is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxycarbonylamino” refers to the groups —OC(O)NH₂, —OC(O)NRR, —OC(O)NR-alkyl, —OC(O)NR-substituted alkyl, —OC(O)NR-alkenyl, —OC(O)NR-substituted alkenyl, —OC(O)NR-alkynyl, —OC(O)NR-substituted alkynyl, —OC(O)NR-cycloalkyl, —OC(O)NR-substituted cycloalkyl, —OC(O)NR-aryl, —OC(O)NR-substituted aryl, —OC(O)NR-heteroaryl, —OC(O)NR-substituted heteroaryl, —OC(O)NR-heterocyclic, and —OC(O)NR-substituted heterocyclic where R is hydrogen, alkyl or where each R is joined to form, together with the nitrogen atom a heterocyclic or substituted heterocyclic ring and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Oxythiocarbonylamino” refers to the groups —OC(S)NH₂, —OC(S)NRR, —OC(S)NR-alkyl, —OC(S)NR-substituted alkyl, —OC(S)NR-alkenyl, —OC(S)NR-substituted alkenyl, —OC(S)NR-alkynyl, —OC(S)NR-substituted alkynyl, —OC(S)NR-cycloalkyl, —OC(S)NR-substituted cycloalkyl, —OC(S)NR-aryl, —OC(S)NR-substituted aryl, —OC(S)NR-heteroaryl, —OC(S)NR-substituted heteroaryl, —OC(S)NR-heterocyclic, and —OC(S)NR-substituted heterocyclic where R is hydrogen, alkyl or where each R is joined to form together with the nitrogen atom a heterocyclic or substituted heterocyclic ring and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the groups —NRC(O)NRR, —NRC(O)NR-alkyl, —NRC(O)NR-substituted alkyl, —NRC(O)NR-alkenyl, —NRC(O)NR-substituted alkenyl, —NRC(O)NR-alkynyl, —NRC(O)NR-substituted alkynyl, —NRC(O)NR-aryl, —NRC(O)NR-substituted aryl, —NRC(O)NR-cycloalkyl, —NRC(O)NR-substituted cycloalkyl, —NRC(O)NR-heteroaryl, and —NRC(O)NR-substituted heteroaryl, —NRC(O)NR-heterocyclic, and —NRC(O)NR-substituted heterocyclic where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonylamino” refers to the groups —NRC(S)NRR, —NRC(S)NR-alkyl, —NRC(S)NR-substituted alkyl, —NRC(S)NR-alkenyl, —NRC(S)NR-substituted alkenyl, —NRC(S)NR-alkynyl, —NRC(S)NR-substituted alkynyl, —NRC(S)NR-aryl, —NRC(S)NR-substituted aryl, —NRC(S)NR-cycloalkyl, —NRC(S)NR-substituted cycloalkyl, —NRC(S)NR-heteroaryl, and —NRC(S)NR-substituted heteroaryl, —NRC(S)NR-heterocyclic, and —NRC(S)NR-substituted heterocyclic where each R is independently hydrogen, alkyl or where each R is joined to form together with the nitrogen atom a heterocyclic or substituted heterocyclic ring as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aryl” or “Ar” refers to a monovalent unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7yl, and the like). Preferred aryls include phenyl and naphthyl.

“Substituted aryl” refers to aryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Arylene” refers to a divalent unsaturated aromatic carbocyclic group of from 6 to 14 carbon atoms having a single ring (e.g., phenylene) or multiple condensed rings (e.g., naphthylene or anthrylene) which condensed rings may or may not be aromatic. Preferred arylenes include phenylene and naphthylene.

“Substituted arylene” refers to arylene groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Aryloxy” refers to the group aryl-O— which includes, by way of example, phenoxy, naphthoxy, and the like.

“Substituted aryloxy” refers to substituted aryl-O— groups.

“Aryloxyaryl” refers to the group -aryl-O-aryl.

“Substituted aryloxyaryl” refers to aryloxyaryl groups substituted with from 1 to 3 substituents on either or both aryl rings selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamnino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Alkaryl” refers to the groups -alkylene aryl and -subsituted alkylene aryl wherein alkylene, substituted alkylene and aryl are as defined herein and are exemplified by groups such as benzyl, phenethyl and the like.

“Cycloalkyl” refers to cyclic alkyl groups of from 3 to 8 carbon atoms having a single cyclic ring including, by way of example, cyclopropyl, cyclobutyl, cyclopentyl, cyclooctyl and the like. Excluded from this definition are multi-ring alkyl groups such as adamantanyl, etc.

“Cycloalkenyl” refers to cyclic alkenyl groups of frm 3 to 8 carbon atoms having a single cyclic ring.

“Substituted cycloalkyl” and “substituted cycloalkenyl” refers to an cycloalkyl or cycloalkenyl group, preferably of from 3 to 8 carbon atoms, having from 1 to 5 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Cycloalkylene” refers to divalent cyclic alkylene groups of from 3 to 8 carbon atoms having a single cyclic ring including, by way of example, cyclopropylene, cyclobutylene, cyclopentylene, cyclooctylene and the like.

“Cycloalkenylene” refers to a divalent cyclic alkenylene groups of from 3 to 8 carbon atoms having a single cyclic ring.

“Substituted cycloalkylene” and “substituted cycloalkenylene” refers to a cycloalkylene or cycloalkenylene group, preferably of from 3 to 8 carbon atoms, having from 1 to 5 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino , mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Cycloalkoxy” refers to —O-cycloalkyl groups.

“Substituted cycloalkoxy” refers to —O-substituted cycloalkyl groups.

“Hydrocarbyl radical” is a moiety containing only carbon and hydrogen atoms, characterized by alkylene, alkenylene, alkynylene, cycloalkylene, cycloalkenylene, arylene, alkarylene, and the like.

“Guanidino” refers to the groups —NRC(═NR)NRR, —NRC(═NR)NR-alkyl, —NRC(═NR)NR-substituted alkyl, —NRC(═NR)NR-alkenyl, —NRC(═NR)NR-substituted alkenyl, —NRC(═NR)NR-alkynyl, —NRC(═NR)NR-substituted alkynyl, —NRC(═NR)NR-aryl, —NRC(═NR)NR-substituted aryl, —NRC(═NR)NR-cycloalkyl, —NRC(═NR)NR-heteroaryl, —NRC(═NR)NR-substituted heteroaryl, —NRC(═NR)NR-heterocyclic, and —NRC(═NR)NR-substituted heterocyclic where each R is independently hydrogen and alkyl as well as where one of the amino groups is blocked by conventional blocking groups such as Boc, Cbz, formyl, and the like and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“N,N-Dinethylcarbamyloxy” refers to the group —OC(O)N(CH₃)₂.

“Guanidinosulfone” refers to the groups —NRC(═NR)NRSO₂-alkyl, —NRC(═NR)NRSO₂-substituted alkyl, —NRC(═NR)NRS₂-alkenyl, —NRC(═NR)NRSO₂-substituted alkenyl, —NRC(═NR)NRSO₂-alkynyl, —NRC(═NR)NRSO₂-substituted alkynyl, —NRC(═NR)NRSO₂-aryl, —NRC(═NR)NRSO₂-substituted aryl, —NRC(═NR)NRSO₂-cycloalkyl, —NRC(═NR)NRSO₂-substituted cycloalkyl, —NRC(═NR)NRSO₂-heteroaryl, and —NRC(═NR)NRSO₂-substituted heteroaryl, —NRC(═NR)NRSO₂-heterocyclic, and —NRC(═NR)NRSO₂-substituted heterocyclic where each R is independently hydrogen and alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Halo” or “halogen” refers to fluoro, chloro, bromo and iodo and preferably is either chloro or bromo.

“Heteroaryl” refers to an aromatic carbocyclic group of from 2 to 10 carbon atoms and 1 to 4 heteroatoms selected from the group consisting of oxygen, nitrogen and sulfur within the ring. Such heteroaryl groups can have a single ring (e.g., pyridyl or furyl) or multiple condensed rings (e.g. indolizinyl or benzothienyl). Preferred heteroaryls include pyridyl, pyrrolyl, indolyl and furyl.

“Substituted heteroaryl” refers to heteroaryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Heteroarylene” refers to a divalent aromatic carbocyclic group of from 2 to 10 carbon atoms and 1 to 4 heteroatoms selected from the group consisting of oxygen, nitrogen and sulfur within the ring. Such heteroarylene groups can have a single ring (e.g., pyridylene or furylene) or multiple condensed rings (e.g., indolizinylene or benzothienylene). Preferred heteroarylenes include pyridylene, pyrrolylene, indolylene and furylene.

“Substituted heteroarylene” refers to heteroarylene groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Heteroaryloxy” refers to the group —O-heteroaryl and “substituted heteroaryloxy” refers to the group —O-substituted heteroaryl.

“Heterocycle” or “heterocyclic” refers to a saturated or unsaturated group having a single ring or multiple condensed rings, from 1 to 10 carbon atoms and from 1 to 4 hetero atoms selected from the group consisting of nitrogen, sulfur or oxygen within the ring wherein, in fused ring systems, one or more the rings can be aryl or heteroaryl.

“Substituted heterocyclic” refers to heterocycle groups which are substituted with from 1 to 3 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, —C(O)O-aryl, —C(O)O-substituted aryl, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclene” refers to a divalent saturated or unsaturated group having a single ring or multiple condensed rings, from 1 to 10 carbon atoms and from 1 to 4 hetero atoms selected from the group consisting of nitrogen, sulfur or oxygen within the ring wherein, in fused ring systems, one or more the rings can be aryl or heteroaryl.

“Substituted heterocyclene” refers to heterocyclene groups which are substituted with from 1 to 3 substituents selected from the group consisting of oxo (═O), thioxo (═S), alkoxy, substituted alkoxy, acyl, acylamino, thiocarbonylamino, acyloxy, amino, amidino, alkylamidino, thioamidino, aminoacyl, aminocarbonylamino, aminothiocarbonylamino, aminocarbonyloxy, aryl, substituted aryl, aryloxy, substituted aryloxy, aryloxyaryl, substituted aryloxyaryl, halogen, hydroxyl, cyano, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, substituted cycloalkyl, guanidino, guanidinosulfone, thiol, thioalkyl, substituted thioalkyl, thioaryl, substituted thioaryl, thiocycloalkyl, substituted thiocycloalkyl, thioheteroaryl, substituted thioheteroaryl, thioheterocyclic, substituted thioheterocyclic, heteroaryl, substituted heteroaryl, heterocyclic, substituted heterocyclic, cycloalkoxy, substituted cycloalkoxy, heteroaryloxy, substituted heteroaryloxy, —C(O)O-aryl, —C(O)O-substituted aryl, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents selected from the group consisting of alkyl, substituted alkyl, aryl, substituted aryl, heteroaryl, substituted heteroaryl, heterocyclic and substituted heterocyclic and substituted alkynyl groups having amino groups blocked by conventional blocking groups such as Boc, Cbz, formyl, and the like or alkynyl/substituted alkynyl groups substituted with —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic and —SO₂NRR where R is hydrogen or alkyl.

“Heterocyclyloxy” refers to the group —O-heterocyclic and “substituted heterocyclyloxy” refers to the group —O-substituted heterocyclic.

“Thiol” refers to the group —SH.

“Thioalkyl” refers to the group —S-alkyl.

“Substituted thioalkyl” refers to the group —S-substituted alkyl.

“Thiocycloalkyl” refers to the group —S-cycloalkyl.

“Substituted thiocycloalkyl” refers to the group —S-substituted cycloalkyl.

“Thioaryl” refers to the group —S-aryl and “substituted thioaryl” refers to the group —S-substituted aryl.

“Thioheteroaryl” refers to the group —S-heteroaryl and “substituted thioheteroaryl” refers to the group —S-substituted heteroaryl.

“Thioheterocyclic” refers to the group —S-heterocyclic and “substituted thioheterocyclic” refers to the group —S-substituted heterocyclic.

It is understood, of course, that combinations of substituents within the compounds of formula (i) above do not include any combination which is chemical impossible or non-feasible as would be appreciated by one skilled in the art.

“Pharmaceutically acceptable salt” refers to pharmaceutically acceptable salts of a compound of formula (i) which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

### Preferred Compounds

Preferred compounds of the present invention are compounds represented by formula (I-a):

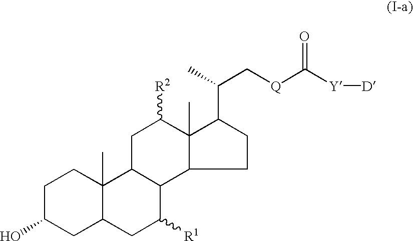

wherein:

Y′ is selected from the group consisting of a covalent bond and a cleavable linker group covalently connecting D′ to the C-24 position of the steroid;

D′ is a member selected from the group consisting of L-DOPA, a catechol O-methyl transferase inhibitor, an inhibitor of a L-aromatic amino acid decarboxylase, and derivatives of L-DOPA;

Q is CH₂ or O;

R¹ and R² are one of the following combinations:

R¹ and R² are α-OH;

R¹ is α-OH and R² is H;

R¹ is β-OH and R² is H;

R¹ is H and R² is α-OH;

R¹ is β-OH and R² is α-OH; or

R¹ and R² are H;

wherein the compound of formula (I-a) above is a substrate for an intestinal bile acid transporter;

or pharmaceutically acceptable salts thereof.

Particularly preferred prodrugs of formula (I-a) are compounds represented by formulae (I-a-1) and (I-a-2):

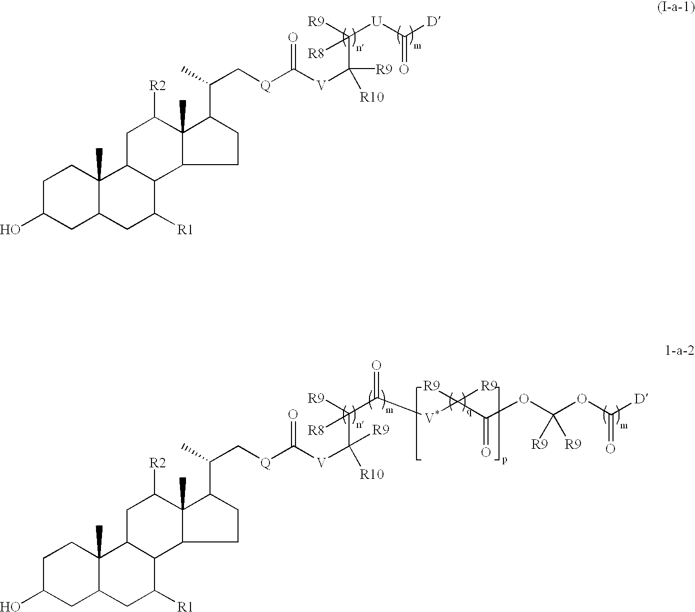

wherein:

D′ is a member selected from the group consisting of L-DOPA, a catechol O-methyl transferase inhibitor, an inhibitor of a L-aromatic amino acid decarboxylase, and derivatives of L-DOPA;

Q is CH₂ or O;

R¹ and R² are one of the following combinations:

R¹ and R² are α-OH;

R¹ is α-OH and R² is H;

R¹ is β-OH and R ² is H;

R¹ is H and R² is α-OH;

R¹ is β-OH and R² α-OH; or

R¹ and R² are H;

V and V^{*} are independently NR⁷, O, S or CR⁸R⁹;

U is NR⁷, O, S;

R¹⁰ is R⁸ or (CR⁸R⁹)ᵣ,T;

T is selected from the group consisting of CO₂H, SO₃H, OSO₃H, SO₂H, P(O)(OR⁶)(OH), OP(O)(OR⁶)(OH) and pharmaceutically acceptable salts thereof;

each m is 0 or 1;

n′ is 0, 1, 2, 3 or 4;

p is 0, 1,2 ,3 ,4, 5, or 6;

each q is independently 1, 2, 3, 4, 5, or 6;

r is 0 or 1;

R⁶ is selected from the group consisting of alkyl, substituted alkyl, aryl and substituted aryl;

R⁷, R⁸ and R⁹ are independently hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or, when R⁷ and R⁹ are present and attached to adjacent atoms, then R⁷ and R⁹ together with the atoms to which they are attached form a cycloalkyl, substituted cycloalkyl, heterocycle or substituted heterocyclic ring;

R¹¹ and R¹² are independently hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring;

wherein the compound of formulae (I-a-1) and (I-a-2) above is a substrate for an intestinal bile acid transporter;

or pharmaceutically acceptable salts thereof.

Another preferred group of prodrugs of the present invention are compounds represented by formula (I-b):

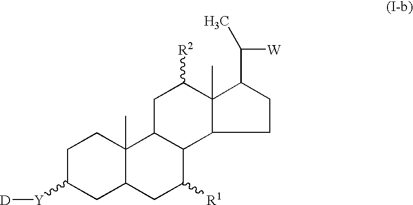

wherein:

Y is selected from the group consisting of a covalent bond and a cleavable linker group covalently connecting D to the steroid;

D is a member selected from the group consisting of L-DOPA, a catechol O-methyl transferase inhibitor, an inhibitor of a L-aromatic amino acid decarboxylase, and derivatives of L-DOPA;

R¹ and R² are one of the following combinations:

R¹ and R² are α-OH;

R is α-OH and R² is H;

R¹ is β-OH and R² is H;

R¹ is H and R² is α-OH;

R¹ is β-OH and R² is α-OH; or

R¹ and R² are H;

W is a substituted alkyl group containing a moiety which is negatively charged at physiological pH, which moiety is selected from the group consisting of —COOH, —SO₃H, —SO₂H, —P(O)(OR⁶)(OH), —OP(O)(OR⁶)(OH), —OSO₃H and the like and pharmaceutically acceptable salts thereof, where R⁶ is selected from the group consisting of alkyl, substituted alkyl, aryl and substituted aryl;

wherein the compound of formula (I-b) above is a substrate for an intestinal bile acid transporter;

or pharmaceutically acceptable salts thereof

Particularly preferred examples of suitable cleavable linkers Y for use in formula (I-b) include structures of formulae (i) through (v) as shown below;

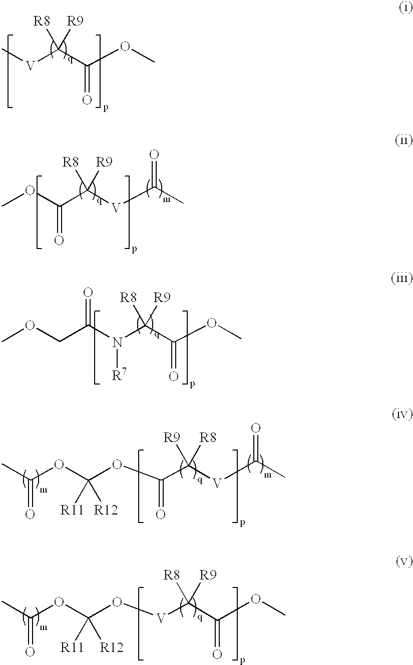

wherein

V is selected from the group consisting of NR⁷, O, S and CR⁸R⁹;

each m is independently 0 or 1;

p is 0, 1,2 ,3 4, 5, or 6;

q is 1, 2, 3, 4, 5or 6;

each R⁷, R⁸ and R⁹ is independently hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or, when R⁷ and R⁹ are present and attached to adjacent atoms, then R⁷ and R⁹ together with the atoms to which they are attached form a cycloalkyl, substituted cycloalkyl, heterocycle or substituted heterocyclic ring;

R¹¹ and R¹² are independently hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring.

Still another preferred group of prodrugs of the present invention are compounds represented by formula (I-c):

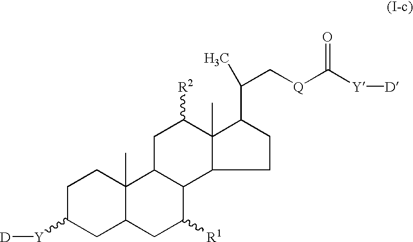

wherein:

Y′ is selected from the group consisting of a covalent bond and a cleavable linker group covalently connecting D′ to the C-24 position of the steroid;

D′ is a member selected from the group consisting of L-DOPA, a catechol O-methyl transferase inhibitor, an inhibitor of a L-aromatic amino acid decarboxylase, and derivatives of L-DOPA;

Y is selected from the group consisting of a covalent bond and a cleavable linker group covalently connecting D to the steroid;

D is a member selected from the group consisting of L-DOPA, a catechol O-methyl transferase inhibitor, an inhibitor of a L-aromatic amino acid decarboxylase, and derivatives of L-DOPA;

Q is CH₂ or O;

R¹ and R² are one of the following combinations:

R¹ and R² are α-OH;

R¹ is α-OH and R² is H;

R¹ is β-OH and R² is H;

R¹ is H and R² is α-OH;

R¹ is β-OH and R² is α-OH; or

R¹ and R² are H;

wherein the compound of formula (I-c) above is a substrate for an intestinal bile acid transporter;

or pharmaceutically acceptable salts thereof.

Particularly preferred prodrugs of formula (I-c) are compounds represented by formulae (I-c-1) and (I-c-2):

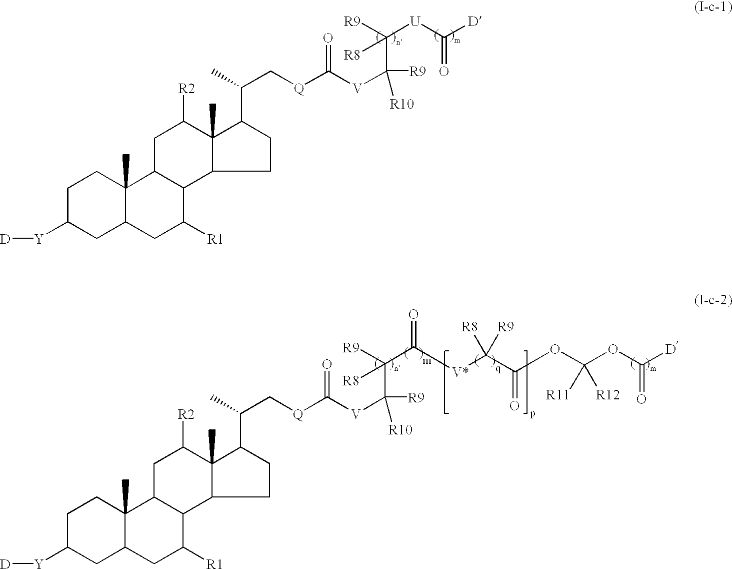

wherein:

D′ is a member selected from the group consisting of L-DOPA, a catechol O-methyl transferase inhibitor, an inhibitor of a L-aromatic amino acid decarboxylase, and derivatives of L-DOPA;

D is a member selected from the group consisting of L-DOPA, a catechol O-methyl transferase inhibitor, an inhibitor of a L-aromatic amino acid decarboxylase, and derivatives of L-DOPA;

Q is CH₂ or O;

R¹ and R² are one of the following combinations:

R¹ and R² are α-OH;

R¹ is α-OH and R² is H;

R¹ is β-OH and R² is H;

R¹ is H and R² is α-OH;

R¹ is β-OH and R² is α-OH; or

R¹ and R² are H;

Y is selected from the group consisting of structures of formulae (i) through (v) below:

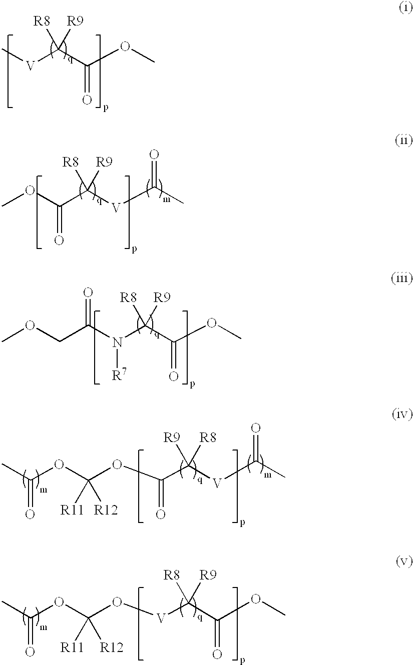

wherein

each V and V^{*} are independently NR⁷, O, S or CR⁸R⁹;

U is NR⁷, O, S;

R¹⁰ is R⁸ or (CR⁸R⁹)rT′;

T′ is selected from the group consisting of CO₂H, SO₃H, OSO₃H, SO₂H, P(O)(OR⁶)(OH), OP(O)(OR⁶)(OH) and pharmaceutically acceptable salts thereof;

each m is 0 or 1;

n′ is 0, 1, 2, 3 or 4;

p is 0, 1,2 ,3 ,4, 5, or 6;

each q is independently 1, 2, 3, 4, 5, or 6;

r is 0 or 1;

R⁶ is selected from the group consisting of alkyl, substituted alkyl, aryl and substituted aryl;

R⁷, R⁸ and R⁹ are independently hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or, when R⁷ and R⁹ are present and attached to adjacent atoms, then R⁷ and R⁹ together with the atoms to which they are attached form a cycloalkyl, substituted cycloalkyl, heterocycle or substituted heterocyclic ring;

R¹¹ and R¹² are independently hydrogen, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring; wherein the compound of formulae (I-c-1) and (I-c-2) above is a substrate for an intestinal bile acid transporter;

or pharmaceutically acceptable salts thereof.

It is also preferred that, in the compound according to formula (I), either D or D′ is L-DOPA or a derivative of L-DOPA. More preferably, X is -Y-D and D is L-DOPA or a derivative of L-DOPA.

In the compound of formula (I), it is also preferred that X is -Y-D, D is L-DOPA or a derivative of L-DOPA, and W is -M-Y′-D′ where D′ is a member selected from the group consisting of L-DOPA, a derivative of L-DOPA, the catechol O-methyl transferase inhibitor and the L-aromatic amino acid decarboxylase inhibitor.

More preferably, in the compound of formula (I), X is -Y-D, D is L-DOPA or a derivative of L-DOPA, and W is -M-Y′-D′ where D′ is L-DOPA or a derivative of L-DOPA.

Also more preferably, in the compound of formula (I), X is -Y-D, D is L-DOPA or a derivative of L-DOPA, and W is -M-Y′-D′ and D′ is the catechol O-methyl transferase inhibitor.

Also more preferably, in the compound of formula (I), X is -Y-D, D is L-DOPA or a derivative of L-DOPA, and W is -M-Y′-D′ where D′ is the L-aromatic amino acid decarboxylase inhibitor.

The present invention also includes the compound of formula (I), wherein W is M-Y′-D′ and D′ is L-DOPA or a derivative of L-DOPA.

Preferably, in the compound of formula (I), W is -M-Y′-D′ where D′ is L-DOPA or a derivative of L-DOPA, X is -Y-D and D is L-DOPA, a derivative of L-DOPA, a catechol O-methyl transferase inhibitor, a L-aromatic amino acid decarboxylase inhibitor, or a pharmaceutically acceptable salt thereof.

Also preferably, in the compound of formula (I), W is -M-Y′-D′ where D′ is L-DOPA or a derivative of L-DOPA, X is -Y-D and D is a catechol O-methyl transferase inhibitor.

Additionally, it is preferred that, in the compound of formula (I), W is -M-Y′-D′ where D′ is L-DOPA or a derivative of L-DOPA, X is -Y-D and D is a L-aromatic amino acid decarboxylase inhibitor.

Another aspect of the present invention is directed to compounds of formula (I), wherein X is -Y-D and D is a catechol O-methyl transferase inhibitor. In these compounds, W is preferred to be -M-Y′-D′ where D′ is a catechol O-methyl transferase inhibitor or a L-aromatic amino acid decarboxylase inhibitor. These compounds are useful in the treatment of Parkinsonism when co-administered with L-DOPA or a prodrug of L-DOPA.

Another aspect of the present invention is directed to compounds of formula (I), wherein W is -M-Y′-D′ where D′ is a catechol O-methyl transferase inhibitor. In these compounds, X is preferred to be -Y-D, wherein D is a catechol O-methyl transferase inhibitor or a L-aromatic amino acid decarboxylase inhibitor. These compounds are useful in the treatment of Parkinsonism when co-administered with L-DOPA or a prodrug of L-DOPA.

Another aspect of the present invention are compounds of formula (I), wherein X is -Y-D and D is a L-aromatic amino acid decarboxylase inhibitor. In these compounds, W is preferred to be -M-Y′-D′ where D′ is a catechol O-methyl transferase inhibitor or a L-aromatic amino acid decarboxylase inhibitor. These compounds are useful in the treatment of Parkinsonism when co-administered with L-DOPA or a prodrug of L-DOPA.

Another aspect of the present invention is directed to compounds of formula (I), wherein W is -M-Y′-D′ where D′ is a L-aromatic amino acid decarboxylase inhibitor. In these compounds, X is preferred to be -Y-D, wherein D is a catechol O-methyl transferase inhibitor or a L-aromatic amino acid decarboxylase inhibitor. These compounds are useful in the treatment of Parkinsonism when co-administered with L-DOPA or a prodrug of L-DOPA.

Among prodrugs of levodopa, carbidopa and benserazide contemplated by this invention are derivatives in which the terminal amino group of these drugs is blocked with an acyl or alkoxycarbonyl group. These functionalities undergo hydrolysis in vivo to liberate the parent drug, either before or after cleavage of the drug from bile acid or intervening linker moiety. Further contemplated by this invention are prodrugs of levodopa and carbidopa that initially undergo hydrolysis in vivo to liberate dipeptide or dipeptide analogs containing these drugs. Compounds IV-IX and LXIII-LXVIII, among others, are examples of such derivatives. These dipeptides provide the parent drug upon further proteolysis in vivo. Moreover, such derivatives can serve as substrates for the dipeptide transporters PEPT1 and PEPT2 localized in the intestine, kidney and brain. For dipeptide derivatives of levodopa, this may provide a higher capacity uptake pathway for delivery to the brain than the large neutral amino acid transporter utilized by levodopa itself. Note that it may not be desirable to induce transport of the AADC inhibitor carbidopa across the blood-brain barrier since it would block conversion of levodopa to dopamine within the CNS.

Also contemplated by this invention are prodrugs of formula (I) wherein X is Y-D and the carboxyl group (—COOH) of levodopa, a catechol O-methyl transferase inhibitor or a L-aromatic amino acid decarboxylase inhibitor is protected as an ester or an acyloxyalkyl ester.

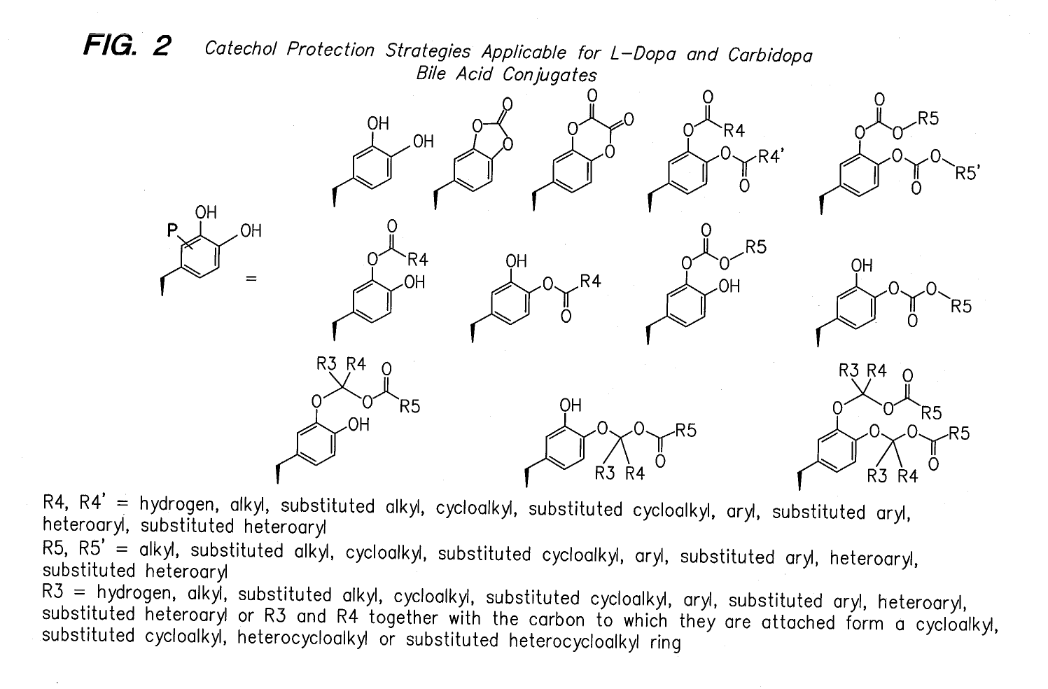

One or more of the phenolic hydroxyl groups of these prodrugs may be protected via acylation or alkylation as illustrated in FIG. 2. The corresponding ester, acyloxyalkyl ester or carbonate derivatives are hydrolyzed in vivo to regenerate the catechol moieties of the parent drugs. Such protection may be necessary, particularly for compounds having such phenolic hydroxyl groups in W, in order to permit the compounds of formula (i) to be a substrate for an intestinal bile acid transporter.

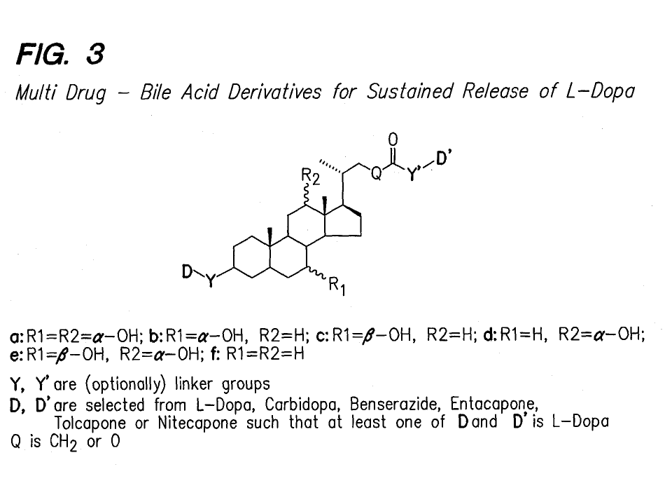

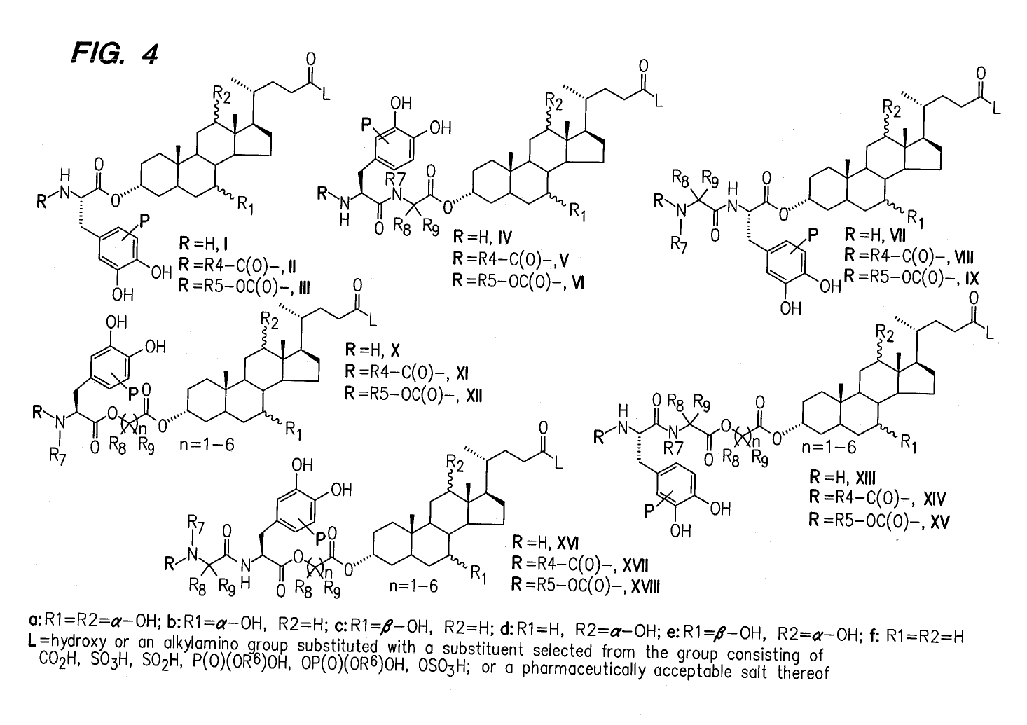

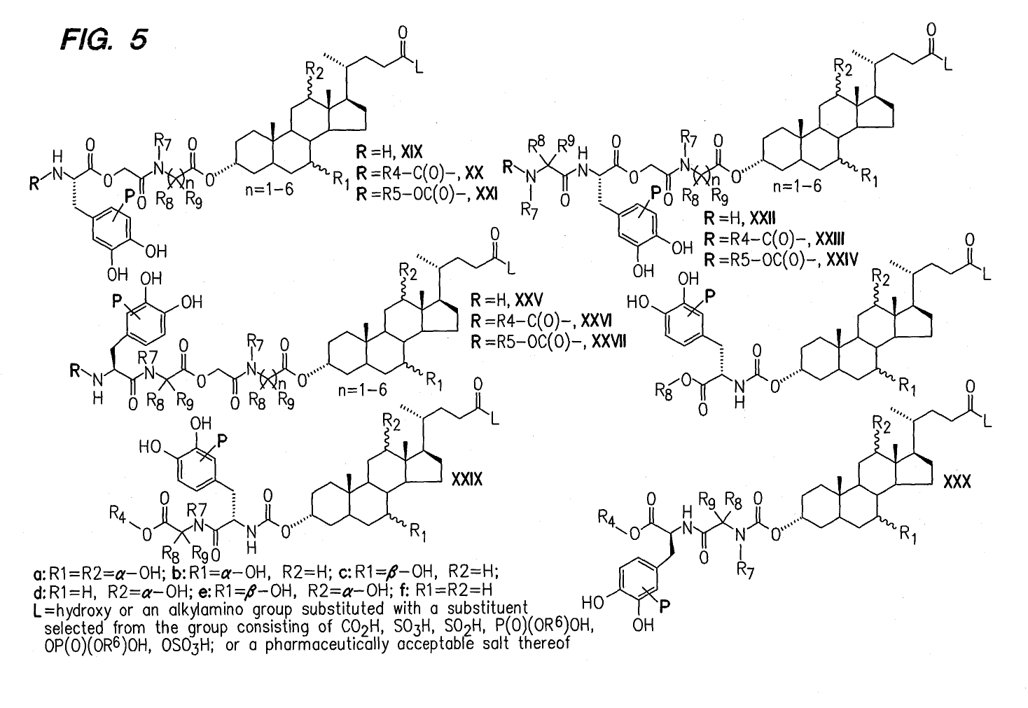

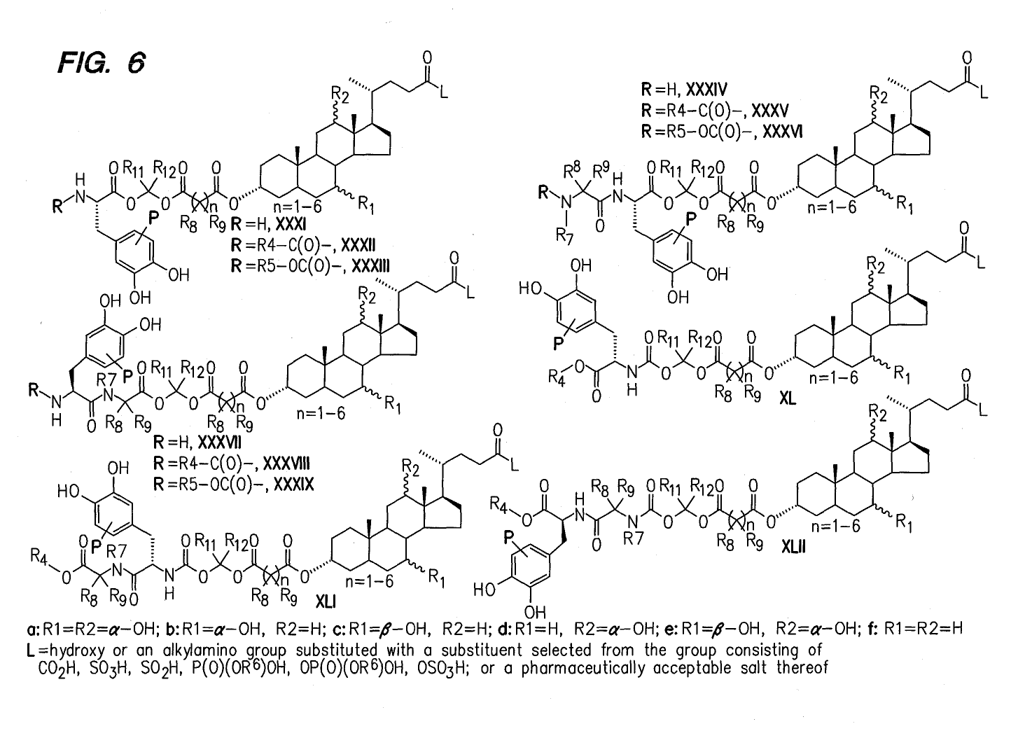

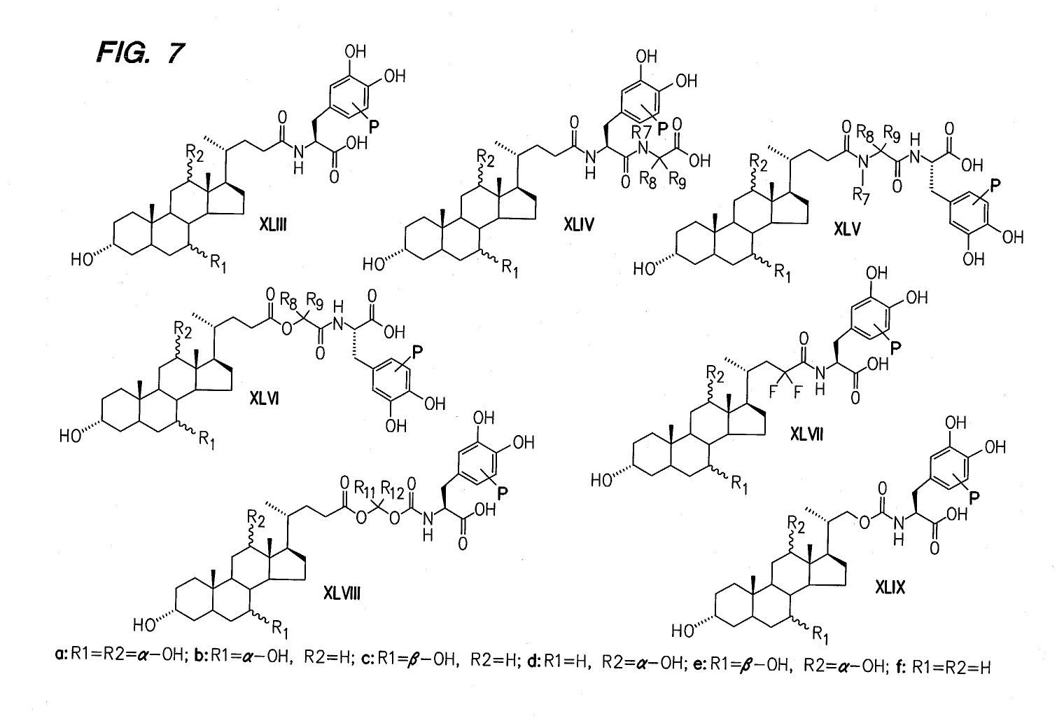

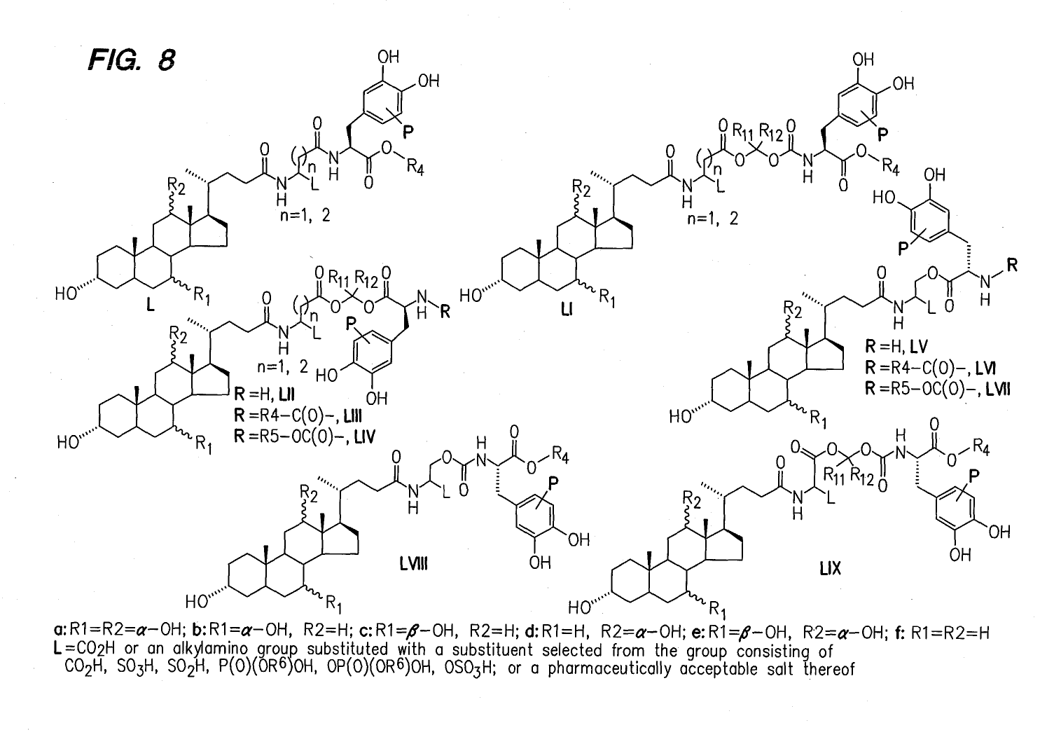

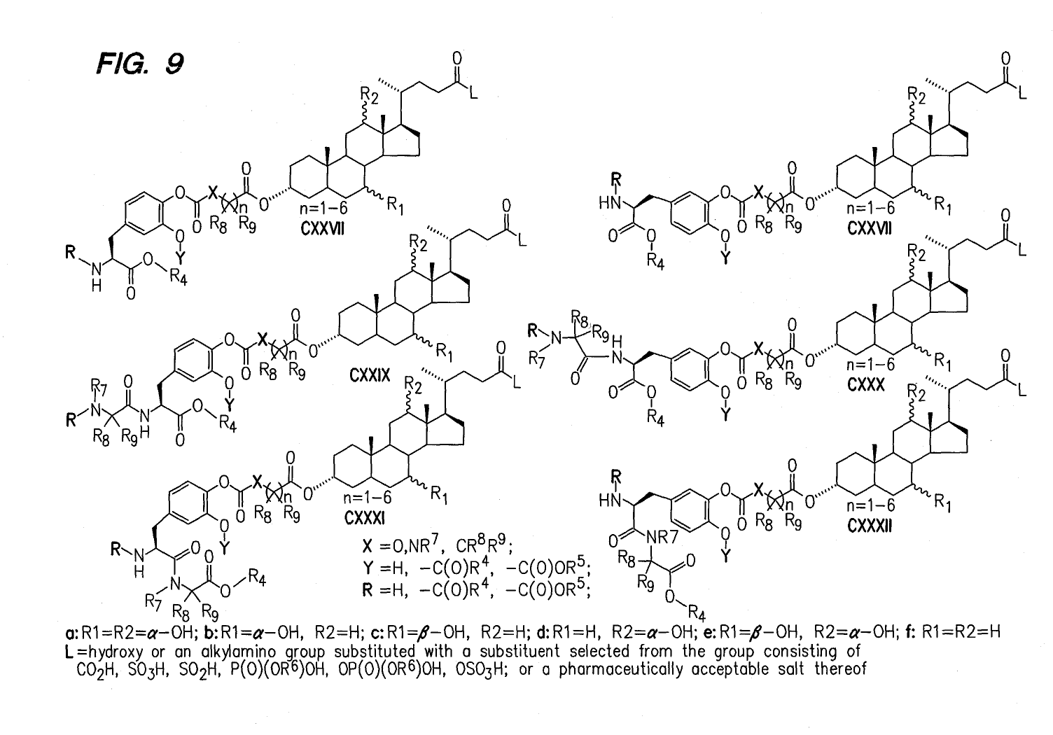

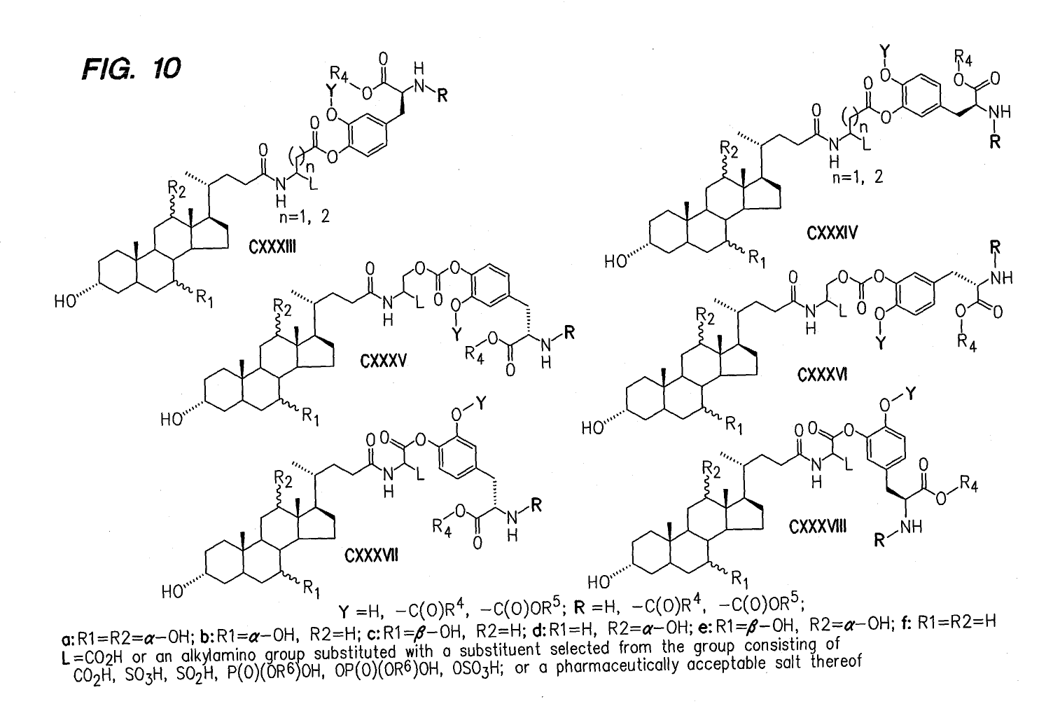

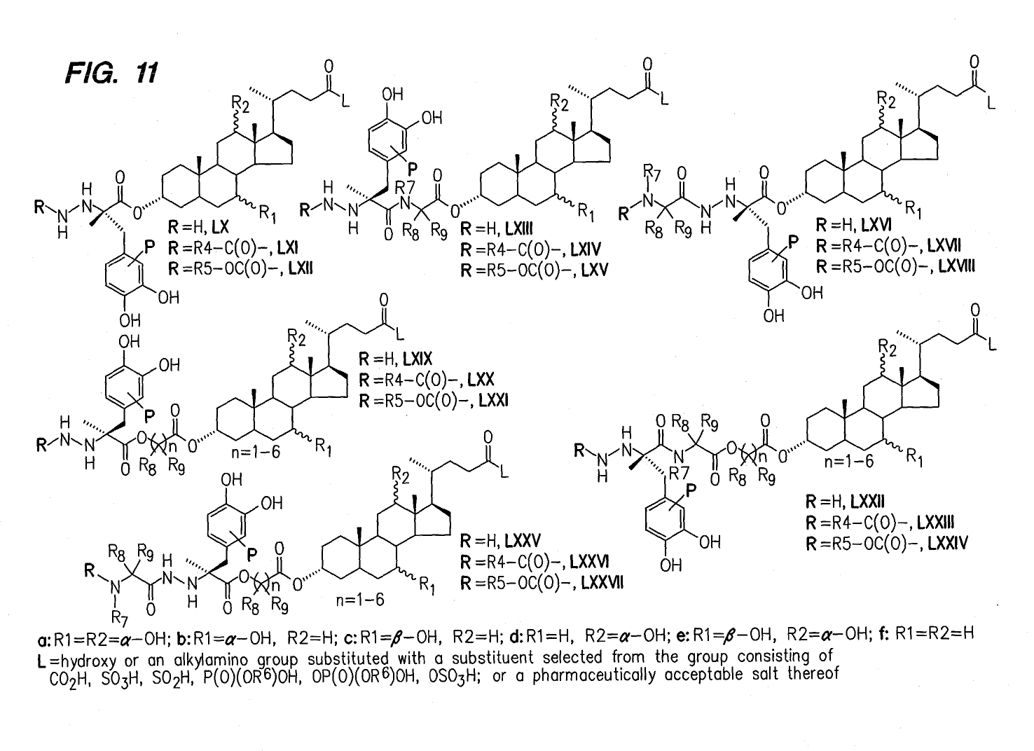

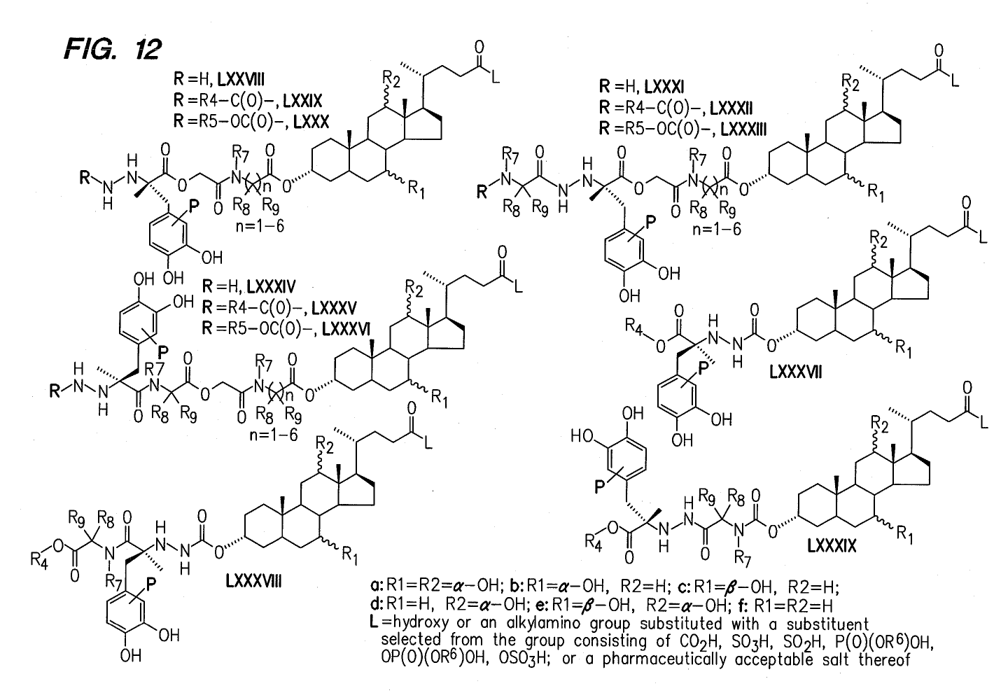

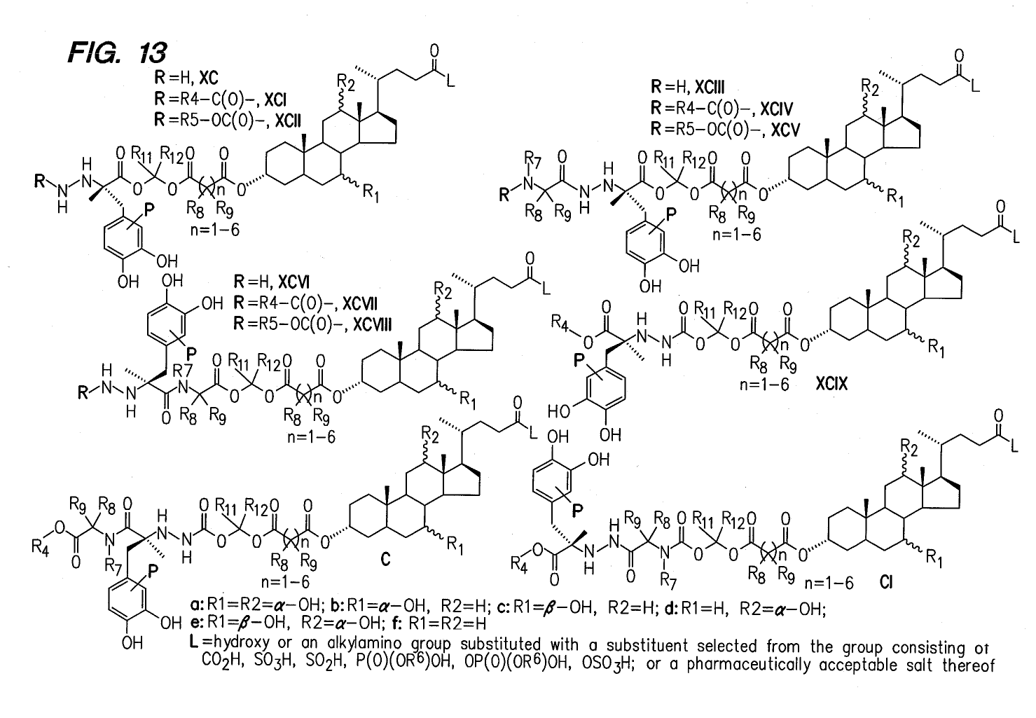

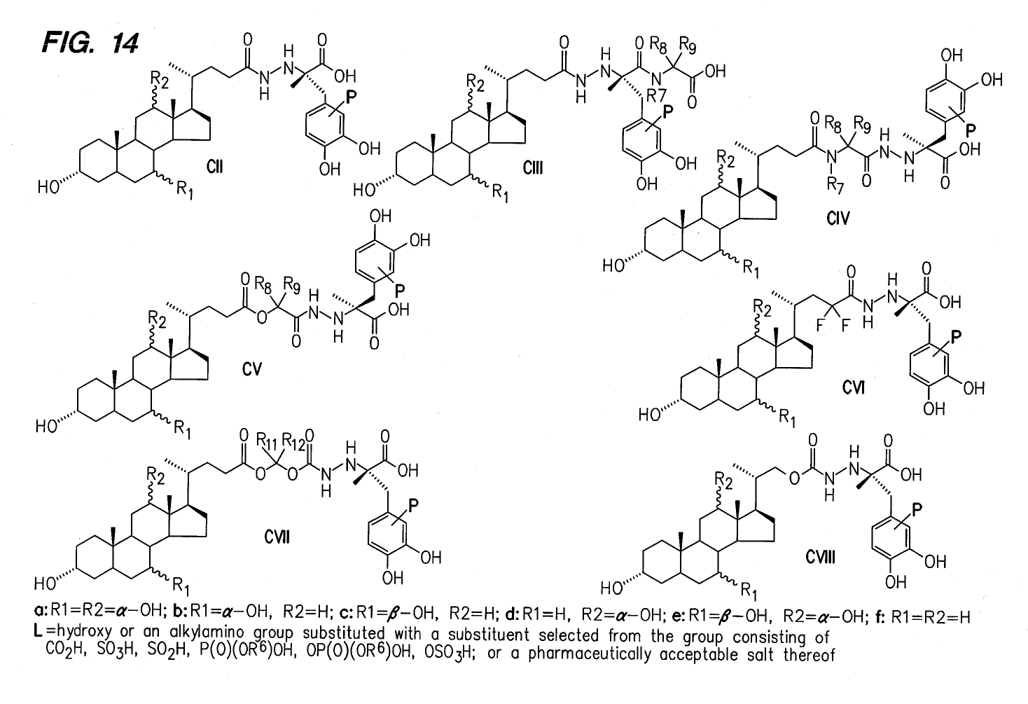

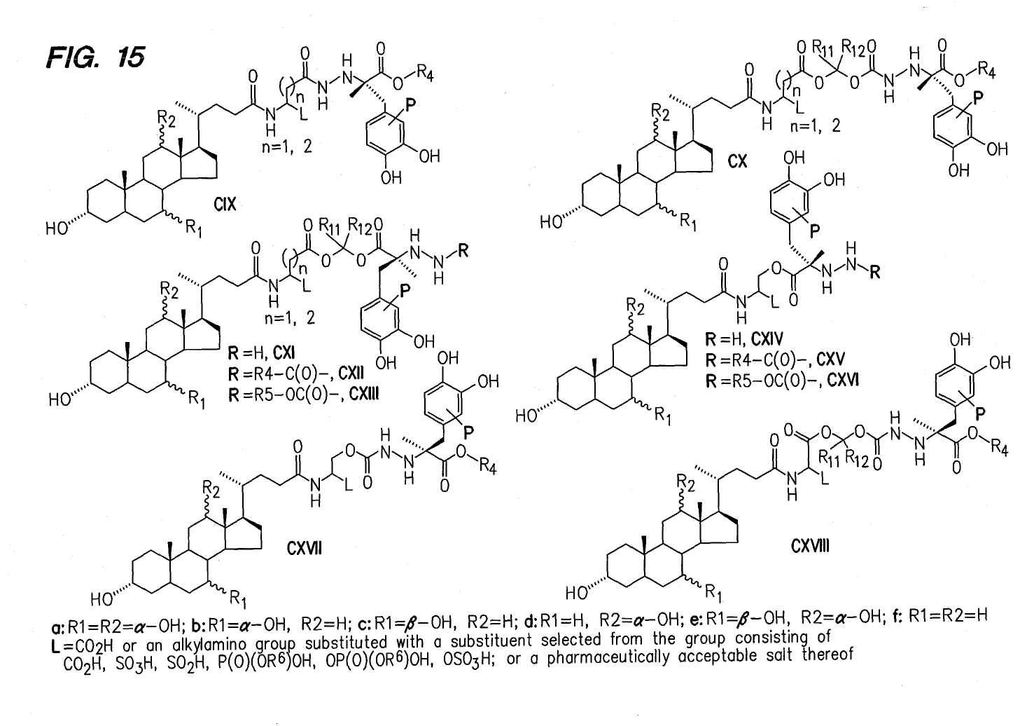

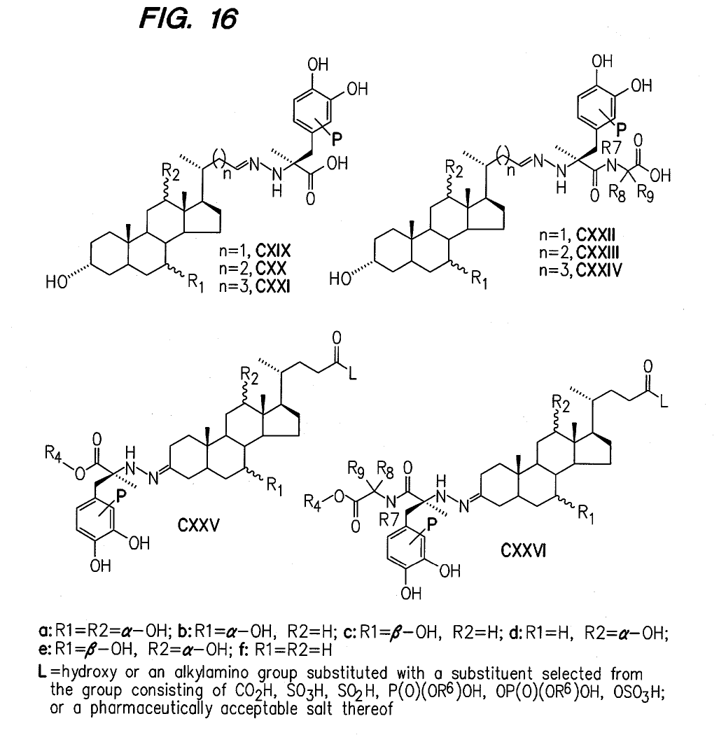

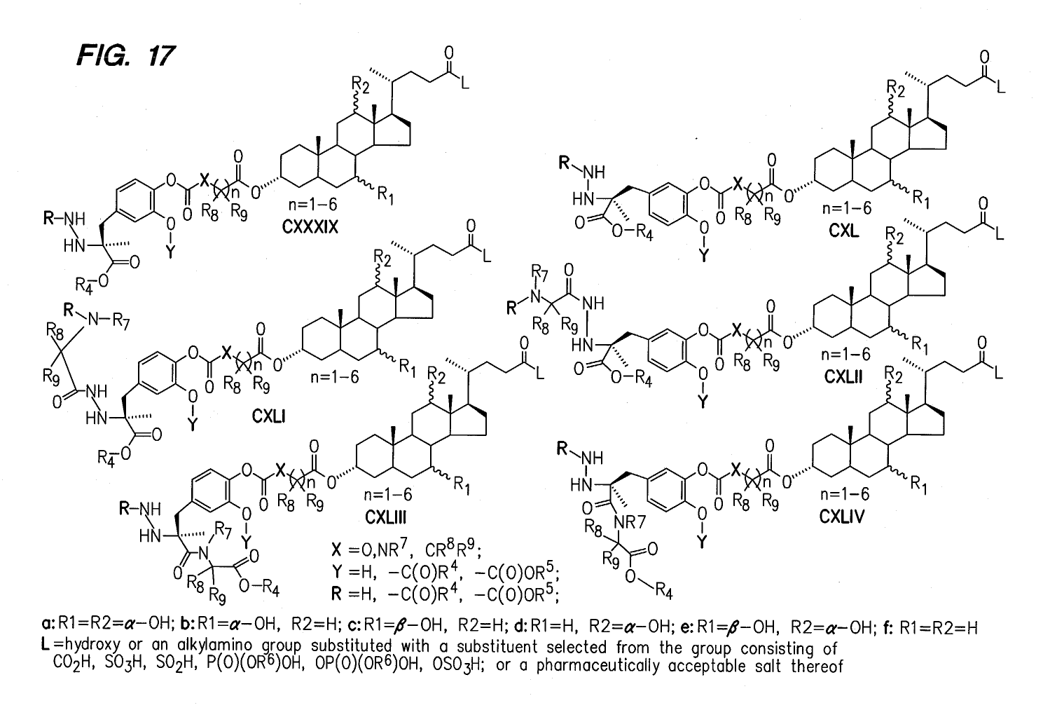

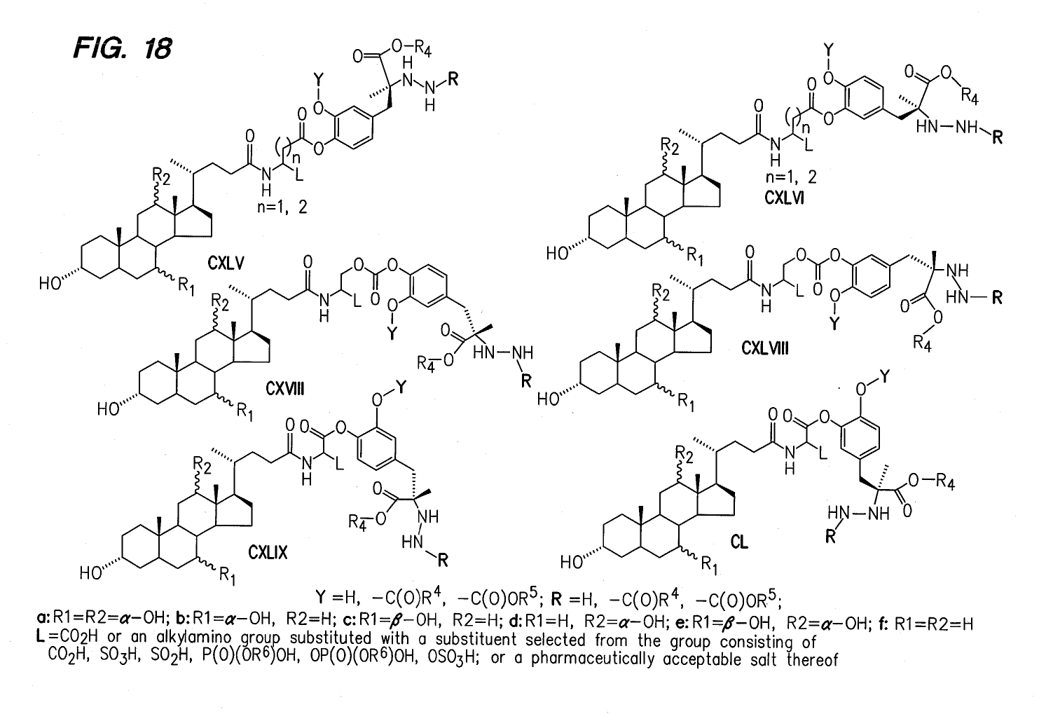

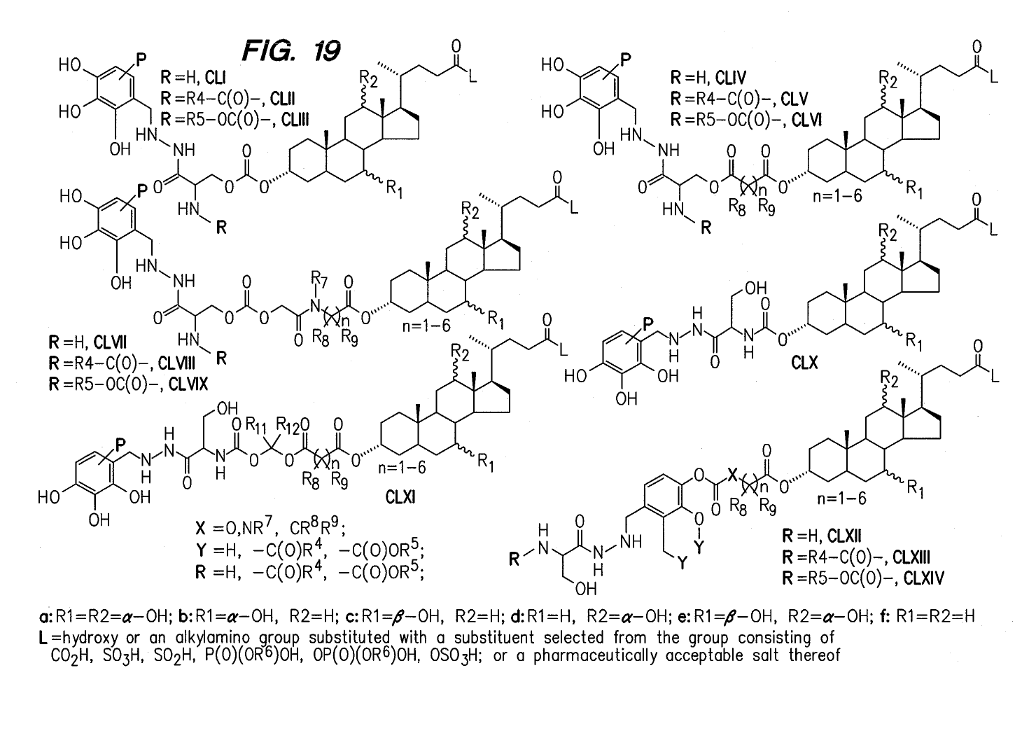

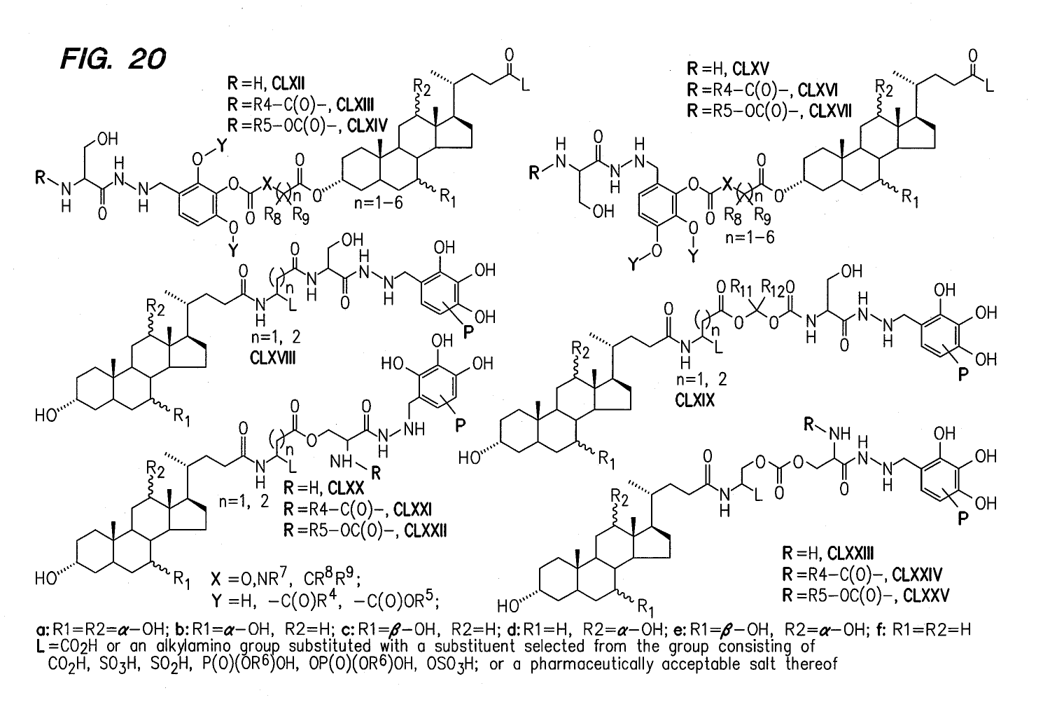

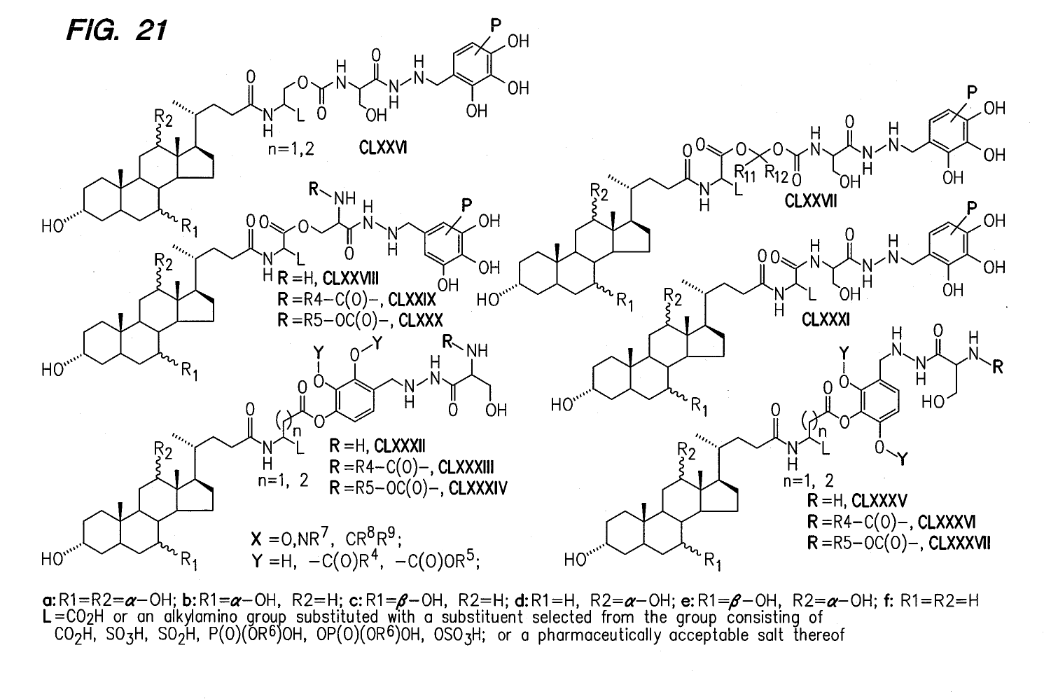

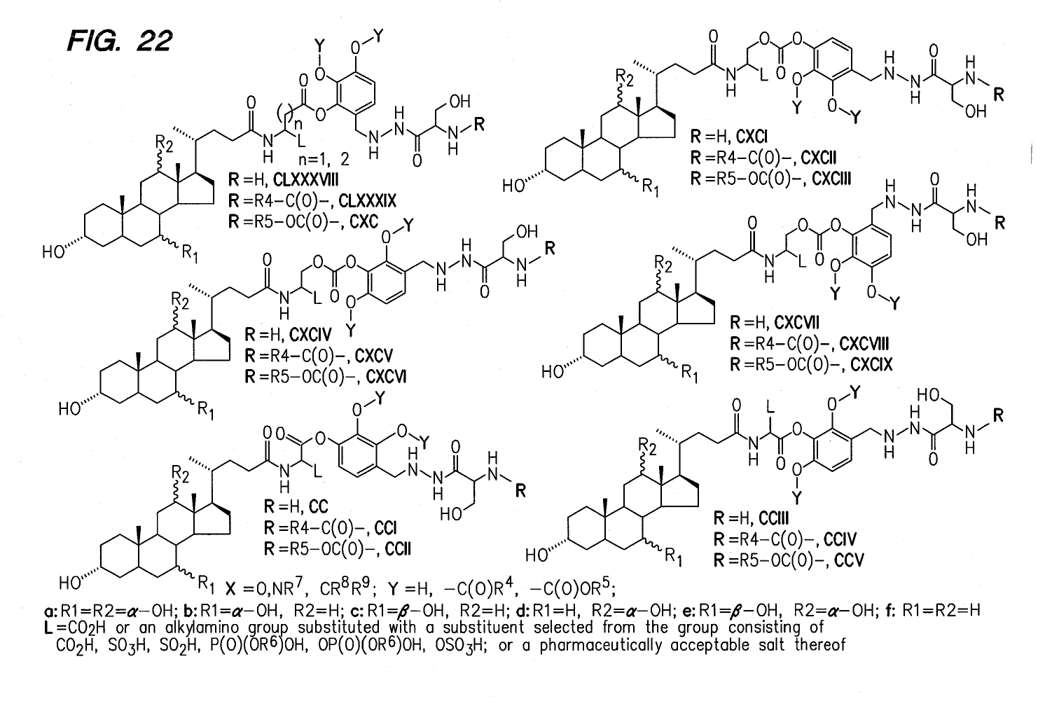

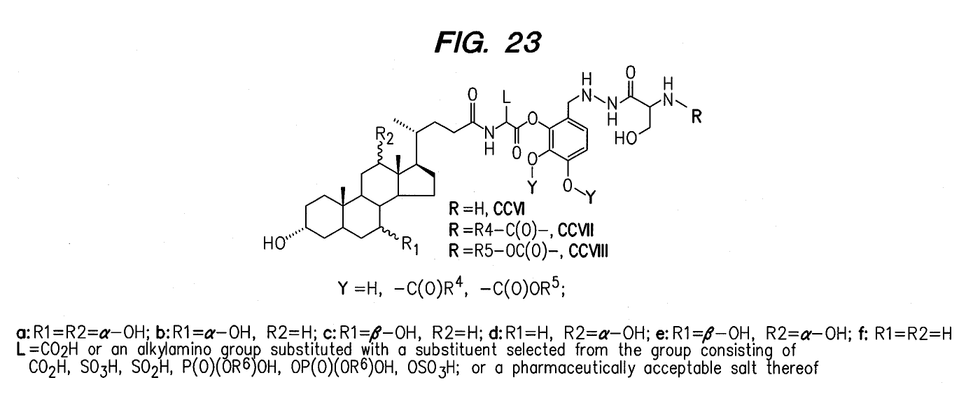

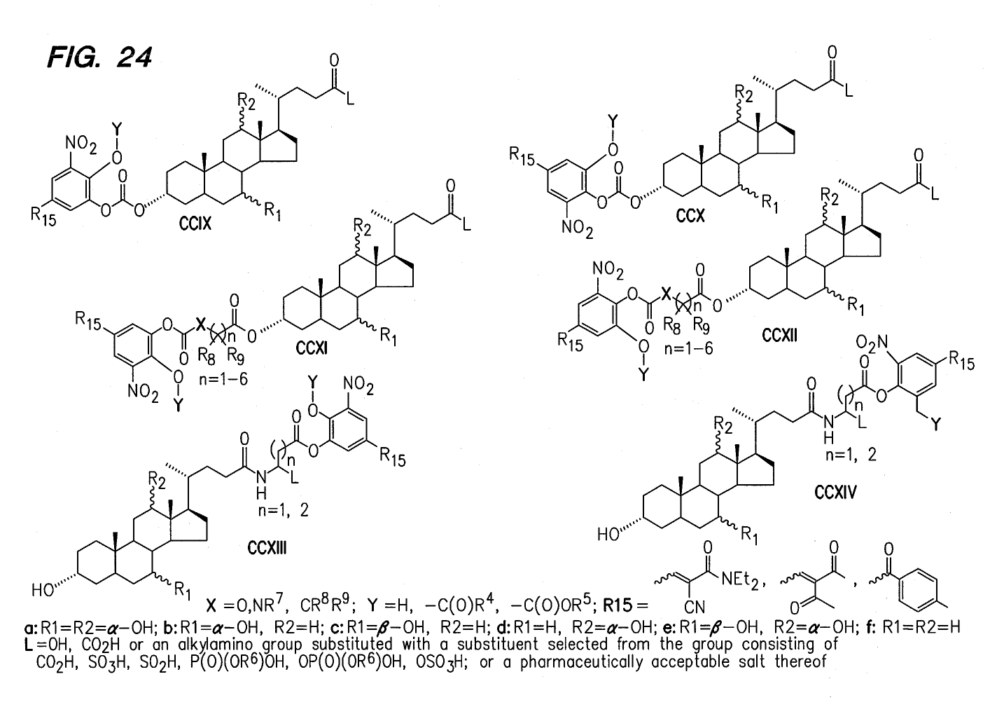

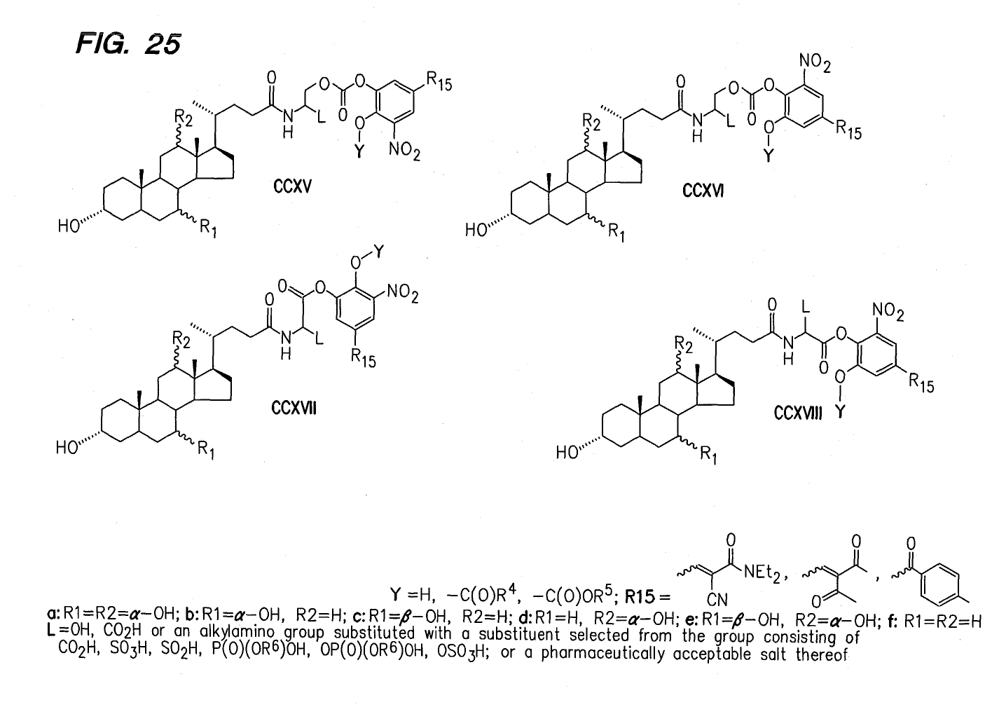

Within the scope of the present invention are bile acid prodrug derivatives that combine levodopa with one or more inhibitors of its metabolism (i.e., an AADC or COMT inhibitor). Some of these compounds are schematically represented in FIG. 3. Such multi-drug bile acid analogs undergo enterohepatic circulation and hydrolysis in vivo to provide sustained systemic blood levels of both levodopa and the AADC or COMT inhibitor. Note that co-drug compositions are disclosed in U.S. Pat. No. 6,051,576 and PCT Application WO95/20567, but active transport of such compounds by the bile acid transport system is not described therein. The present invention also includes prodrugs containing two or more units of levodopa. For example, when R⁵ in compounds IV-IX and LXIII-LXVIII is L-3,4-dihydroxybenzyl (optionally protected as described in FIG. 2) the prodrugs undergo hydrolysis in vivo to liberate 2 molecules of levodopa per molecule of prodrug. In FIG. 2, such optional protection is illustrated by the “P” depicted in the structures contained therein.

The compounds of formula (I) are also preferably the compounds having formula (I-a) or (I-b):

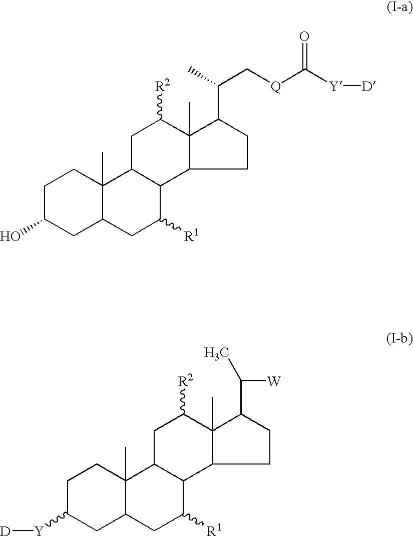

wherein

Y and Y′ are either a covalent bond or a cleavable linker group;

D and D′ are independently members selected from the group consisting of L-DOPA, a catechol O-methyl transferase inhibitor and a L-aromatic amino acid decarboxylase inhibitor;

Q is CH₂ or O;

W is a substituted alkyl group containing a moiety which is negatively charged at physiological pH, which moiety is selected from the group consisting of —COOH, —SO₃H, —SO₂H, —P(O)(OR⁶)(OH), —OP(O)(OR⁶)(OH), —OSO₃H and the like and pharmaceutically acceptable salts thereof, where R⁶ is selected from the group consisting of alkyl, substituted alkyl, aryl and substituted aryl;

R¹ and R² are one of the following combinations:

R¹ and R² are α-OH;

R¹ is α-OH and R² is H;

R¹ is β-OH and R² is H;

R¹ is H and R₂is -OH;

R¹ is β-OH and R² is α-OH; or

R¹ and R² are H;

or a pharmaceutically acceptable salt thereof.

When Y and Y′ are cleavable linker groups they are more preferably represented by the formula -X^{*}-Y^{*}-Z- where X^{*} is the linker chemistry for attachment to the drug D or D′; Y^{*} is a covalent bond or a linker moiety; and Z is the linker chemistry for attachment to the steroid.

Preferably X^{*} is selected from the group consisting of —OC(O)—, —OC(O)NR⁷—, —OC(O)OCR¹¹R¹²O—, —OC(O)OCR¹¹R¹²OC(O)—, —OC(O)OCR¹¹R¹²OC(O)O—, —OC(O)OCR¹¹R¹²OC(O)NR⁷—, —NR⁷C(O)O—, —NR⁷C(O)—, —NR⁷C(O)OCR¹¹R¹²OC(O)—, —NR⁷C(O)OCR¹¹R¹²OC(O)O—, —NR⁷CH₂NR⁷C(O)—, —C(O)O—, —C(O)S—, —C(O)NR⁷—, —C(O)NR⁷C(O)R⁷—, —C(O)OCR¹¹R¹²O—, —C(O)OCR¹¹R¹²OC(O)—, —C(O)OCR¹¹R¹²OC —C(O)OCH₂C(O)NR⁷—, —C(O)OCH₂CH₂NR⁷C(O)—, —C(O)OCH₂NR⁷C(O)—, —C(O)OCR¹¹R¹²OC(O)NR⁷—, with the underlined atom being derived from a hydroxyl, NH, carboxylic acid moiety of the drug D or D′; each R⁷ is independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl; R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R² together with the atoms to which they are attached form a cycloalkyl, substituted cycloalkyl, heterocycle or substituted heterocyclic ring.

Preferably Z is selected from the group consisting of a bond, —O—, —S—, —C(O)O—, —OC(O)O—, —NR⁷C(O)O—, —OC(O)NR⁷—, —OP(O)(OR⁶)O—, —P(O)(OR⁶)O—, —NR⁷P(O)(OR⁶)O—, —C(O)NR⁷—, —NR⁷C(O)NR⁷—, —NR⁷C(O)NR⁷—, —S(O)₂NR⁷—, —S(O)—, —S(O)₂—, —C(O)S—, —ON═, —C(O)ON═, —NR⁷C(O)ON═, —C(O)OCR¹¹R¹²ON═, and a C═C linkage, wherein R⁶—R¹² are defmed as above.

Preferably Y^{*} is a bond or a bivalent hydrocarbyl radical of 1 to 18 atoms having at least one alkylene, alkenylene or alkynylene group, with said at least one alkylene, alkenylene or alkynylene group optionally replaced with —O—, —S—, —NR⁷—, —C(O)—, —C(S)—, —OC(O)—, —C(O)O—, —SC(O)—, —C(O)S—, —SC(S)—, —C(S)S—, —C(O)NR⁷—, —NR⁷C(O)—, arylene, substituted arylene, cycloalkylene, substituted cycloalkylene, cycloalkenylene, substituted cycloalkenylene, bivalent heterocyclic group or substituted bivalent heterocyclic group.

Y^{*} is also preferably represented by the formula:

—(R^{3′})_{f}(R^{4′})_{g}(R^{5′})ₕ—

where each of R^{3′}, R^{4′} and R^{5′} are independently selected from the group consisting of alkylene, substituted alkylene, alkenylene, substituted alkenylene, alkynylene, substituted alkynylene, cycloalkylene, substituted cycloalkylene, cycloalkenylene, substituted cycloalkenylene, arylene, substituted arylene, heteroarylene, substituted heteroarylene, heterocyclene and substituted heterocyclene; and each of f, g and h are independently an integer from 0 to 3. More preferably, Y^{*} is alkylene, alkenylene or alkynylene.

Examples of Y and Y′ are members selected from the group consisting of a carbonyl group, thiocarbonyl group and radicals of formulae (vi) to (xlviii):

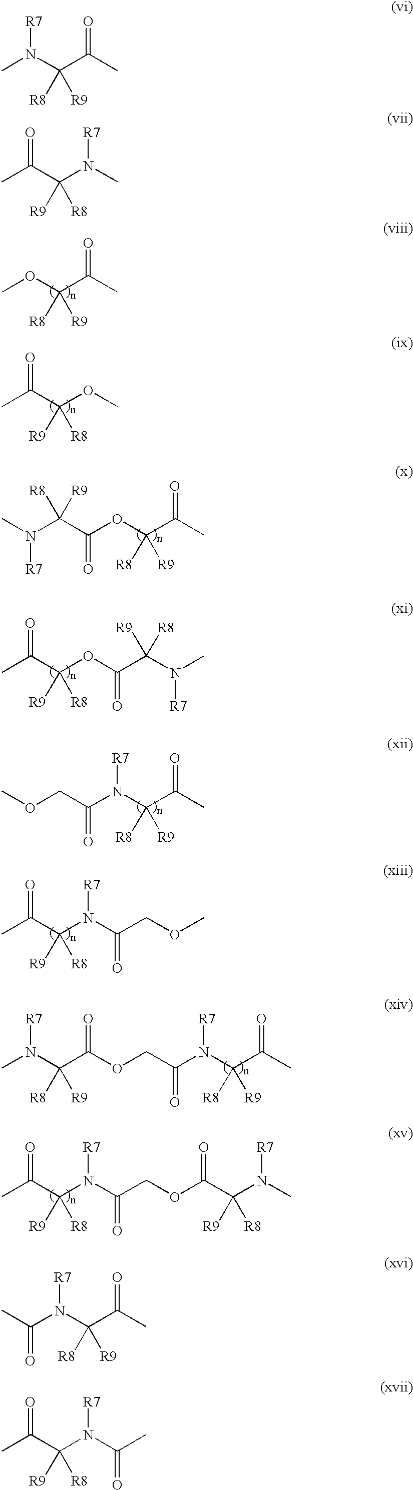

wherein:

n is an integer of 1 to 6;

each R⁷, R⁸ and R⁹ are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or, when R⁷ and R⁹ are present and attached to adjacent atoms, then R⁷ and R⁹ together with the atoms to which they are attached form a cycloalkyl, substituted cycloalkyl, heterocycle or substituted heterocyclic ring;

R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring.

### Preparation of Compounds

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis⁵ and references cited therein.

Furthermore, the compounds of this invention will typically contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Prodrugs of this invention may be prepared by methods well known in the art.^{5, 11, 12, 16, 23, 25} The disclosures of these references are herein incorporated by reference. Some of the preparative methods can be found in U.S. Provisional Application No. 60\238758.³⁰

The compounds of formula (I) above can be prepared by covalent coupling a difunctionalized linker precursor with a drug and a suitable transporter compound. The linker precursor is selected to contain at least one reactive functionality that is complementary to at least one reactive functionality on the drug and at least one reactive functionality on the transporter compound. Such complementary reactive groups are well known in the art as illustrated below:

COMPLEMENTARY BINDING CHEMISTRIES

| First Reactive Group | Second Reactive Group | Linkage |
|---|---|---|
| hydroxyl | carboxylic acid | ester |
| amine | carboxylic acid | amide |
| hydroxyl | isocyanate | urethane |
| amine | epoxide | hydroxylamine |
| sulfonyl halide | amine | sulfonamide |
| hydroxyl | alkyl/aryl halide | ether |
| aldehyde | amine/NaCNBH₄ | amine |
| ketone | amine/NaCNBH₄ | amine |
| amine | isocyanate | urea |

Suitable linker precursors include, by way of example, dicarboxylic acids, disulfonylhalides, dialdehydes, diketones, dihalides, diisocyanates,diamines, diols, mixtures of carboxylic acids, sulfonylhalides, aldehydes, ketones, halides, isocyanates, amines and diols. In each case, the linker precursor is reacted with a complementary functionality on the drug and on the transporter compound to form a compound of formula (i) above.

Examples of dicarboxylic acids useful as cleavable linkers herein include, for example, succinic acid, maleic acid, etc.

Examples of diols include, for example, polyoxyalkylene compounds of the general formula HO(alkylene-O)ₐ—H where alkylene is as defined herein and a is an integer from 1 to 20.

Examples of diamines include, for example, polyalkylene amine compounds of the general formula H₂N(alkylene-NH)ₐ—H where alkylene is as defined herein and a is an integer from 1 to 20. Reaction of the complementary functional groups to form a covalent linkage follows conventional chemical reactions. For example, drugs with a carboxylic acid group or an amine group (as described above) can be reacted under conventional conditions with an amine or a carboxylic acid to form an amide bond using conventional coupling techniques and reagents, such carbodiimides, BOP reagent and the like which are well known in the peptide art. Alternatively, amine and hydroxyl groups can be reacted with an isocyanate under conventional conditions to form a urea or carbamate linkage respectively.

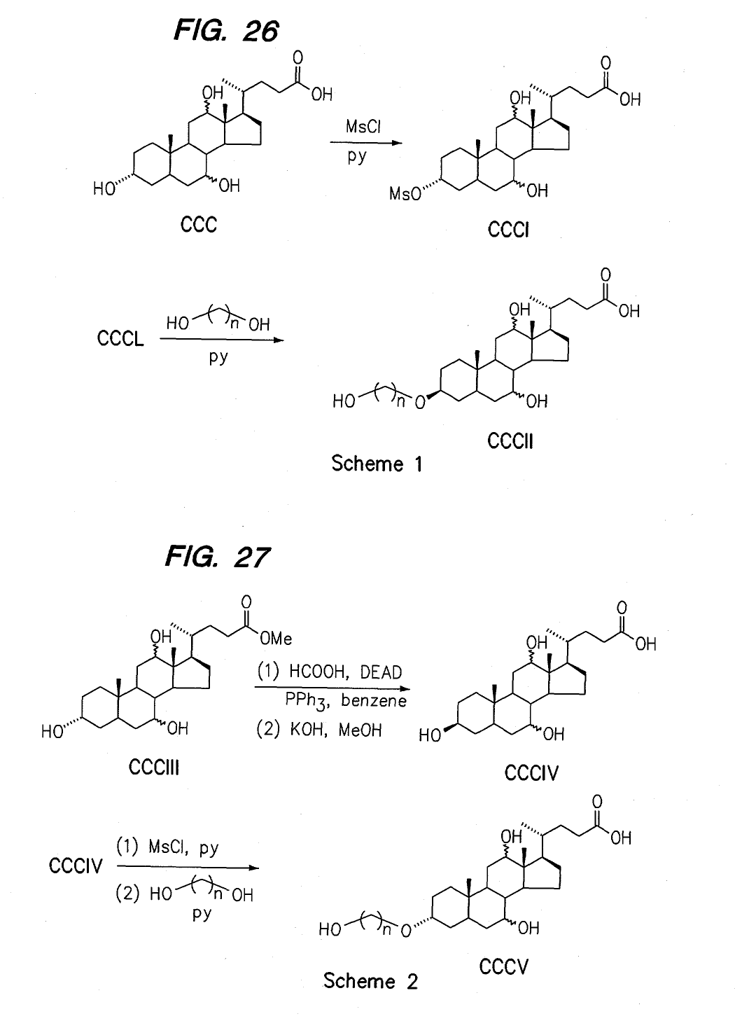

A method of preparing some bile acid intermediates having D-Y attached to position 3 of the steroid core in a β orientation, in which Y is —O(CH₂)ₙO—, with n being an integer of 1 to 17 is shown in FIG. 26. The method involves a reaction of a bile acid derivative, CCC, having a 3-α—OH group with methanesulfonyl chloride, followed by a reaction with a diol.

A method of preparing some intermediates having D-Y attached to position 3 of the steroid core in an (x orientation, in which Y is —O(CH₂)ₙO—, with n being an integer of 1 to 17 is shown in FIG. 27. The method involves a reaction of a bile acid derivative, CCCIII, having a 3-α—OH group with formic acid, DEAD, i.e. diethyl azodicarboxylate, and triphenyl phosphine, followed by a reaction with KOH in methanol to generate an intermediate, CCCIV, which is reacted with methanesulfonyl chloride and then with a diol to obtain an intermediate, CCCV.

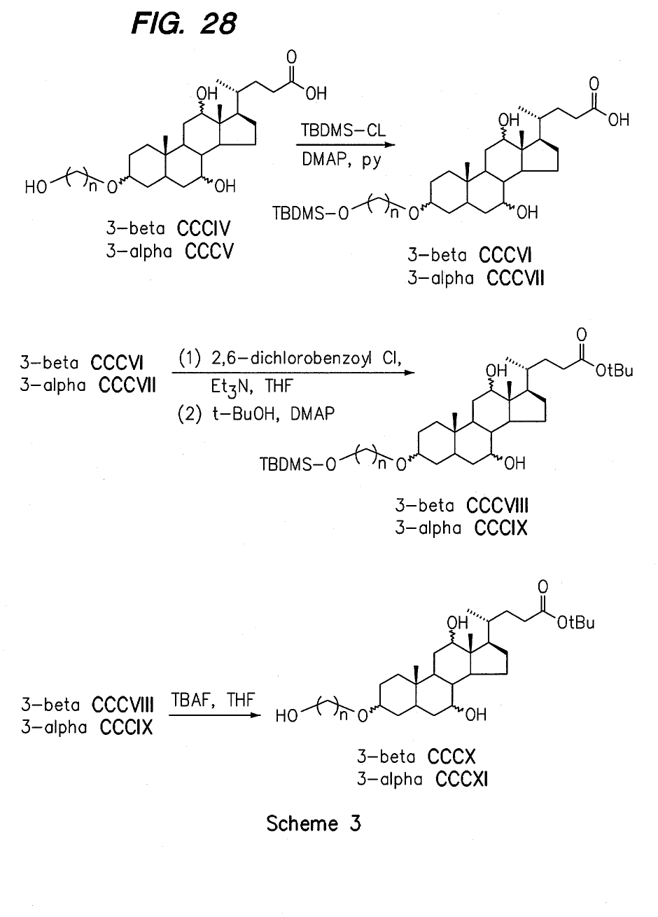

A method of preparing some bile acid intermediates, in which Y is —O(CH₂)ₙO—, with n being an integer of 1 to 17 and W is CH₂CH₂C(O)O^{t}Bu is shown in FIG. 28. The method involves first a protection of a terminal hydroxyl group attached to position 3 with TBDMS, i.e. t-butyldimethylsilyl, protection of the C-24 carboxyl group as a t-butyl ester, and then removal of TBDMS to obtain a hydroxyl intermediate, CCCX or CCCXI.

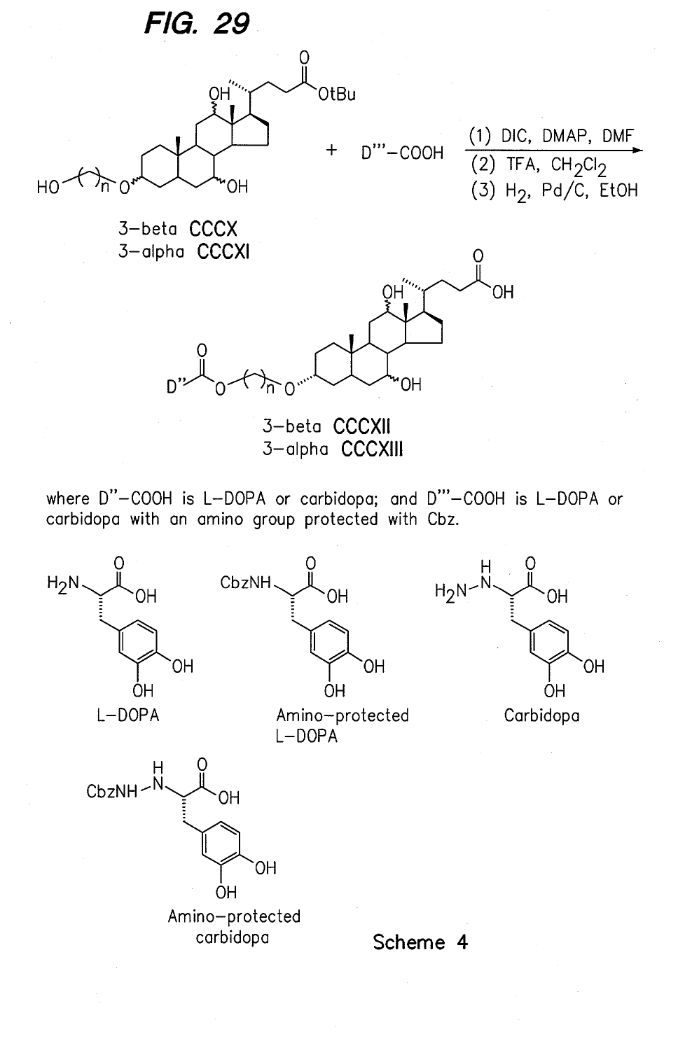

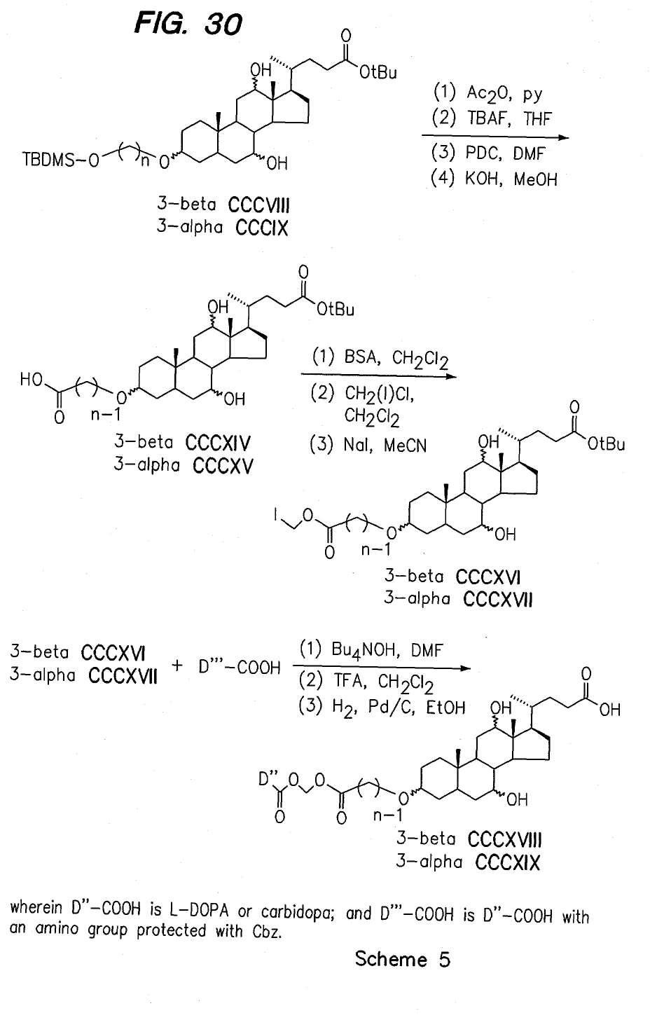

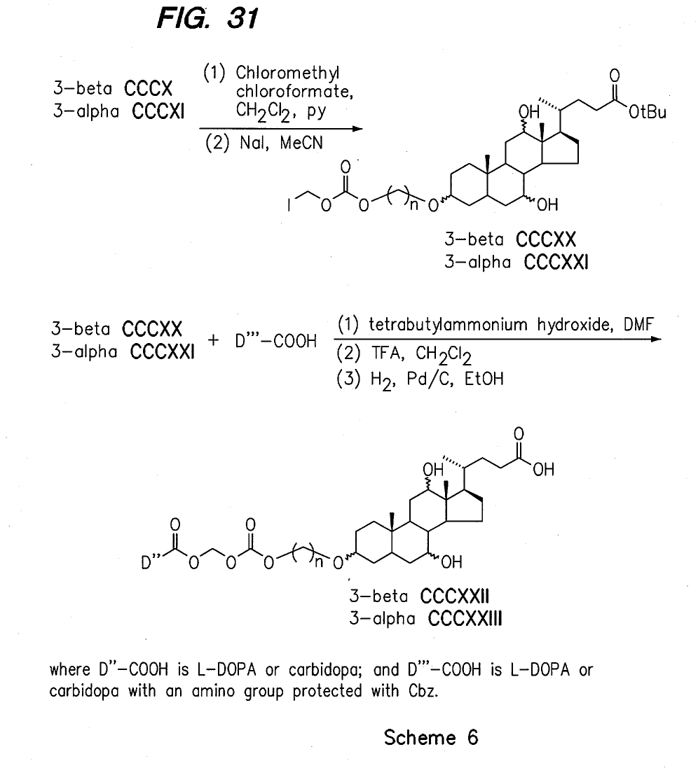

There are several methods for the preparation of some of the compounds of formula (I) where W is CH₂CH₂CO₂H, X is -Y-D, and D is L-DOPA or carbidopa by relying on the carboxyl group of L-DOPA and carbidopa to form an ester linkage with Y (see FIGS. 29-31). As shown in FIG. 29, the first method involves a reaction of the hydroxyl intermediate, CCCX or CCCXI, with L-DOPA or carbidopa with an amino group protected with Cbz, i.e. benzyloxycarbonyl, followed by the removal of the t-butyl group and then the removal of the Cbz group, to obtain compound CCCXII or CCCXIII. In the second method (FIG. 30), the intermediate, CCCVIII or CCCIX, is subjected to a series of reactions with acetic anhydride, TBAF, i.e. tetrabutylammonium fluoride, PDC, i.e. pyridinium dichromate and KOH to form an intermediate, CCCXIV or CCCXV, having a terminal carboxyl group at position 3. The intermediate, CCCXIV or CCCXV, is then converted to an iodomethyl ester, CCCXVI or CCCXVII, in a series of reactions involving chloroiodomethane and NaI. The iodomethyl ester is then reacted with the carboxyl group of an amino-protected L-DOPA or carbidopa followed by the removal of the t-butyl group and Cbz group to form a compound, CCCXVIII or CCCXIX, of formula (i) where D is linked to Y via an ester linkage. The third method (FIG. 31) is similar to the second method (FIG. 30) except that the method involves the formation of an iodomethyl carbonate intermediate, CCCXX or CCCXXI, which is obtained by a reaction of the hydroxyl intermdiate CCCX or CCCXI with chloromethyl chloroformate and NaI.

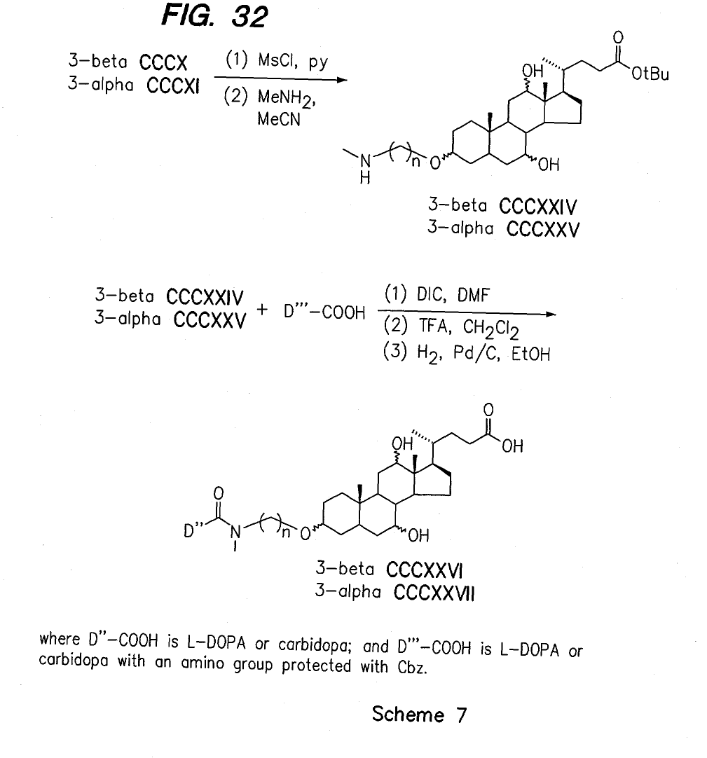

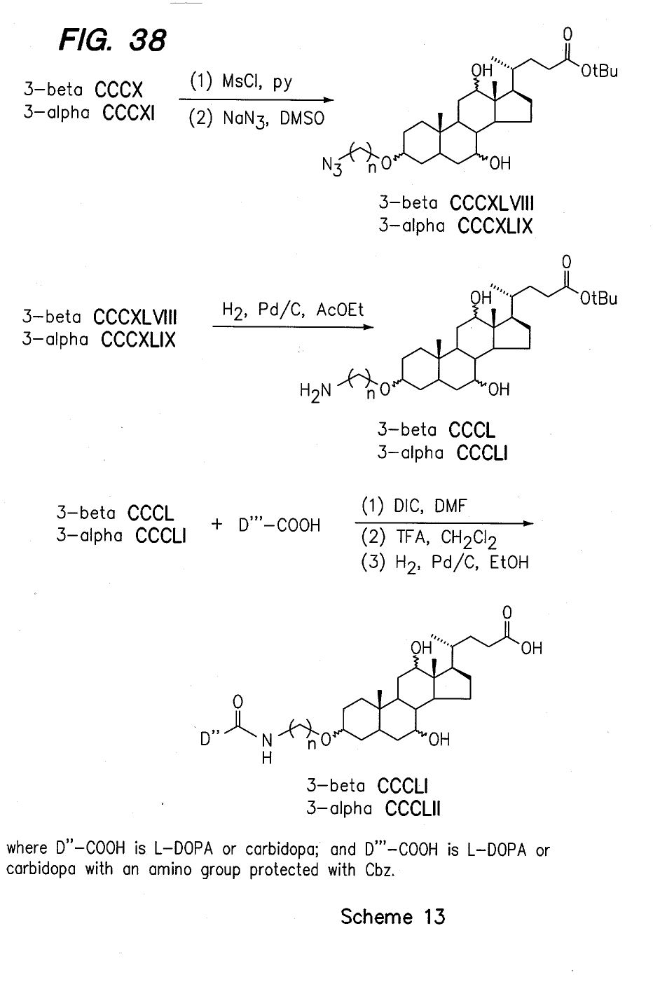

FIGS. 32 and 38 illustrate two methods of making some of the compounds of formula (I) where where W is CH₂CH₂CO₂H, X is -Y-D, and D is L-DOPA or carbidopa, with D linked to Y via an amide linkage obtained by a reaction of the carboxyl group of L-DOPA or carbidopa. The methods in FIGS. 32 and 38 both involve the formation of a mesylate intermediate by reacting the hydroxyl intermediate, CCCX or CCCXI, with methanesulfonyl chloride. In the method of FIG. 32, the mesylate intermediate is converted to an intermediate, CCCXXIV or CCCXXV, having a terminal methylamino group at position 3 via a reaction with methylamine. The intermediate, CCCXXIV or CCCXXV, is reacted with an amino-protected L-DOPA or carbidopa using DIC, i.e. diisopropyl-carbodiimide, followed by the removal of the t-butyl and Cbz protective groups to yield compounds CCCXXVI or CCCXXVII of formula (I) where D is linkted to Y via an amide linkage. The method of FIG. 38 is similar to the method of FIG. 32 except that it involves the formation of an azido intermediate, CCCXLVIII or CCCXLIX, by reacting the mesylate intermediate with sodium azide, which is converted to an amino intermediate, CCCL or CCCLI, via hydrogenation of the azido intermediate.

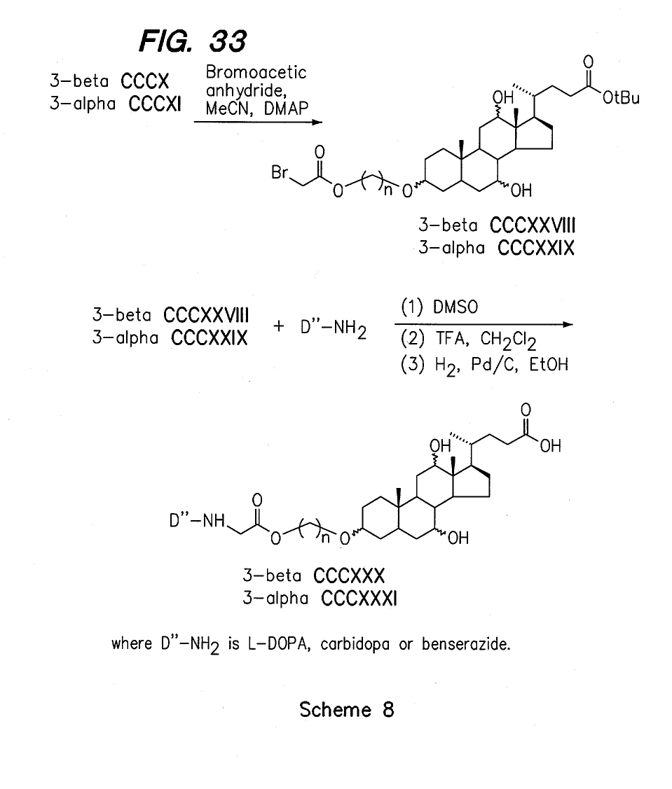

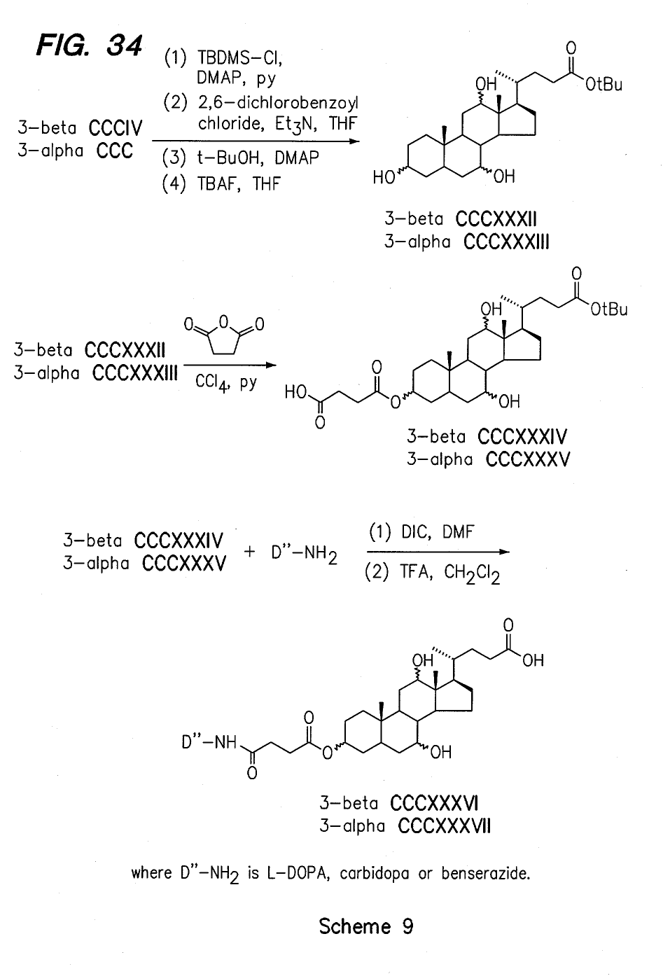

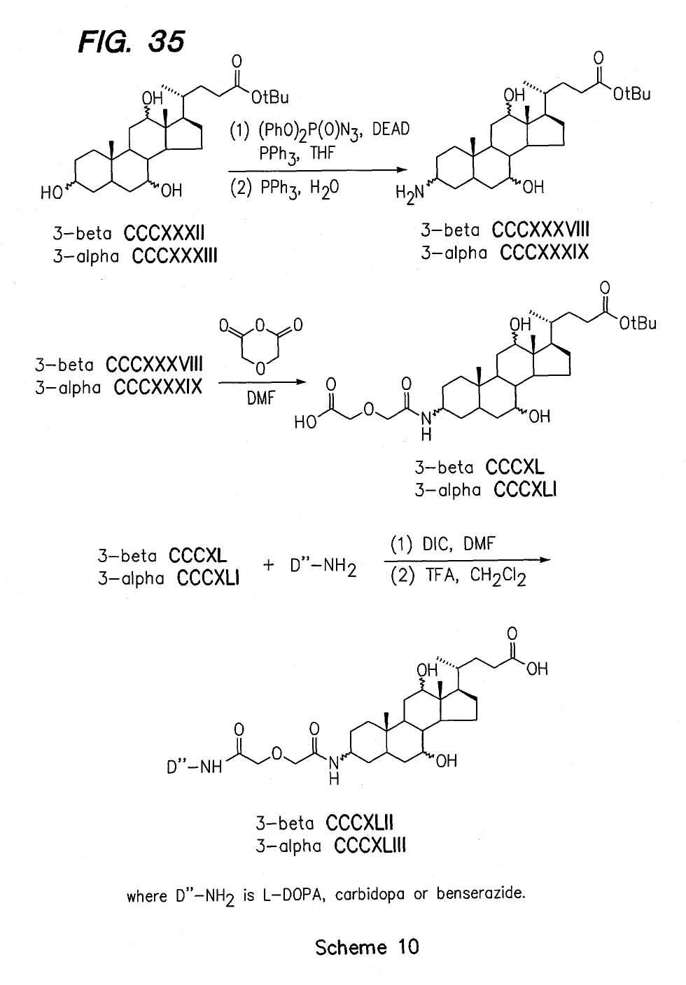

Several methods for preparing some compounds of formula (I) where W is CH₂CH₂CO₂H, X is -Y-D, and D is L-DOPA, carbidopa or benserazide, with D linked to Y via an amino group are illustrated in FIGS. 33-35. The method of FIG. 33 involves a conversion of the hydroxyl intermediate, CCCX or CCCXI, to a bromoacetate intermediate, CCCXXVIII or CCCXXIX, by bromoacetic anhydride. A nucleophilic substitution is carried out with the amino group of L-DOPA, carbidopa or benserazide as a nucleophile and the bromo group of the bromoacetic intermediate, CCCXXVIII or CCCXXIX, as a leaving group to obtain a compound, CCCXXX or CCCXXXI, of formula (I). In the method of FIG. 34, a carboxyl-protected intermediate, CCCXXXII or CCCXXXIII, is reacted with succinic anhydride to obtain an intermediate, CCCXXXIV or CCCXXXV, having a terminal carboxyl group at position 3. The carboxyl group of intermediate, CCCXXXIV or CCCXXXV, is reacted with the amino group of L-DOPA, carbidopa or benserazide using diisopropyl-carbodiimide, followed by the removal of the carboxyl protective group at position 17 to yield a compound, CCCXXXVI or CCCXXXVII, of formula (I) where D is attached to Y via an amide linkage. The method of FIG. 35 is similar to the method of FIG. 34 except that the method of FIG. 35 (1) converts the 3-hydroxyl group of intermediate CCCXXXII or CCCXXXIII, to a 3-amino group using (PhO)₂P(O)N₃ and triphenyl phosphine and (2) uses 2,6-dicarbonyl-1,4-dioxane instead of succinic anhydride to generate an intermediate having a terminal carboxyl group at position 3.

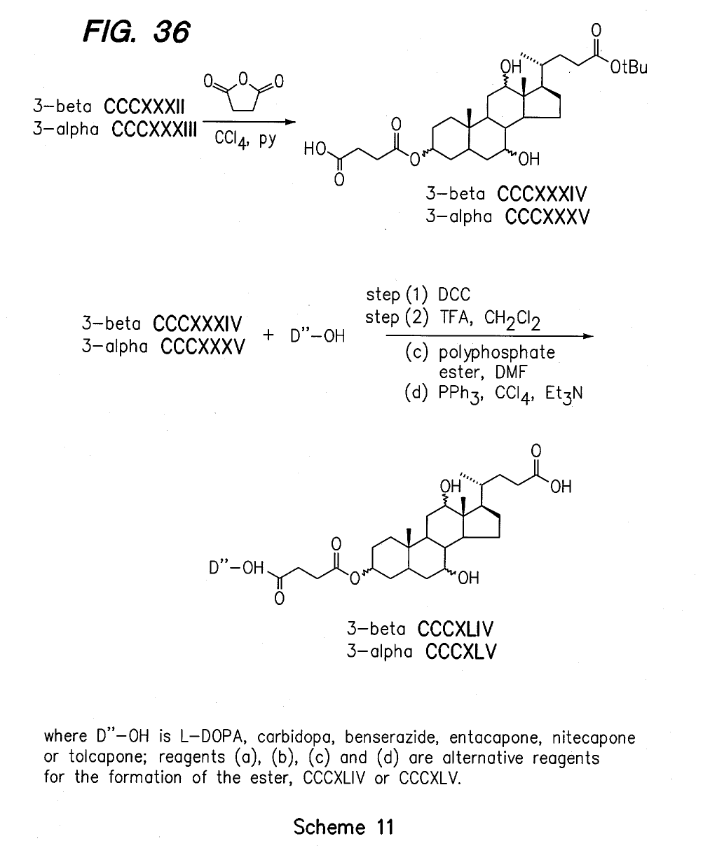

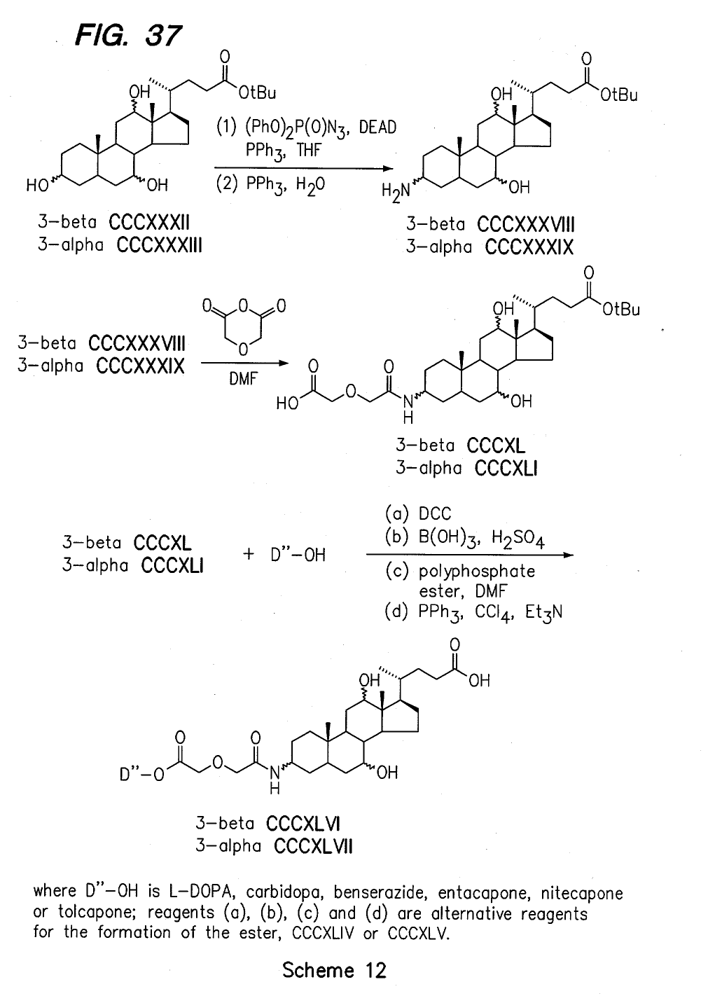

FIGS. 36 and 37 illustrate two methods for preparing some of the compounds of formula (I) where W is CH₂CH₂CO₂H, X is -Y-D, and D is L-DOPA, carbidopa, benserazide, entacapone, nitecapone or tolcapone, with D linked to Y via an ester linkage obtained by a reaction of a hydroxyl group of D with a bile acid intermediate having a terminal carboxyl group at position 3. The bile acid intermediate, CCCXXXIV, CCCXXXV, CCCXXXVIII or CCCXXXIX, having a terminal carboxyl group at position 3 is prepared from intermediate CCCXXXII or CCCXXXIII using succinic anhydride in the method of FIG. 36 or 2,6-dicarbonyl-1,4-dioxane in the method of FIG. 37. The terminal carboxyl group at position 3 of the bile acid intermediate, CCCXXXIV, CCCXXXV, CCCXL or CCCXLI, is reacted with a hydroxyl group of L-DOPA, carbidopa, benserazide, entacapone, nitecapone or tolcapone using DCC, i.e. dicyclohexylcarbodiimide, to form a compound, CCCXLIV, CCCXLV, CCCXLVI or CCCXLVII, of formula (I) where D is linked to Y via an ester linkage.

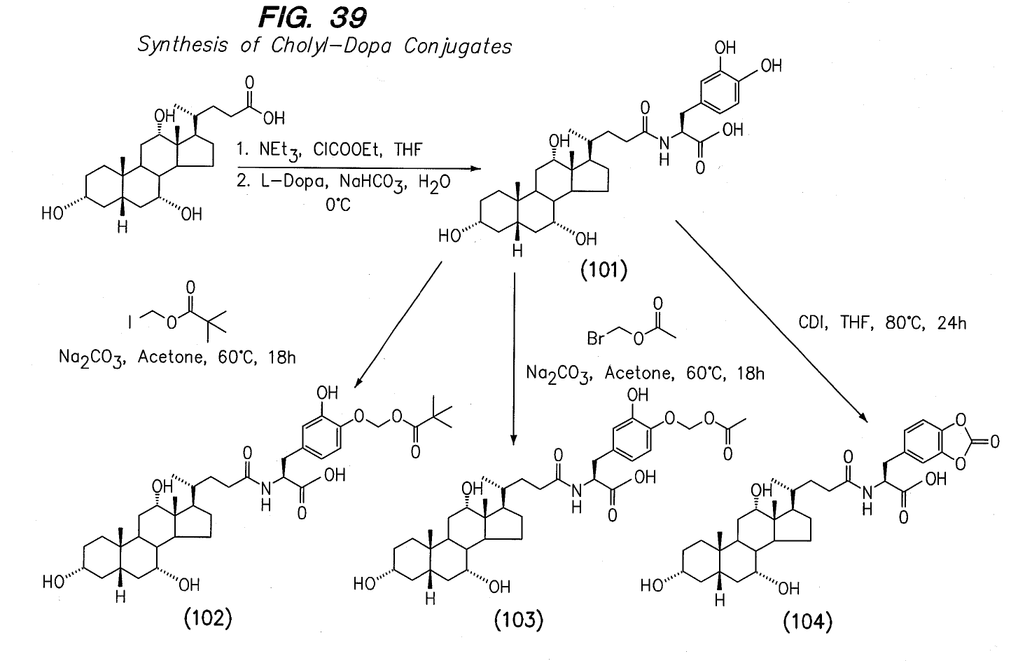

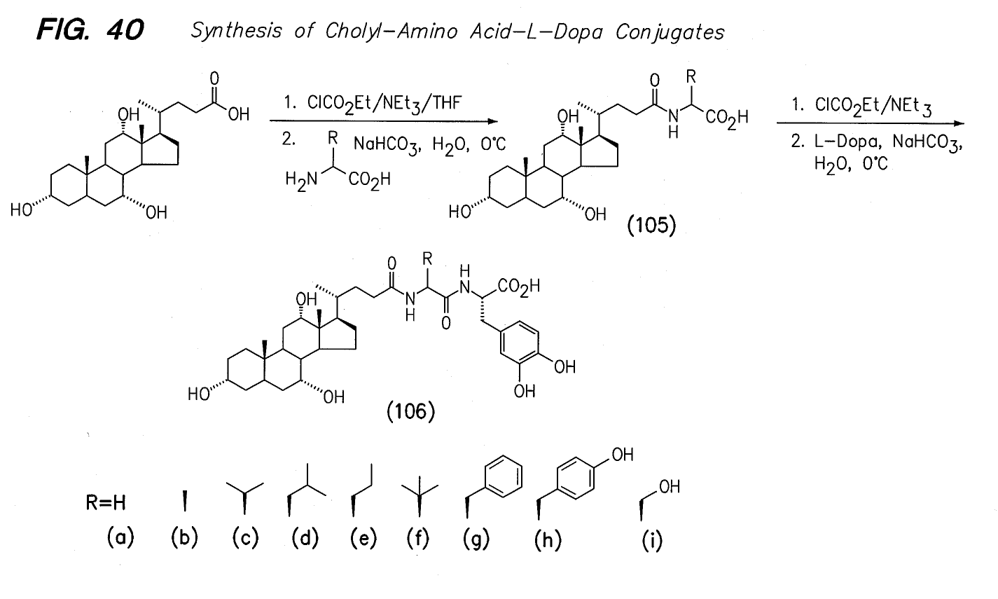

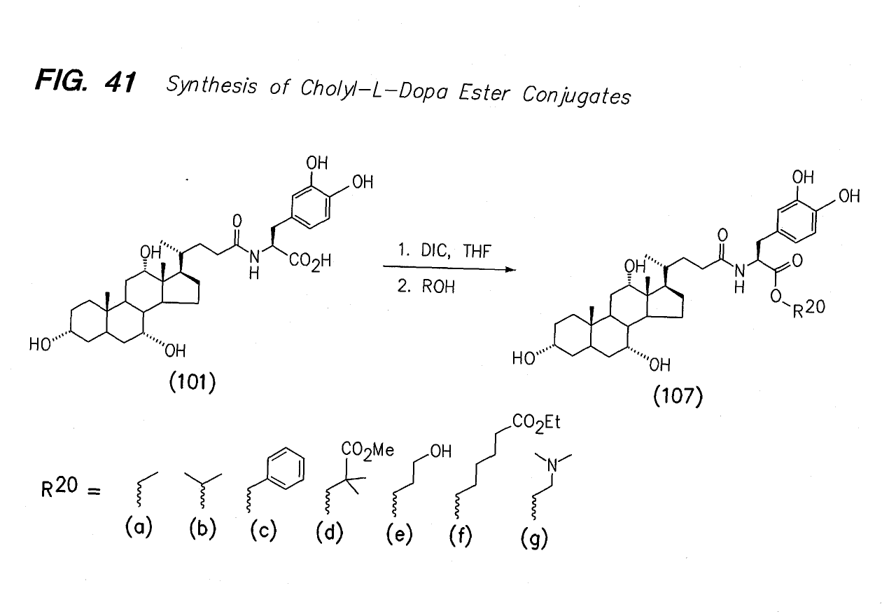

FIGS. 26-38 illustrate the preparation of some of the compounds of formula (I) where X is -Y-D. Compounds of formula (I) wherein W is -M-Y′-D′ can be prepared using methods similar to the methods of FIGS. 26-38 by applying similar reactions to a substituent at position 17, instead of position 3, of the steroid core of the bile acid intermediate. Such modifications of the methods of FIGS. 27-38 are within the knowledge of one skilled in the art and are exemplified in FIGS. 39-41.

### Utility

The compounds of this invention are useful in treating Parkinsonism by administration of one or more of the compounds of formula (I), preferably by the oral route, to a mammalian subject in need of the treatment. In a human subject weighing 70 kg, a compound of formula (I) can be administered at a dose of about 10 mg to about 10 g a day, preferably about 100 mg to about 1 g a day. The dose can be adjusted by one skilled in the art based on factors, e.g. the body weight and/or condition of the subject treated, the severity of the Parkinson's disease, and the incidence of side effects known in the art. Another aspect of the present invention relates to the use of the compound of formula (I) in the preparation of a pharmaceutical for the treatment of Parkinsonism.

### Pharmaceutical Formulations

When employed as pharmaceuticals, the compounds of this invention are usually administered in the form of pharmaceutical compositions that are administered by oral routes. Such compositions are prepared in a manner well known in the pharmaceutical art and comprise at least one active compound.

This invention also includes pharmaceutical compositions that contain, as the active ingredient, one or more of the compounds of this invention associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, etc. containing, for example, up to 90% by weight of the active compound using, for example, soft and hard gelatin capsules.

In preparing a formulation, it may be necessary to mill the active compound to provide the appropriate particle size prior to combining with other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. ˜40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in unit dosage form, each dosage containing about 1 mg to about 6 g of the active ingredient. “Unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 mg to about 2 g of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

The following synthetic and biological examples are offered to illustrate this invention and are not to be construed in any way as limiting the scope of this invention. Unless otherwise stated, all temperatures are in degrees Celsius.

### EXAMPLES

In the examples below, the following abbreviations have the following meanings. If an abbreviation is not defined, it has its generally accepted meaning.

| | |
|---|---|
| ATCC = | American Type Tissue Culture |
| CHO = | Chinese hampster ovary |
| CPM = | counts per minute |
| DMEM = | Dulbecco's minimum eagle medium |
| EDTA = | ethylene diamine tetraacetic acid |
| GDC = | glycodeoxycholate |
| GTP = | guanosine 5′-triphosphate |
| h = | hour |
| Hz = | hertz |
| IBAT = | intestinal bile acid transporter |
| kg = | kilogram |
| LBAT = | liver bile acid transporter |
| LCMS = | liquid chromatography/mass spectroscopy |
| M = | molar |
| mg = | milligram |
| mL = | milliLiter |
| mmol = | millimol |
| mm = | millimeter |
| mM = | millimolar |
| min. = | minute |
| MRM = | multiple reaction monitoring |
| MS = | mass spectroscopy |
| mV = | millivolts |
| mΩ = | milliohms |
| PBS = | phosphate buffered saline |
| PEG400 = | polyethylene glycol 400 |
| Penstrep = | penicillin/streptomycin |
| THF = | tetrahydrofuran |
| TLC = | thin layer chromatography |
| μA = | microamperes |
| μg = | microgram |
| μL = | microliter |
| μM = | micromolar |
| μm = | micron |

### EXPERIMENTAL METHODS

The synthesis of conjugates of bile acid / L-DOPA and L-DOPA derivatives, as recited in the examples below, is illustrated in FIG. 39-41, attached.

### EXAMPLE 1

### Synthesis of Cholyl-DOPA (101)

To an ice-cold solution containing cholic acid (816 mg, 2 mmol) and triethylamine (0.556 mL, 4 mmol) in anhydrous THF (100 mL) was added ethyl chloroformate (0.211 mL, 2.2 mmol). The reaction mixture was stirred at 0° C. for 30 min. A solution of L-DOPA (788 mg, 4 mmol) and NaHCO₃ (420 mg, 5 mmol) in water (10 mL) was added at 0C., then stirred for 30 min. at 0° C., and for a further 30 min. at room temperature. After removal of THF in vacuo, aqueous citric acid (20 mL) was added. The product was extracted with ethyl acetate (3×30 ML) and the combined organic phase was dried over MgSO₄ and concentrated to dryness. Chromatography on a silica gel column eluting with 5% MeOH/EtOAc gave the desired Cholyl-DOPA product (101) (880 mg, 75%). MS (ESI) m/z 588.33 (M+H⁺). ¹H NMR (CD₃OD, 400 MHz, characteristic resonances only): 6.64 (d, 1H, J=8Hz), 6.64 (d, 1H, J=2Hz), 6.52 (dd, 1H, J=2Hz, J=8Hz), 4.56 (m, 1H), 3.06-2.75 (m, 2H), 0.98 (d, 3H, J=6.4Hz), 0.91 (s, 3H), 0.68 (s, 3H).

### EXAMPLE 2

### Synthesis of Cholyl-Dopa-(3,4-carbonate) (104)

Cholyl-DOPA (59 mg, 0.1 mmol) was dissolved in anhydrous THF (30 mL), 1, 1′-carbonyldiimidazole (32 mg, 0.2 mmol) was added and the mixture heated under reflux for 24 h. The reaction was monitored to completion by TLC (10% MeOH/EtOAc). After removal of the solvent in vacuo, the residue was dissolved in EtOAc, and washed with aqueous citric acid. The organic phase was dried over MgSO₄ and concentrated to dryness. Chromatography on a silica gel column eluting with 5 % MeOH/EtOAc gave the desired cyclic carbonate product (104) (15 mg, 24%). MS (ESI) m/z 614.38 (M+H⁺). ¹H NMR (CD₃OD, 400 MHz, characteristic resonances only): 7.25 (m, 2H), 7.17 (m,1H), 4.09 (m, 1H), 2.92-2.77 (m, 2H), 0.98 (d, 3H, J=6.4Hz), 0.90 (s, 3H), 0.69 (s, 3H).

### EXAMPLE 3

### Synthesis of Cholyl-DOPA-(4-pivaloyloxymethyl) (102)

Cholyl-DOPA (400 mg, 0.68 mmol) was dissolved in anhydrous acetone (20 mL), sodium carbonate (144 mg, 1.4 mmol) was added and the mixture stirred at room temperature for 15 min. In a separate flask, sodium iodide (300 mg, 2 mmol) was dissolved in anhydrous acetone (10 mL) and chloromethylpivalate (144 μL, 1 mmol) was added at once. After stirring at room temperature for 30 min, the in situ-generated iodomethylpivalate was transferred to the flask containing Cholyl-DOPA and sodium carbonate. The mixture was heated in an oil bath at 70° C. for 18 h. The reaction was monitored to completion by TLC (10%MeOH/EtOAc). After removal of the solvent in vacuo, the residue was dissolved in EtOAc and washed with aqueous citric acid and 0.1% Na₂S₂O₃. The organic phase was dried over MgSO₄ and concentrated to dryness. Chromatography on a silica gel column eluting with 2% MeOH/EtOAc gave the desired product Cholyl-DOPA-(4-pivaloyloxymethyl) (102) (210 mg, 44%). MS (ESI) m/z 702.44 (M+H⁺). ¹H NMR (CD₃₀D, 400 MHz, characteristic resonances only): 6.66 (d, 1H, J=8 Hz), 6.64 (d, 1H, J=2 Hz), 6.52 (dd, 1H, J=2 Hz, J=8 Hz), 5.55 (dd, 2H, J=2.8 Hz, J=17 Hz), 4.58 (m,1H), 3.01-2.75 (m, 2H), 1.19 (s, 9H), 0.98 (d, 3H, J=6.4 Hz), 0.91 (s, 3H), 0.68 (s, 3H).

### EXAMPLE 4

### Synthesis of Cholyl-DOPA-(4-acetoxymethyl) (103)

Cholyl-DOPA (587 mg, 1 mmol) was dissolved in anhydrous acetone (30 mL), sodium carbonate (144 mg, 1.4 mmol) was added and the mixture stirred at room temperature for 15 min. Bromomethylacetate (155 μL, 1.5 mmol) was added and the mixture heated in an oil bath at 70° C. for 18 h. The reaction was monitored to completion by TLC (10% MeOH/EtOAc). After removal of the solvent in vacuo, the residue was dissolved in EtOAc and washed with aqueous citric acid. The organic phase was dried over MgSO₄ and concentrated to dryness. Chromatography on a silica gel column eluting with 2% MeOH/EtOAc gave the desired product Cholyl-DOPA-(4-acetoxymethyl) (103) (240 mg, 36%). MS (ESI) m/z 660.22 (M+H⁺). ¹H NMR (CD₃D, 400 MHz, characteristic resonances only): 6.66 (d, 1H, J=8 Hz), 6.63 (d, 1H, J=2 Hz), 6.51 (dd, 1H, J=2 Hz, J=8 Hz), 5.72 (dd, 2H, J=2.8 Hz, J=15.2 Hz), 4.56 (m, 1H), 3.02-2.75 (m, 2H), 2.06 (s, 3H), 0.98 (d, 3H, J=6.4 Hz), 0.90 (s, 3H), 0.68 (s, 3H).

### EXAMPLE 5

### In Vitro Compound Transport Assays with IBAT and LBAT-Expressing Cell Lines

### (a) Inhibition of Radiolabeled Taurocholate Uptake

CHO cells transfected with the IBAT or LBAT transporter were seeded into 96-well microtiter plates at 100,000 cells/well in 100 μL DMEM containing 10% serum, glutamine and Penstrep. After overnight incubation the media was removed and test compound (25 μL) added at 2× the final desired concentration. Tritiated taurocholate (50,000 CPM/well) was diluted with cold substrate to a final concentration of 5 μM and 25 μL/well of this mixture was added to the plate. After incubating for 1 h at room temperature the solution was removed and the plate washed 4× with PBS at 4° C. 200 μL/well of scintillant is added and the plate then read in a Wallac microbeta counter. The inhibition data is processed by standard methods to calculate an inhibition constant Kᵢ for the test compound.

### (b) Analysis of Electrogenic Transport in Xenopus Oocytes RNA preparation:

Human IBAT Transporter cDNAs were subcloned into a modified pGEM plasmid that contains 5′ and 3′ untranslated sequences from the Xenopus β-actin gene. These sequences increase RNA stability and protein expression. Plasmid cDNA was linearized and used as template for in vitro transcription (Epicentre Technologies transcription kit, 4:1 methylated:non-methylated GTP).

Xenopus oocyte isolation. Xenopus laevis frogs were anesthetized by immersion in Tricaine (1.5 g/mL in deionized water) for 15 min. Oocytes were removed and digested in frog ringer solution (90 mM NaCl, 2 mM KCl, 1 mM MgCl₂, 10 mM NaHEPES, pH 7.45, no CaCl₂) with 1 mg/mL collagenase (Worthington Type 3) for 80-100 min with shaking. The oocytes were washed 6 times, and the buffer changed to frog ringer solution containing CaCl₂(1.8 mM). Remaining follicle cells were removed if necessary. Cells were incubated at 16° C., and each oocyte injected with 10-20 μg RNA in 45 μL solution.

Electrophysiology measurements. Transport currents were measured 2-14 days after injection, using a standard two-electrode electrophysiology set-up (Geneclamp 500 amplifier, Digidata 1320/PCLAMP software and ADInstruments hardware and software were used for signal acquisition). Electrodes (₂-4 mΩ) were microfabricated using a Sutter Instrument puller and filled with 3M KCl. The bath was directly grounded (transporter currents were less than 0.3 μA). Bath flow was controlled by an automated perfusion system (ALA Scientific Instruments, solenoid valves).

For transporter pharmacology, oocytes were clamped at −60 to −90 mV, and continuous current measurements acquired using PowerLab Software and an ADInstruments digitizer. Current signals were lowpass filtered at 20 Hz and acquired at 4-8 Hz. All bath and drug-containing solutions were frog ringers solution containing CaCl₂. Drugs were applied for 10-30 seconds until the induced current reached a new steady-state level, followed by a control solution until baseline currents returned to levels that preceded drug application. The difference current (baseline subtracted from peak current during drug application) reflected the net movement of charge resulting from electrogenic transport and was directly proportional to tranport rate. Recordings were made from a single oocyte for up to 60 min, enabling 30-40 separate compounds to be tested per oocyte. Compound-induced currents were saturable and gave half-maximal values at substrate concentrations comparable to radiolabel competition experiments. To compare results between oocytes expressing different levels of transport activity, a saturating concentration of glycodeoxycholate (300 μM) was used as a common reference to normalize results from test compounds. Using this normalization procedure Vₘₐₓ (i.e. maximal induced current) for different compounds tested on different oocytes could be compared.

TABLE 1
In vitro transport data for selected
compounds on IBAT-expressing cells

| COMPOUND | IC₅₀ (μM) | % Max. (GDC) |
|---|---|---|
| (101) | 83 | 0 |
| (104) | 74 | 25 |
| (102) | 91 | 104 |

IC₅₀ data from radiolabeled competition assay in transporter-expressing CHO cells
% Max response (relative to glycodeoxycholate) at a test compound concentration of 100 μM in transporter-expressing oocytes

TABLE 2
In vitro transport data for selected
compounds on LBAT-expressing cells

| COMPOUND | IC₅₀ (μM) | % Max. (GDC) |
|---|---|---|
| (101) | 5 | ND |
| (104) | 1.8 | ND |
| (102) | 0.2 | ND |

IC₅₀ data from radiolabeled competition assay in transporter-expressing CHO cells
% Max response (relative to glycodeoxycholate) at a test compound concentration of 100 μM in transporter-expressing oocytes
ND = not determined

### EXAMPLE 6

### In Vitro Enzymatic Release of (101) and L-DOPA from (102)

The release of L-DOPA and the intermediate (101) from the prodrug (102) was evaluated in vitro using tissues representative of those involved in the enterohepatic circulation. Similarly, the release of L-DOPA from (101) was examined in the same tissue preparations. Tissues were obtained from commercial sources (e.g., Pel-Freez Biologicals, Rogers, Ark., or GenTest Corporation, Woburn, Mass.). Stability of (102) towards specific enzymes (e.g., carboxypeptidase A, cholylglycine hydrolase) was also evaluated by incubation with the purified enzyme. Experimental conditions used for the in vitro studies are described in the following table. Each preparation was incubated with (102) at 37° C. for one hour. Aliquots (50 μL) were removed at 0, 30, and 60 min and quenched with 0. 1% trifluoroacetic acid in acetonitrile. Samples were then centrifuged and analyzed by LCMS/MS as described in Example 7.

TABLE 3
In Vitro Enzymatic Release of L-DOPA or (101) from (102)

| | | | Percent | Percent |
|---|---|---|---|---|
| | | | of L-Dopa | of (101) |
| | Substrate | | Released | Released |
| Preparation | Concentration | Cofactors | in 60 min | in 60 min* |
| Rat Plasma | 2.0 μM | None | NR | 75 |
| Human Plasma | 2.0 μM | None | NR | 90 |
| Rat Liver S9 | 2.0 μM | NADPH | NR | 35 |
| (0.5 mg/mL) | | | | |
| Human Liver S9 | 2.0 μM | NADPH | NR | 70 |
| (0.5 mg/mL) | | | | |
| Human Intestine S9 | 2.0 μM | NADPH | NR | 95 |
| (0.5 mg/mL) | | | | |
| Cholylglycine | 0.8 μM | None | NR | NR |
| Hydrolase | | | | |
| (87 units/mL) | | | | |

NR—Not released
*XP11215 was further hydrolysed in vitro by cholylglycine hydrolase (95% in 60 min) to release L-Dopa.

### EXAMPLE 7

### Oral Bioavailability of L-DOPA and (101) from the Prodrug (102)

The pharmacokinetics of the prodrug (102) were examined in rats. Three groups of four male Sprague-Dawley rats (200-300 g) with jugular cannulae each received one of the following treatments: A) a single bolus intravenous injection of L-DOPA (75 mg/kg, as a solution in water); B) a single oral dose of L-DOPA (75 mg/kg, as a solution in water) administered by gavage; C) a single oral dose of (102) (267 mg/kg, as a solution in PEG400) administered by gavage. Animals were fasted overnight prior to the study and until 4 hours post-dosing. Serial blood samples were obtained over 48 hours following dosing and blood was processed for plasma by centrifugation. Plasma samples were frozen at −80° C. until analyzed.

Concentrations of L-DOPA in plasma were determined by LC/MS/MS. Plasma (100 μL) was mixed with 10 μL of 500 μ/ml deuterated L-DOPA as internal std, 25 μL of 10% sodium metabisulfite, 300 μL of 2M tris containing 5% EDTA and 30 mg of acid washed aluminum oxide was added to extract L-DOPA. The alumina was washed four times with 300 μL water and extracted with 300 μL of 2.5% formic acid. The extract was analyzed using LC/MS/MS on a 3 μm Phenomenex Luna 4.6×150 mm column. The mobile phases were: A) 0.1% formic acid; B) Acetonitrile with 0.1% formic acid at a flow rate of 0.5 mL/min at 40° C. The gradient was 2% B increasing to 90% B over 3.5 min. The MRM transitions were 198.1/152.0 for L-DOPA and 202.0/155.0 for deuterated L-DOPA. The method was linear over the range 0.02 to 20 μg/mL and the limit of quantitation was 0.02 μg/mL.

Concentrations of (102), and intermediate (101), in plasma samples were determined by LC/MS/MS following precipitation of protein. Plasma (100 μL) was mixed with 300 μL of MeOH and centrifuged at 14,000 rpm for 10 min. The supernatant was analyzed by LC/MS/MS as described above. The MRM transitions were 702.6/152.1 for (102) and 588.5/534.3 for (101).

### EXAMPLE 8

### Synthesis of Cholyl-Amino Acid-L-Dopa (106)

Cholic acid (820 mg, 2 mmol) was dissolved in anhydrous THF (60 mL) and triethylamine (0.70 mL, 5 mmol) added slowly with stirring. The solution was cooled to −5° C. in an ice-salt bath for 30 minutes, and ethyl chloroformate (0.24 mL, 2.4 mmol) added slowly, maintaining the temperature between 0 and 5° C. After addition was complete, the cold mixture was stirred for a total of 90 minutes. A solution containing an amino acid (5 mmol) in water (20 mL) containing saturated NaHCO₃ (25 mL) was added and the mixture stirred for an additional 2 h at room temperature. After removal of the THF in vacuo, saturated NaHCO₃ (15 mL) was added and the aqueous mixture washed with EtOAc (3×10 mL), then the pH adjusted to 3-4 with citric acid. The product was extracted into EtOAc (3×15 mL), and the combined organic phase dried over MgSO_{4,} and concentrated to dryness. The crude products (105) were used directly for coupling to L-Dopa as follows. The compounds were dissolved in anhydrous THF (60 mL) and triethylamine (0.70 mL, 5 mmol) added slowly with stirring. The solutions were cooled to −5° C. in an ice-salt bath for 30 minutes, and ethyl chloroformate (0.24 mL, 2.4 mmol) added slowly, maintaining the temperature between 0 and 5° C. After addition was complete, the cold mixtures were stirred for a total of 90 minutes. A solution containing L-Dopa (5 mmol) in water (20 mL) containing saturated NaHCO₃ (25 mL) was added and the mixtures stirred for an additional 2 h at room temperature. After removal of the THF in vacuo, saturated NaHCO₃ (15 mL) was added and the aqueous mixtures washed with EtOAc (3×10 mL), then the pH adjusted to 3-4 with citric acid. The products were extracted into EtOAc (3×15 mL), and the combined organic phase dried over MgSO₄, and concentrated to dryness. The residues were purified by purified by preparative HPLC, using a Waters Nova-Pak C-18 column (19×300 mm) and eluting with a water/acetonitrile/0.05 % formic acid gradient at 25 mL/min (30% MeCN ramping to 43% in 3 min, then to 53% MeCN by 22 min) to give the pure cholic acid L-Dopa dipeptide derivatives (106). Compounds were characterized by electrospray mass spectrometry as reported below:

Cholyl-Gly-L-Dopa (106a): MS (ESI) m/z 643.7(M−H⁻), 645.7 (M+H⁺).

Cholyl-Val-L-Dopa (106c): MS (ESI) m/z 685.8 (M−H⁻), 687.7 (M+H⁺).

Cholyl-Phe-L-Dopa (106g): MS (ESI) m/z 733.8(M−H⁻), 735.8 (M+H⁺).

Cholyl-Gly-L-Dopa (106a): MS (ESI) m/z 643.7(M−H⁻), 645.7 (M+H⁺).

Cholyl-Norval-L-Dopa (106e): MS (ESI) m/z 685.8 (M−H⁻), 687.7 (M+H⁺).

¹H NMR (CD₃₀D, 400 MHz, characteristic resonances only): 6.64 (d, 1H, J=8 Hz), 6.64 (d, 1H, J=2 Hz), 6.52 (dd, 1H, J=2 Hz, J=8 Hz), 4.60 (m, 1H), 4.32 (m, 1H), 3.06-2.75 (m, 2H), 0.98 (d, 3H, J=6.4 Hz), 0.91 (s, 3H), 0.68 (s, 3H).

Cholyl-Phe-L-Dopa (106g): MS (ESI) m/z 733.8 (M−H⁻), 735.8 (M+H⁺).

¹H NMR (CD₃OD, 400 MHz, characteristic resonances only): 7.22 (m, 5H), 6.64 (m, 1H), 6.52 (m,1H), 4.65-4.55 (m, ₂H), 3.14-3.00 (m, ₂H), 2.89-2.77 (m, 2H), 0.95 (d, 3H, J=6.4 Hz), 0.91 (s, 3H), 0.67 (s, 3H).

Cholyl-Tyr-L-Dopa (106h): MS (ESI) m/z 749.8 (M−H⁻), 751.8 (M+H⁺).

¹H NMR (CD₃OD, 400 MHz, characteristic resonances only): 7.01 (d, 2H, J=8.4 Hz), 6.62 (m, 2H), 6.62 (d, 2H, J=8.4 Hz), 6.51 (m, 1H,), 4.54 (m, ²H), 3.01 (m, 2H), 2.90-2.70 (m, 2H), 0.98 (d, 3H, J=6.4 Hz), 0.91 (s, 3H), 0.68 (s, 3H).

### EXAMPLE 9

### Synthesis of Cholyl-L-Dopa Esters (107)

Cholyl-L-Dopa (101) (120 mg, 0.2 mmol) was dissolved in THF (5 mL) and DIC 25 mg, 0.25 mmol) was added. The solution was treated with a 4-fold molar excess of one of the following alcohols—ethanol, isopropanol, benzyl alcohol, methyl 2,2-dimethyl-3-hydroxypropionate, 1,3-propanediol, ethyl 6-hydroxyhexanoate, 2,2-dimethylaminoethanol at room temperature overnight. The solvent was removed in vacuo and the residues purified by preparative HPLC as described in Example 7 above to afford the pure cholic acid L-Dopa esters (107). Compounds were characterized by electrospray mass spectrometry as reported below:

(107a): MS (ESI) m/z 614.4 (M−H⁻), 616.3 (M+H⁺).

(107b): MS (ESI) m/z 628.5 (M−H⁻), 630.4 (M+H⁺).

(107c): MS (ESI) m/z 676.4 (M−H⁻), 678.3 (M+H⁺).

(107d): MS (ESI) m/z 700.4 (M−H⁻), 702.4 (M+H⁺).

(107e): MS (ESI) m/z 644.4 (M−H⁻), 646.3 (M+H⁺).

(107f): MS (ESI) m/z 728.4 (M−H⁻), 730.3 (M+H⁺).

(107g): MS (ESI) m/z 657.5 (M−H⁻), 659.4 (M+H⁺).

The procedures set forth above for L-DOPA conjugated to a bile acid are also applicable to a catechol O-methyl transferase inhibitor, an inhibitor of a L-aromatic amino acid decarboxylase, and derivatives of L-DOPA. That is to say that by following the procedures set forth above and using the appropriate starting materials, a catechol O-methyl transferase inhibitor, an inhibitor of a L-aromatic amino acid decarboxylase, or a derivatives of L-DOPA can be conjugated to such bile acids. It is understood, of course, that the use of appropriate protecting groups and reaction conditions to add and remove such groups may be necessary but such is well within the skill of the art.

In addition, the above procedures as well as the attached figures and supporting description thereof evidence that any drug containing a carboxyl group, an amine group and/or a hydroxyl group can be attached to bile acids to effect compounds having prolonged release in vivo.

Examples of drugs containing carboxyl groups include, for instance, angiotensin-converting enzyme inhibitors such as alecapril, captopril, 1-[4-carboxy-2-methyl-2R, 4R-pentanoyl]-2,3-dihydro-2S-indole-2-carboxylic acid, enalaprilic acid, lisinopril, N-cyclopentyl-N-[3-[(2,2-dimethyl-1-oxopropyl)thio]-2-methyl-1-oxopropyl]glycine, pivopril, quinaprilat, (2R, 4R)-2-hydroxyphenyl)-3-(3-mercaptopropionyl)-4-thiazolidinecarboxylic acid, (S) benzamido-4-oxo-6-phenylhexenoyl-2-carboxypyrrolidine, [2S-1 [R^{*}(R^{*}))]] 2α, 3β, 7αβ]-1 [2-[[1carboxy-3-phenylpropyl]-amino]-1-oxopropyl]octahydro-1H-indole-2-carboxylic acid, [3S-1[R^{*}(R^{*}))]], 3R^{*}]-2-[2-[[1-carboxy-3-phenylpropyl]-amino]-1-oxopropyl]-1,2,3,4-tetrahydro-3-isoquinolone carboxylic acid and tiopronin; cephalosporin antibiotics such as cefaclor, cefadroxil, cefamandole, cefatrizine, cefazedone, cefazuflur, cefazolin, cefbuperazone, cefixime, cefmenoxime, cefinetazole, cefodizime, cefonicid, cefoperazone, ceforanide, cefotaxime, cefotefan, cefotiam, cefoxitin, cefpimizole, cefpirome, cefpodoxime, cefroxadine, cefsulodin, cefpiramide, ceftazidime, ceftezole, ceftizoxime, ceftriaxone, cefuroximne, cephacetrile, cephalexin, cephaloglycin, cephaloridine, cephalosporin, cephanone, cephradine and latamoxef; penicillins such as amoxycillin, ampicillin, apalcillin, azidocillin, azlocillin, benzylpencillin, carbenicillin, carfecillin, carindacillin, cloxacillin, cyclacillin, dicloxacillin, epicillin, flucloxacillin, hetacillin, methicillin, mezlocillin, nafcillin, oxacillin, phenethicillin, piperazillin, sulbenicillin, temocillin and ticarcillin; thrombin inhibitors such as argatroban, melagatran and napsagatran; influenza neuraminidase inhibitors such as zanamivir and BCX-1812; non-steroidal antiinflammatory agents such as acametacin, alclofenac, alminoprofen, aspirin (acetylsalicylic acid), 4-biphenylacetic acid, bucloxic acid, carprofen, cinchofen, cinmetacin, clometacin, clonixin, diclenofac, diflunisal, etodolac, fenbufen, fenclofenac, fenclosic acid, fenoprofen, ferobufen, flufenamic acid, flufenisal, flurbiprofin, fluprofen, flutiazin, ibufenac, ibuprofen, indomethacin, indoprofen, ketoprofen, ketorolac, lonazolac, loxoprofen, meclofenamic acid, mefenamic acid, 2-(8-methyl-10,11-dihydro-11-oxodibenz[b,f]oxepin-2-yl)propionic acid, naproxen, nifluminic acid, O-(carbamoylphenoxy)acetic acid, oxoprozin, pirprofen, prodolic acid, salicylic acid, salicylsalicylic acid, sulindac, suprofen, tiaprofenic acid, tolfenamic acid, tohnetin and zopemirac; prostaglandins such as ciprostene, 16-deoxy-16-hydroxy-16-vinyl prostaglandin E₂, 6,16-dimnethylprostaglandin E₂ , epoprostostenol, meteneprost, nileprost, prostacyclin, prostaglandins E₁, E₂, or F₂_{_{α}}, and thromboxane A₂; quinolone antibiotics such as acrosoxacin, cinoxacin, ciprofloxacin, enoxacin, flumequine, naladixic acid, norfloxacin, ofloxacin, oxolinic acid, pefloxacin, pipemidic acid and piromidic acid; other antibiotics such as aztreonam, imipenem, meropenem and related carbopenem antibiotics.

Representative drugs containing amine groups include: acebutalol, albuterol, alprenolol, atenolol, bunolol, bupropion, butopamine, butoxamine, carbuterol, cartelolol, colterol, deterenol, dexpropanolol, diacetolol, dobutamine, exaprolol, exprenolol, fenoterol, fenyripol, labotolol, levobunolol, metolol, metaproterenol, metoprolol, nadolol, pamatolol, penbutalol, pindolol, pirbuterol, practolol, prenalterol, primidolol, prizidilol, procaterol, propanolol, quinterenol, rimiterol, ritodrine, solotol, soterenol, sulfiniolol, sulfinterol, sulictidil, tazaolol, terbutaline, timolol, tiprenolol, tipridil, tolamolol, thiabendazole, albendazole, albutoin, alendronate, alinidine, alizapride, amiloride, aminorex, aprinocid, cambendazole, cimetidine, cisapride, clonidine, cyclobenzadole, delavirdine, efegatrin, etintidine, fenbendazole, fenmetazole, flubendazole, fludorex, icadronate, lobendazole, mebendazole, metazoline, metoclopramide, methylphenidate, mexiletine, neridronate, nocodazole, oxfendazole, oxibendazole, oxmetidine, pamidronate, parbendazole, pramipexole, prazosin, procainamide, ranitidine, tetrahydrazoline, tiamenidine, tinazoline, tiotidine, tocainide, tolazoline, tramazoline, xylometazoline, dimethoxyphenethylamine, N-[3(R)-[ 2-piperidin-4-yl)ethyl]-2-piperidone-1-yl]acetyl-3(R)-methyl-β-alanine, adrenolone, aletamine, amidephrine, amphetamine, aspartame, bamethan, betahistine, clorprenaline, chlortermine, dopamine, ephrinephrine etryptamine, fenfluramine, methyldopamine, norepinephrine, tocainide, enviroxime, nifedipine, nimodipine, triamterene, norfloxacin and similar compounds such as pipedemic acid, 1-ethyl-6-fluoro- 1,4dihydro-4-oxo-7-(1-piperazinyl)-1, 8-napthyridine-3-carboxylic acid, 1-cyclopropyl-6-fluoro-1,4-dihydro-4-oxo-7-(piperazinyl)-3-quinolinecarboxylic acid.

Representative drugs containing hydroxy groups include: steroidal hormones such as allylestrenol, cingestol, dehydroepiandrosteron, dienostrol, diethylstilbestrol, dimethisteron, ethyneron, ethynodiol, estradiol, estron, ethinyl estradiol, ethisteron, lynestrenol, mestranol, methyl testosterone, norethindron, norgestrel, norvinsteron, oxogeston, quinestrol, testosteron and tigestol; tranquilizers such as dofexazepam, hydroxyzin, lorazepam and oxazepam; neuroleptics such as acetophenazine, carphenazine, fluphenazine, perphenyzine and piperaetazine; cytostatics such as aclarubicin, cytarabine, decitabine, daunorubicin, dihydro-5-azacytidine, doxorubicin, epirubicin, estramustin, etoposide, fludarabine, gemcitabine, 7-hydroxychlorpromazin, nelarabine, neplanocin A, pentostatin, podophyllotoxin, tezacitabine, troxacitabine, vinblastin, vincristin, vindesin; hormones and hormone antagonists such as buserilin, gonadoliberin, icatibrant and leuprorelin acetate; antihistamines such as terphenadine; analgesics such as diflunisal, naproxol, paracetamol, salicylamide and salicyclic acid; antibiotics such as azidamphenicol, azithromycin, camptothecin, cefamandol, chloramphenicol, clarithromycin, clavulanic acid, clindamycin, demeclocyclin, doxycyclin, erythromycin, gentamycin, imipenem, latamoxef, metronidazole, neomycin, novobiocin, oleandomycin, oxytetracyclin, tetracycline, thiamenicol and tobramycin; antivirals such as acyclovir, d4C, ddC, DMDC, Fd4C, FddC, FMAU, FTC, 2′-fluoro-ara-dideoxyinosine, ganciclovir, lamivudine, penciclovir, SddC, stavudine, 5-trifluoromethyl-2′-deoxyuridine, zalcitabine and zidovudine; bisphosphonates such as EB-1053, etidronate, ibandronate, olpadronate, residronate, YH-529 and zolendronate; protease inhibitors such as ciprokiren, enalkiren, ritonavir, saquinavir and terlakiren; prostaglandins such as arbaprostil, carboprost, misoprostil and prostacydin; antidepressives such as 8-hydroxychlorimipramine and 2-hydroxyimipramine; antihypertonics such as sotarol and fenoldopam; anticholinerogenics such as biperidine, procyclidin and trihexyphenidal; antiallergenics such as cromolyn; glucocorticoids such as betamethasone, budenosid, chlorprednison, clobetasol, clobetasone, corticosteron, cortisone, cortodexon, dexamethason, flucortolon, fludrocortisone, flumethasone,flunisolid, fluprednisolon, flurandrenolide, flurandrenolon acetonide, hydrocortisone, meprednisone, methylpresnisolon, paramethasone, prednisolon, prednisol, triamcinolon and triamcinolon acetonide; narcotic agonists and antagonists such as apomorphine, buprenorphine, butorphanol, codein, cyclazocin, hydromorphon, ketobemidon, levallorphan, levorphanol, metazocin, morphine, nalbuphin, nalmefen, naloxon, nalorphine, naltrexon, oxycodon, oxymorphon and pentazocin; stimulants such asmazindol and pseudoephidrine; anaesthetics such as hydroxydion and propofol; β-receptor blockers such as acebutolol, albuterol, alprenolol, atenolol, betazolol, bucindolol, cartelolol, celiprolol, cetamolol, labetalol, levobunelol, metoprolol, metipranolol, nadolol, oxyprenolol, pindolol, propanolol and timolol; α-sympathomimetics such as adrenalin, metaraminol, midodrin, norfenefrin, octapamine, oxedrin, oxilofrin, oximetazolin and phenylefrin; β-sympathomimetics such as bamethan, clenbuterol, fenoterol, hexoprenalin, isoprenalin, isoxsuprin, orciprenalin, reproterol, salbutamol and terbutalin; bronchodilators such as carbuterol, dyphillin, etophyllin, fenoterol, pirbuterol, rimiterol and terbutalin; cardiotonics such as digitoxin, dobutamin, etilefrin and prenalterol; antimycotics such as amphotericin B, chlorphenesin, nystatin and perimycin; anticoagulants such as acenocoumarol, dicoumarol, phenprocoumon and warfarin; vasodilators such as bamethan, dipyrimadol, diprophyllin, isoxsuprin, vincamin and xantinol nicotinate; antihypocholesteremics such as compactin, eptastatin, mevinolin and simvastatin; miscellaneous drugs such as bromperidol (antipsychotic), dithranol (psoriasis) ergotamine (migraine) ivermectin (antihelminthic), metronidazole and secnizadole (antiprotozoals), nandrolon (anabolic), propafenon and quinadine (antiarythmics), quetiapine (CNS), serotonin (neurotransmitter) and silybin (hepatic disturbance).

From the foregoing description, various modifications and changes in the above described methods will occur to those skilled in the art. All such modifications coming within the scope of the appended claims are intended to be included therein.

What is claimed is:

1. A compound of formula (I): 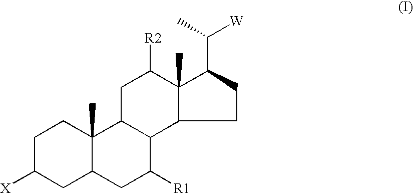
wherein:
R¹ is selected from the group consisting of hydrogen and OH;
R² is selected from the group consisting of hydrogen and OH;
X is selected from the group consisting of OH and D-Y-, where Y is selected from the group consisting of a covalent bond and a cleavable linker group covalently connecting D to the steroid;
D is a member selected from the group consisting of L-DOPA, a catechol O-methyl transferase inhibitor, an inhibitor of a L-aromatic amino acid decarboxylase, and derivatives of L-DOPA;
W is selected from the group consisting of (a) a substituted alkyl group containing a moiety which is negatively charged at physiological pH, which moiety is selected from the group consisting of —COOH, —SO₃H, —SO₂H, —P(O)(OR⁶)(OH), —OP(O)(OR⁶)(OH), —OSO₃H and pharmaceutically acceptable salts thereof, where R⁶ is selected from the group consisting of alkyl, substituted alkyl, aryl and substituted aryl; and (b) a group of the formula:
-M-Y′-D′
wherein:
M is selected from the group consisting of —CH₂OC(O)— and —CH₂CH₂C(O)—;
Y′ is a covalent bond or a cleavable linker group covalently connecting D′ to M;
D′ is a member selected from the group consisting of L-DOPA, a catechol O-methyl transferase inhibitor, an inhibitor of a L-aromatic amino acid decarboxylase, and derivatives of L-DOPA;
with the proviso that either X is -Y-D and/or W is -M-Y′-D′
wherein the compound of formula (I) above is a substrate for an intestinal bile acid transporter;
or a pharmaceutically acceptable salt thereof.

2. A compound of formula (I-a): 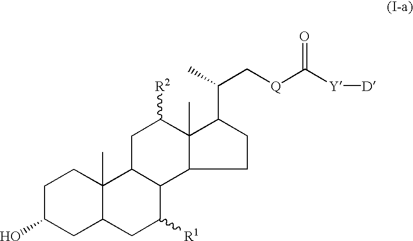
wherein:
Y′ is selected from the group consisting of a covalent bond and a cleavable linker group covalently connecting D′ to the C-24 position of the steroid;
D′ is a member selected from the group consisting of L-DOPA, a catechol O-methyl transferase inhibitor, an inhibitor of a L-aromatic amino acid decarboxylase, and derivatives of L-DOPA;
Q is CH₂ or O;
R¹ is selected from the group consisting of H and OH;
R² is selected from the group consisting of H and OH;
wherein the compound of formula (I-a) above is a substrate for an intestinal bile acid transporter;
or pharmaceutically acceptable salts thereof.

3. A compound of the formula (I-b): 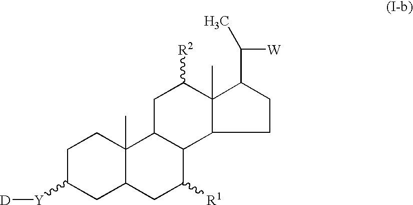
wherein:
Y is selected from the group consisting of a covalent bond and a cleavable linker group covalently connecting D to the steroid;
D is a member selected from the group consisting of L-DOPA, a catechol O-methyl transferase inhibitor, an inhibitor of a L-aromatic amino acid decarboxylase, and derivatives of L-DOPA;
R¹ is selected from the group consisting of H and OH;
R² is selected from the group consisting of H and OH;
W is a substituted alkyl group containing a moiety which is negatively charged at physiological pH, which moiety is selected from the group consisting of —COOH, —SO₃H, —SO₂H, —P(O)(OR⁶)(OH), —OP(O)(OR⁶)(OH), —OSO₃H, and pharmaceutically acceptable salts thereof, where R⁶ is selected from the group consisting of alkyl, substituted alkyl, aryl and substituted aryl;
wherein the compound of formula (I-b) above is a substrate for an intestinal bile acid transporter;
or pharmaceutically acceptable salts thereof.

4. A compound of formula (I-c): 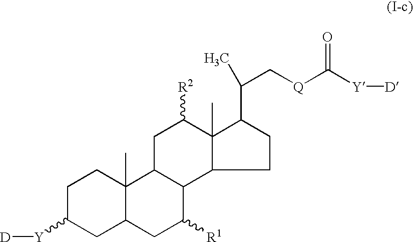
wherein:
Y′ is selected from the group consisting of a covalent bond and a cleavable linker group covalently connecting D′ to the C-24 position of the steroid;
D′ is a member selected from the group consisting of L-DOPA, a catechol O-methyl transferase inhibitor, an inhibitor of a L-aromatic amino acid decarboxylase, and derivatives of L-DOPA;
Y is selected from the group consisting of a covalent bond and a cleavable linker group covalently connecting D to the steroid;
D is a member selected from the group consisting of L-DOPA, a catechol O-methyl transferase inhibitor, an inhibitor of a L-aromatic amino acid decarboxylase, and derivatives of L-DOPA;
Q is CH₂ or O;
R¹ is selected from the group consisting of H and OH;
R² is selected from the group consisting of H and OH;
wherein the compound of formula (I-c) above is a substrate for an intestinal bile acid transporter;
or pharmaceutically acceptable salts thereof.

5. The compound according to claim 1, wherein W is selected from the group consisting of —CH₂CH₂CO₂H, —CH₂CH₂CONHCH₂CO₂H, —CH₂CH₂CONHCH₂CH₂SO₃H, and pharmaceutically acceptable salts thereof.

6. The compound according to claim 1, wherein W is selected from the group of the formula:
-M-Y′-D′
wherein:
M is selected from the group consisting of —CH₂₀C(O)— and —CH₂CH₂C(O)—;
Y′ is a covalent bond or a cleavable linker group covalently connecting D′ to M;
D′ is a member selected from the group consisting of L-DOPA, a catechol O-methyl transferase inhibitor, an inhibitor of a L-aromatic amino acid decarboxylase, and derivatives of L-DOPA;
or a pharmaceutically acceptable salt thereof.

7. The compound according to claim 1, wherein R¹ and R² are selected from the group consisting of the following combinations:
R¹ and R² are α-OH;
R¹ is α-OH and R² is H;
R¹ is β-OH and R² is H;
R¹ is H and R² is α-OH;
R¹ is β-OH and R² is α-OH; and
R¹ and R² are H,
or a pharmaceutically acceptable salt thereof.

8. The compound according to claim 7, wherein W is selected from the group consisting of —CH₂CH₂CO₂H, —CH₂CH₂CONHCH₂CO₂H, —CH₂CH₂CONHCH₂CH₂SO₃H, and pharmaceutically acceptable salts thereof.

9. The compound according to claim 7, wherein W is selected from the group of the formula:
-M-Y′-D′
wherein:
M is selected from the group consisting of —CH₂OC(O)— and —CH₂CH₂C(O)—;
Y′ is a covalent bond or a cleavable linker group covalently connecting D′ to M;
D′ is a member selected from the group consisting of L-DOPA, a catechol O-methyl transferase inhibitor, an inhibitor of a L-aromatic amino acid decarboxylase, and derivatives of L-DOPA;
or a pharmaceutically acceptable salt thereof.

10. The compound according to claim 1 wherein D and/or D′ is L-DOPA or a derivative of L-DOPA, or a pharmaceutically acceptable salt thereof.

11. The compound according to claim 1, wherein X is -Y-D and D is L-DOPA, a derivative of L-DOPA, or a pharmaceutically acceptable salt thereof.

12. The compound according to claim 11, wherein W is -M-Y′-D′, or a pharmaceutically acceptable salt thereof.

13. The compound according to claim 12, wherein D′ is L-DOPA, a derivative of L-DOPA, or a pharmaceutically acceptable salt thereof.

14. The compound according to claim 12, wherein D′ is a catechol O-methyl transferase inhibitor or a pharmaceutically acceptable salt thereof.

15. The compound according to claim 12, wherein D′ is a L-aromatic amino acid decarboxylase inhibitor or a pharmaceutically acceptable salt thereof.

16. The compound according to claim 1, wherein X is -Y-D, or a pharmaceutically acceptable salt thereof.

17. The compound according to claim 16, wherein D is a catechol O-methyl transferase inhibitor or a pharmaceutically acceptable salt thereof.

18. The compound according to claim 16, wherein D is a L-aromatic amino acid decarboxylase inhibitor or a pharmaceutically acceptable salt thereof.

19. The compound according to Claim 15 or 18, wherein the inhibitor of L-aromatic amino acid decarboxylase is carbidopa or benserazide.

20. The compound according to claim 14 or 17, wherein the catechol O-methyl transferase inhibitor is entacapone, nitecapone or tolcapone.

21. The compound according to claim 1, wherein Y and Y′ are represented by the formula -X^{*}-Y^{*}-Z- where X^{*} is the linker chemistry for attachment to the drug D or D′; Y^{*} is a covalent bond or a linker moiety; and Z is the linker chemistry for attachment to the steroid;
Wherein:
X^{*} is selected from the group consisting of —OC(O)—, —OC(O)NR⁷—, —OC(O)OCR¹¹R¹²O, —OC(O)OCR¹¹R ¹²OC(O)—, —OC(O)OCR¹¹R¹²OC(O)O—, —OC(O)OCR¹¹R¹²OC(O)NR⁷—, —NR⁷C(O)O—, —NR⁷C(O)—, —NR⁷C(O)OCR¹¹R¹²OC(O)—, —NR⁷C(O)OCR¹¹R¹²OC(O)O—, —NR⁷CH₂NR⁷C(O)—, —C(O)O—, —C(O)S—, —C(O)NR⁷—, —C(O)NR⁷C(O)R⁷—, —C(O)CR¹¹R ¹²O—, —C(O)OCR¹¹R¹²OC(O)—, —C(O)OCR¹¹R¹²OC(O)O—, —C(O)OCH₂C(O)NR⁷—, —C(O)OCH₂CH₂NR⁷C(O)—, —C(O)OCH₂NR⁷C(O)—, —C(O)OCR¹¹R¹²OC(O)NR⁷—, with the underlined atom being derived from a hydroxyl, NH, carboxylic acid moiety of the drug D or D′;
each R⁷ is independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl;
R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring;
Z is selected from the group consisting of a bond, —O—, —S—, —C(O)O—, —OC(O)O—, —NR⁷C(O)O—, —OC(O)NR⁷—, —OP(O)(OR⁶)O—, —P(O)(OR⁶)O—, —NR⁷P(O)(OR⁶)O—, —C(O)NR⁷—, —NR⁷C(O)NR⁷—, —NR⁷C(O)NR⁷—, —S(O)₂NR⁷—, —S(O)—, —S(O)₂—, —C(O)S—, —ON═, —C(O)ON═, —NR⁷C(O)ON═, —C(O)OCR¹¹R¹²ON═, and a C═C linkage, wherein R⁶, R⁷, R¹¹, and R¹² are defined as above;
Y^{*} is a bond or a bivalent hydrocarbyl radical of 1 to 18 atoms having at least one alkylene, alkenylene or alkynylene group, with said at least one alkylene, alkenylene or alkynylene group optionally replaced with —O—, —S—, —NR⁷—, —C(O)—, —C(S)—, —OC(O)—, —C(O)O—, —SC(O)—, —C(O)S—, —SC(S)—, —C(S)S—, —C(O)NR⁷—, —NR⁷C(O)—, arylene, substituted arylene, cycloalkylene, substituted cycloalkylene, cycloalkenylene, substituted cycloalkenylene, bivalent heterocyclic group or substituted bivalent heterocyclic group.

22. The compound according to claim 21, wherein said bivalent hydrocarbyl radical, Y^{*} , is 1 to 10 atoms in length.

23. The compound according to claim 22, wherein said bivalent hydrocarbyl radical, Y^{*}, is 1 to 6 atoms in length.

24. The compound according to claim 21, wherein -X^{*}-Y^{*}-Z- is selected from the group consisting of a carbonyl group, thiocarbonyl group and radicals of formulae (vi) to (xlviii): 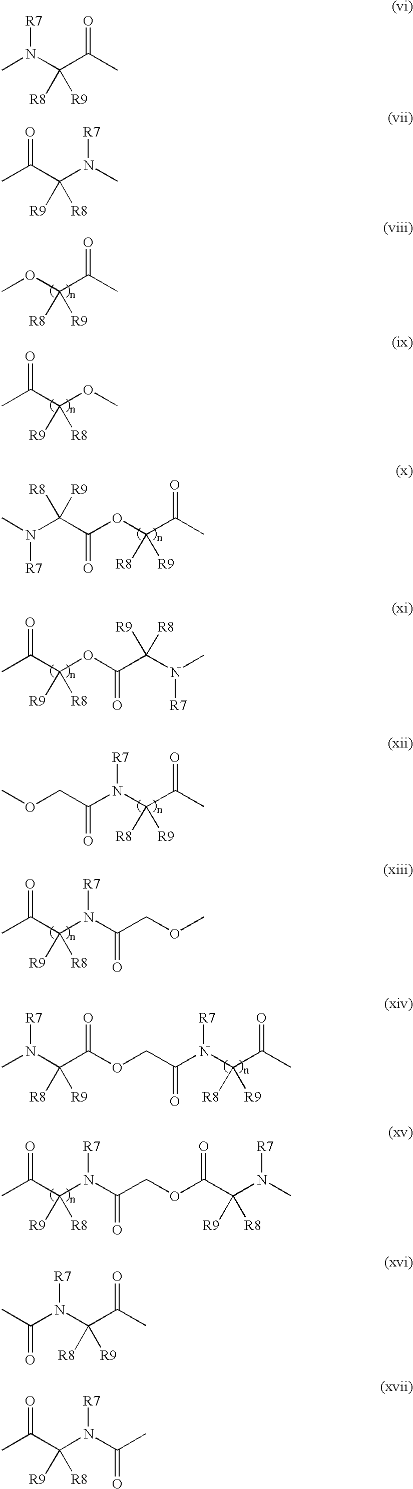
wherein:
n is an integer of 1 to 6;
each R⁷, R⁸ and R⁹ are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or, when R⁷ and R⁹ are present and attached to adjacent atoms, then R⁷ and R⁹ together with the atoms to which they are attached form a cycloalkyl, substituted cycloalkyl, heterocycle or substituted heterocyclic ring;
R¹¹ and R ¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring;
or pharmaceutically acceptable salts thereof.

25. The compound according to claim 2 having formulae (I-a-1) or (I-a-2): 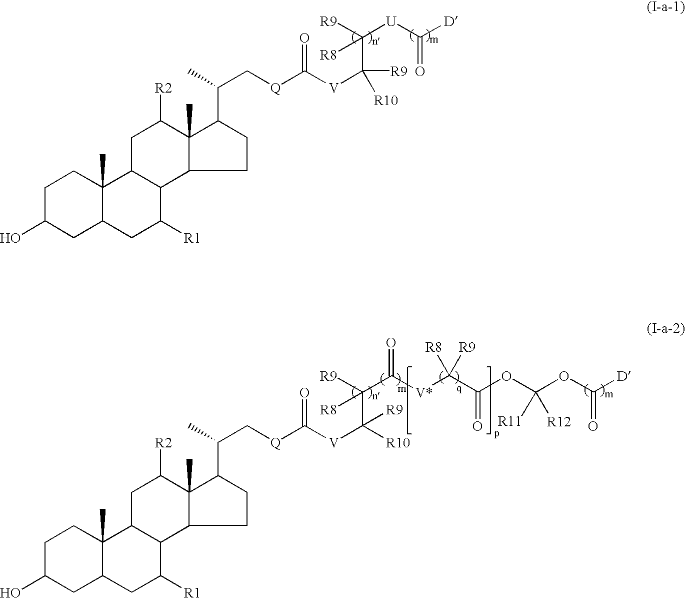
wherein:
D′ is a member selected from the group consisting of L-DOPA, a catechol O-methyl transferase inhibitor, an inhibitor of a L-aromatic amino acid decarboxylase, and derivatives of L-DOPA;
Q is CH₂ or O;
R¹ and R² are one of the following combinations:
R¹ and R² are α-OH;
R¹ is α-OH and R² is H;
R¹ is β-OH and R² is H;
R¹ is H and R² is α-OH;
R¹ is β-OH and R² is α-OH; or
R¹ and R² are H;
V and V^{*} are independently NR⁷, O, S or CR⁸R⁹;
U is NR⁷, O, S;
R¹⁰ is R⁸ or (CR⁸R⁹)ᵣT;
T is selected from the group consisting of CO₂H, SO₃H, OSO₃H, SO₂H, P(O)(OR⁶)(OH), OP(O)(OR⁶)(OH) and pharmaceutically acceptable salts thereof;
each m is 0 or 1;
n′ is 0, 1, 2, 3 or 4;
p is 0, 1, 2;
each q is independently 1, 2, 3 or 4;
r is 0 or 1;
R⁶ is selected from the group consisting of alkyl, substituted alkyl, aryl and substituted aryl;
R⁷, R⁸ and R⁹ are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R⁸ and R⁹ together with the atoms to which they are attached form a cycloalkyl, substituted cycloalkyl, heterocycle or substituted heterocyclic ring, or, when R⁷ and R⁹ are present and attached to adjacent atoms, then R⁷ and R⁹ together with the atoms to which they are attached form a cycloalkyl, substituted cycloalkyl, heterocycle or substituted heterocyclic ring;
R¹¹ and R¹² are independently hydrogen, alkyl, substituted alkyl, alkenyl, substituted alkenyl, alkynyl, substituted alkynyl, cycloalkyl, substituted cycloalkyl, heterocycle, substituted heterocycle, aryl, substituted aryl, heteroaryl, substituted heteroaryl or R¹¹ and R¹² together with the atoms to which they are attached form a cycloalkyl, substituted cycloalkyl, heterocycle or substituted heterocyclic ring;
or pharmaceutically acceptable salts thereof.

26. A compound of claim 2 having formula (I-a) wherein;
Q is CH_{2;}
R¹¹ and R¹² are α-OH;
Y′ is derived from an α-amino acid; and
D′ is a derivative of L-DOPA.
or a pharmaceutically acceptable salt thereof.

27. The compound of claim 26, wherein Y′ is derived from one of the 20 genetically encoded amino acids.

28. The compound of claim 27 having formula (xlix): 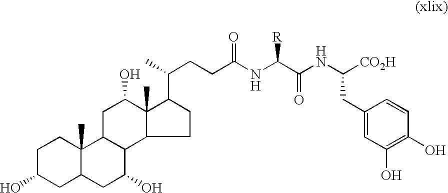
wherein:
R is selected from the group consisting of hydrogen, CHMe₂, CH₂Ph, and CH₂(p-C₆H₄OH);
or a pharmaceutically acceptable salt thereof.

29. A compound of claim 2 having formula (1): 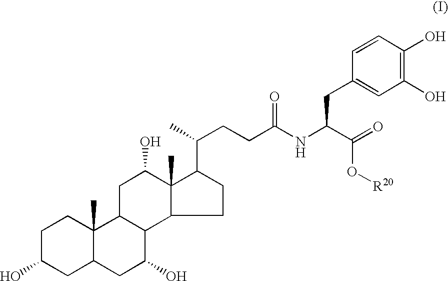
wherein:
R²⁰ is selected from the group consisting of alkyl, substituted alkyl, alkenyl, substituted alkenyl, alkynyl, substituted alkynyl, cycloalkyl, substituted cycloalkyl, aryl, substituted aryl, aralkyl, substituted aralkyl, heteroaryl and substituted heteroaryl;
or a pharmaceutically acceptable salt thereof.

30. The compound of claim 29 wherein R²⁰ is benzyl or substituted benzyl; or a pharmaceutically acceptable salt thereof.

31. A compound of claim 2 having formula (li): 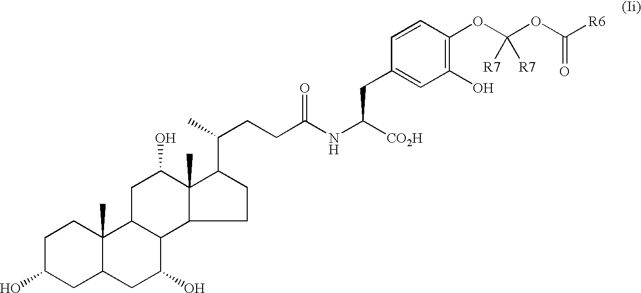
wherein:
R⁶ is selected from the group consisting of alkyl, substituted alkyl, cycloalkyl, substituted cycloalkyl, aryl, substituted aryl, aralkyl, substituted aralkyl, heteroaryl and substituted heteroaryl; and
each R⁷ is independently selected from the group consisting of hydrogen, alkyl, substituted alkyl, cycloalkyl, substituted cycloalkyl, aryl, substituted aryl, aralkyl, substituted aralkyl, heteroaryl and substituted heteroaryl;
or a pharmaceutically acceptable salt thereof.

32. The compound of claim 31 wherein R⁶ is selected from the group consisting of lower alkyl, phenyl, substituted phenyl, benzyl, substituted benzyl and R⁷ is selected from the group consisting of hydrogen and lower alkyl.

33. The compound of claim 32 wherein R⁶ is selected from the group consisting of methyl and tert-butyl and R⁷ is selected from the group consisting of hydrogen and methyl.

34. The compound of claim 25, wherein the L-aromatic amino acid decarboxylase inhibitor is carbidopa or benserazide and the catechol O-methyl transferase inhibitor is entacapone, nitecapone or tolcapone.

35. A pharmaceutical composition comprising a pharmaceutically acceptable excipient and an effective amount of a compound according to any of claims 1 through 4.

36. A method for treating Parkinson's in a subject in need of the treatment, comprising administering a pharmaceutical composition according to claim 35.

37. A compound of formula
D-Y-T
wherein:
D is a member selected from the group consisting of L-DOPA, a catechol O-methyl transferase inhibitor, an inhibitor of a L-aromatic amino acid decarboxylase, and derivatives of L-DOPA;
Y is a cleavable linker; and
T is a substrate for an intestinal bile acid transporter.
